(19) United States
(12) Patent Application Publication
(10) Pub. No.: US 2012/0093917 A1
(43) Pub. Date: Apr. 19, 2012

(54) METNASE AND INTNASE INHIBITORS AND THEIR USE IN TREATING CANCER

(76) Inventors: Robert Hromas, Gainsville, FL (US); Andrei Leitao, Albuquerque, NM (US); Tudor I. Oprea, Albuquerque, NM (US); Larry A. Sklar, Albuquerque, NM (US); Elizabeth A. Williamson, Gainsville, FL (US); Justin Wray, Albuquerque, NM (US)

(21) Appl. No.: 13/255,708

(22) PCT Filed: Mar. 31, 2010
(86) PCT No.: PCT/US2010/029462

§ 371 Date: Oct. 31, 2011

Related U.S. Application Data
(60) Provisional application No. 61/211,723, filed on Apr. 2, 2009.
(60) Provisional application No. 61/274,867, filed on Aug. 21, 2009.
(60) Provisional application No. 61/274,852, filed on Aug. 21, 2009.

(51) Int. Cl. A61K 31/47; A61K 31/192; A61K 31/4375; A61K 31/437; A61K 31/4412; A61K 31/513; A61K 31/496; A61K 31/7048; A61P 35/00; A61P 35/02; A61P 35/04; A61K 31/704; A61K 38/20; A61K 39/395; A61K 33/36; A61K 38/50; A61K 33/24; A61K 31/7068; A61K 9/127; A61N 5/00; A61K 31/382

(52) U.S. Cl. 424/450; 514/312; 514/432; 514/569; 514/300; 514/301; 514/348; 514/269; 514/253.03; 514/27; 514/34; 424/85.2; 424/133.1; 424/623; 424/94.6; 424/649; 514/49; 600/1

(57) ABSTRACT

This invention relates to novel cancer treatment compositions and associated therapeutic methods. More particularly, this invention relates in part to small chemical inhibitors of DNA repair proteins (Metnase) and to a therapeutic method that utilizes the inhibitors to increase the effectiveness of cancer treatment protocols.

### RELATED APPLICATIONS

This application claims the benefit of priority of U.S. provisional applications U.S.61/274,852, filed Aug. 21, 2009 entitled “Metnase Inhibitors and Their Use in Treating Cancer”, U.S.61/274,867, filed Aug. 21, 2009, entitled “Intnase/Gypsy Integrase-1 Inhibitors and Their Use in Treating Cancer” and U.S.61/211,723, filed Apr. 2, 2009, entitled “Targeting Transposase Domain Proteins Defines a New Class of Cancer Chemotherpeutic Agents”, each of which applications is incorporated by reference in its entirety herein.

### FIELD OF THE INVENTION

This invention relates to novel cancer treatment compositions and associated therapeutic methods. More particularly, this invention relates in part to small chemical inhibitors of DNA replication/repair proteins Metnase (also called SETMAR) and/or the related Intnase (also termed Gypsy Integrase-1, Gypsy Retransposon Integrase 1, or GIN-1) and to a therapeutic method that utilizes the inhibitors to increase the effectiveness of cancer treatment protocols.

### BACKGROUND OF THE INVENTION

Most cancer chemotherapy and radiation therapy kills cancer cells by damaging their DNA. Cancer cells resist therapy and relapse by increasing their ability to repair their DNA. Identifying the DNA repair proteins that cancer cells use to repair their DNA after therapy would provide new targets to enhance therapy and prevent relapse. Small chemical inhibitors of those target DNA proteins could prevent cancer cells from escaping therapy.

### OBJECTS OF THE INVENTION

It is an object of the present invention to provide a novel composition or group of related compositions that are useful in the treatment of cancer.

It is a more particular object of the present invention to provide a novel composition or group of related compositions that are useful in inhibiting the DNA repair proteins that aid cancer cells in resisting therapy and relapse.

It is yet another object of the present invention to provide novel pharmaceutical compositions which combine a Metnase or Intnase inhibitor with a traditional anticancer agent.

It is a further object of the invention to provide combination therapies which utilize a Metnase or Intnase inhibitor as described herein in combination with a traditional anticancer agent or other therapy, especially including radiation therapy in the treatment of cancer.

Another object of the present invention is to provide associated cancer treatment protocols and therapies.

Any one or more of these and/or other objects of the present invention will be apparent from the drawings and descriptions herein.

### BRIEF DESCRIPTION OF THE FIGURES

FIG. 1A is a diagram of the protein domains of Metnase (also termed SETMAR).

FIG. 1B is a graph illustrating the ability of the Metnase protein to increase Non-Homologous End-joining repair of DNA double-strand breaks when over-expressed (pCDNA-Metnase) and decrease it when repressed (siRNA Metnase).

FIG. 1C is a diagram comparing the Integrase domains of Intnase (also termed Gypsy Retransposon Integrase 1, GIN-1, or Gypsy Integrase-1), Rous Sarcoma Virus (here RSV), and Human Immunodeficiency Virus (here HIV).

FIG. 1D is a chart illustrating the structure properties of the Integrase family members in FIG. 1C.

FIG. 2A is a graph depicting percentage survival of breast cancer cells as a function of VP-16 (also called etoposide) application, with (shMetnase) and without repression (shGFP) of Metnase expression.

FIG. 2B is a graph depicting percentage survival of breast cancer cells as a function of Adriamycin application, with (shMetnase) and without repression (shGFP) of Metnase expression.

FIG. 2C is a bar graph of the percentage of apoptotic (annexin-V⁺ or PI expression) breast cancer cells after application of adriamycin with (shMetnase) and without (shGFP) repression of Metnase.

FIG. 2D shows Intnase/Gypsy Integrase-1 protein domain analysis and sequence.

FIG. 3A is a graph showing growth of leukemia cells as a function of time with (Metnase KD) and without (Vector Control) repression of Metnase.

FIG. 3B is a bar graph of the percentage of apoptotic leukemia cells (annexin-V⁺) after application of VP-16 with (shMetnase) and without (shGFP) repression of Metnase

FIG. 3C is a graph showing growth of leukemia cells as a function of time after treatment with 0.5 μM of VP-16 with (triangles) or without (circles) prior repression of Metnase expression.

FIG. 3D shows human tissue expression of Intnase/Gypsy Integrase-1 using RT-PRC. The figure shows that this gene expressed in almost all human tissues tested.

FIG. 4A is a graph showing growth of leukemia cells as a function of time after treatment with 1.0 μM of VP-16 with (triangles) or without (circles) prior repression of Metnase expression.

FIG. 4B shows the purification of VS-tagged Intnase/Gypsy Integrase-1 Protein using an anti-V5 sepharose column and progressive KCl elution washes.

FIG. 5A is an illustration of the 3-D transposase domain of Metnase.

FIG. 5B is an illustration of a 3-D representation of the Intnase transposase domain with an inhibitor docked to it.

FIG. 5C is an higher power illustration of a portion of the Intnase (also GIN-1 or Gypsy Integrase-1) transposase domain protein, showing an inhibitor molecule docked in or coupled with the protein.

FIG. 6 shows endonuclease activity of the purified human Intnase/Gypsy Integrase-1 protein (here Intnase). The human Intnase protein was able to linearize plasmid DNA. The arrows denote the linearized plasmid DNA. Intnase thus exhibits double stranded DNA endonuclease activity.

FIG. 7 further exhibits the DNA endonuclease activity of human Intnase/Gypsy Integrase-1 protein (here GIN-1). The human GIN-1 protein was able to cut 4 nucleotides (nts) from the 3′ end of a single stranded DNA oligonucleotide. The arrows denote the fragment resected. GIN-1 protein thus exhibits single stranded DNA endonuclease activity.

FIG. 8 shows that increasing Intnase/Gypsy Integrase-1 expression (here Intnase293cl8 or Intnase293cl9) increases the recovery of DNA replication after arrest of DNA replication using the cancer chemotherapeutic agent hydroxyurea (HU) as shown by the increase in the fraction of cells in S phase at 8 hrs in flow histograms. FIG. 9A shows the virtual docking studies of elvitegravir binding to the active site of the transposase domain of Metnae.

FIG. 9B shows the virtual docking studies of raltegravir binding to the active site of the transposase domain of Metnase.

FIG. 9C shows the virtual docking studies of elvitegravir binding to the active site of the transposase domain of Intnase/Gypsy Integrase-1.

FIG. 9D shows the virtual docking studies of raltegravir binding to the active site of the transposase domain of Intnase/Gypsy Integrase-1.

FIG. 10 are chemical scaffolds identifying a family of molecules, in accordance with the present invention, for inhibiting transposase repair proteins in cancer cells. Dashed lines represent bonds that may be single bonds or double bonds.

FIG. 11 illustrates examples of a few derivatives according to FIG. 10, using the substituents, but not restricted to them.

FIG. 12 illustrates further examples of derivatives according to FIG. 10.

FIG. 13 is a representation of compounds, in accordance with the present invention, bearing bicyclic and spiro substituents, tricyclic and tetracyclic fused rings.

FIG. 14 sets forth examples of symmetric dimmers, in accordance with the present invention.

FIG. 15 illustrates further examples of molecular inhibitors of cancer cell repair proteins, derived from the scaffolds of FIG. 10.

FIG. 16 is a bar graph showing numbers of colonies of pancreatic cancer cells grown after inoculation with different drugs and combinations of chemical agents.

FIG. 17 is another bar graph showing numbers of colonies of colon cancer cells grown after inoculation with different drugs and combinations of chemical agents.

FIG. 18-33 show the effects of various chemical compounds as otherwise disclosed herein against a leukemia cell line (KG-1) or a small cell lung cancer cell line (CRL5898).

FIG. 34 shows a number of chemical compounds and their activities against pancreatic cancer (BxPC3), leukemia (KG-1) or small cell lung cancer (CLR5898). Note that R₁ is preferably H or a C1-C3 alkyl or cycloalkyl group, preferably a

FIG. 35-37 evidence that Intnase is at least partially responsible for survival rates of cancer cells treated with cancer chemotherapy and/or radiation and that inhibitors of Intnase represent exceptional anti-cancer agents. FIG. 35 shows a colony formation assay of cells that over-express Intnase (here Intnase OE) versus control cells (here pCAPP) performed in the presence of the cancer drug hydroxyurea (here HU), which prevents DNA replication. Cells over-expressing Intnase have an increased survival rate. FIG. 36 shows a colony formation assay indicating that cells that over-express Intnase (here Intnase 3) have an increased survival after exposure to radiation (here IR with dose in Gray, Gy) compared to control cells (here pCAPP). FIG. 37 shows that Intnase repression using siRNA (here Intnase KD) decreases survival to exposure with hydroxyurea compared to control cells (here U6 control).

### SUMMARY OF THE INVENTION

The present invention relates to compounds, pharmaceutical compositions and methods of treating cancer.

In a first aspect, the present invention relates to compounds according to the chemical structure:

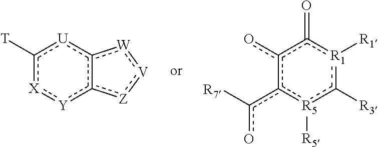

### Where U is a

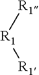

group;

### V is a

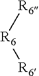

group or a

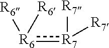

group;

### W is a

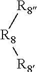

group;

### X is a

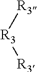

group;

### Y is a

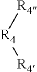

group;

### Z is a

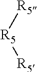

group;
R₁, R₃, R₄, R₅, R₆, R₇ and R₈ are each independently, C, C═O, N, O, S, S═O, or

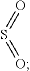

R_{1′}, R_{3′}, R_{4′}, R_{6′}, R_{7′}, and R_{8′} are each independently absent or a C₁-C₆ optionally substituted linear, branched or cyclic alkyl group, a halogen (F, Cl, Br, I), cyano, nitro, nitroso, azido, hydroxyl, thiol, (CH₂)ₙ-aryl which is optionally substituted, (CH₂)ₙ-heterocycle which is optionally substituted, C₁-C₆ optionally substituted alkoxy, (CH₂)ₙ—C₁-C₆ optionally substituted ester, (CH₂)ₙ—C₁-C₆ optionally substituted thioester, C₁-C₆ optionally substituted ether, C₁-C₆ optionally substituted thioether, (CH₂)ₙ—C₁-C₆ optionally substituted acyl (keto) group, (CH₂)ₙ—C₁-C₆ optionally substituted diketo group, (CH₂)ₙ—C₁-C₆ optionally substituted thioacyl (thioketo) group, (CH₂)ₙ—C₁-C₆ optionally substituted carboxylic acid, (CH₂)ₙ—C₁-C₆ optionally substituted thioic acid, (CH₂), —C₁-C₆ optionally substituted sulfone, (CH₂), —C₁-C₆ optionally substituted sulfonate, (CH₂)ₙ—C₁-C₆ optionally substituted sulfate, (CH₂)ₙ—C₁-C₆ optionally substituted sulfoxide, (CH₂)ₙ—C₁-C₆ optionally substituted sulfonamide, (CH₂)ₙ—C₁-C₆ optionally substituted sulfoximide, (CH₂)ₙ—NR¹R² wherein R₁ and R₂ are each independently H, or a C₁-C₃ alkyl group optionally substituted with at least one hydroxyl group; C₁-C₆ optionally substituted diamine, a (CH₂)ₙ-triazene (N—N═N) group which is optionally substituted with one or two C₁-C₆ alkyl groups which are themselves optionally substituted with at least one hydroxyl group, an optionally substituted C₁-C₆ guanidino group, an optionally substituted (CH₂)ₙ-amide group, an optionally substituted (CH₂)ₙ-thioamide group, an optionally substituted (CH₂)ₙ-amidine group, a (CH₂)ₙ-diazo group, an optionally substituted (CH₂)ₙ-diazonium group, an optionally substituted carbamodithioic group, an optionally substituted (CH₂)ₙ-urea group, an optionally substituted (CH₂)ₙ-thiourea group, an optionally substituted (CH₂)ₙ-hydrazine group, an optionally substituted (CH₂)ₙ-hydrazide, an optionally substituted (CH₂)ₙ-isocyanate, an optionally substituted (CH₂)ₙ-thiocyanate, an optionally substituted (CH₂)ₙ-carbonate, an optionally substituted (CH₂)ₙ-carbamate, an optionally substituted (CH₂)ₙ-phosphonate or an optionally substituted (CH₂)ₙ-phosphate, or when R₁, R₃, R₄, R₅, R₆, R₇ or R₈ is a carbon atom, R_{1′} together with R_{1″}, R_{3′} together with R_{3″}, R_{4′} together with R_{4″}, R_{5′} together with R_{5″}, R_{6′} together with R_{6″}, R_{7′} together with R_{7″}, and R_{8′} together with R_{8″} may optionally form an optionally substituted double bond with said carbon atom, or one or more of R_{1′}, R_{3′} and R_{4′}, may optionally form a 5 to 20-membered carbocyclic or heterocyclic ring or fused ring system with T (preferably R^{3′} forms a 5 to 7-membered carbocyclic or heterocyclic ring with T);
R_{1″}, R_{3″}, R_{4″}, R_{5″}, R_{6″}, R_{7″}, and R_{8″} are each independently absent, a C₁-C₁₀ optionally substituted hydrocarbon group, preferably an optionally substituted C₁-C₆ linear, branched or cyclic alkyl group, a halogen (F, Cl, Br, I), cyano, nitro, nitroso, azido, hydroxyl, thiol, (CH₂)ₙ-heterocycle which is optionally substituted, C₁-C₆ optionally substituted alkoxy, (CH₂)ₙ—C₁-C₆ optionally substituted ester, (CH₂)ₙ—C₁-C₆ optionally substituted thioester, C₁-C₆ optionally substituted ether, C₁-C₆ optionally substituted thioether, (CH₂)ₙ—C₁-C₆ optionally substituted acyl (keto) group, (CH₂)ₙ—C₁-C₆ optionally substituted diketo group, (CH₂)ₙ—C₁-C₆ optionally substituted thioacyl (thioketo) group, (CH₂)ₙ—C₁-C₆ optionally substituted carboxylic acid, (CH₂)ₙ—NR¹R² wherein R₁ and R₂ are each independently H, or a C₁-C₃ alkyl group optionally substituted with at least one hydroxyl group; C₁-C₆ diamine which is optionally substituted with one or two C₁-C₆ alkyl groups which are themselves optionally substituted with at least one hydroxyl group, a (CH₂)ₙ-triazene (N—N═N) group which is optionally substituted with one or two C₁-C₆ alkyl groups which are themselves optionally substituted with at least one hydroxyl group, an optionally substituted C₁-C₆ guanidino group wherein the terminal amine is optionally substituted one or two C₁-C₆ alkyl groups which are themselves optionally substituted with at least one hydroxyl group, an optionally substituted (CH₂)ₙ-amide group, an optionally substituted (CH₂)ₙ-thioamide group, an optionally substituted (CH₂)ₙ-amidine group, an optionally substituted (CH₂)ₙ-urea group, an optionally substituted (CH₂)ₙ-thiourea group, an optionally substituted (CH₂)ₙ-hydrazine group, an optionally substituted (CH₂)ₙ-hydrazide, an optionally substituted (CH₂)ₙ-carbonate, an optionally substituted (CH₂)ₙ-carbamate, an optionally substituted (CH₂)ₙ-phosphonate, an optionally substituted (CH₂)ₙ-phosphate, or
when R₁, R₃, R₄, R₅, R₆, R₇ or R₈ is a carbon atom, R_{1″} together with R_{1′}, R_{3″} together with R_{3′}, R_{4″} together with R_{4′}, R_{5″} together with R_{5′}, R_{6″} together with R_{6′}, R_{7″} together with R_{7′}, and R_{8″} together with R_{8′} may optionally form a double bond with said carbon atom which is optionally substituted;
T is a O—R_{9′} group, a C(O)OR_{10′} group, a O—C(O)R_{10′} group or forms a 5 to 20-membererd carbocyclic or heterocyclic ring or fused ring system with one or more of R_{1′}, R_{3′} and R_{4′} (preferably T forms a 5 to 7-membered carbocyclic or heterocyclic ring with R^{3′});
R_{9′} is a C₁-C₆ hydrocarbon, preferably a linear branched or cyclic alkyl group which is optionally substituted, a (CH₂)ⱼ—C₁-C₆ ether or thioether group which is optionally substituted, a (CH₂)ⱼ—C₁-C₆ acyl group which is optionally substituted, a (CH₂)ⱼ—NR¹R² group wherein R₁ and R₂ are each independently H, or a C₁-C₃ alkyl group optionally substituted with at least one hydroxyl group, an optionally substituted (CH₂)ₙ-amide group, an optionally substituted (CH₂)ₙ-thioamide group, an optionally substituted (CH₂)ₙ-aryl group or an optionally substituted (CH₂)ₙ-heterocyclic group;
R_{10′} is a C₁-C₆ hydrocarbon, preferably a linear branched or cyclic alkyl group which is optionally substituted, a (CH₂)ⱼ—C₁-C₆ ether or thioether group which is optionally substituted, a (CH₂)ⱼ—C₁-C₆ acyl group which is optionally substituted, a (CH₂)ⱼ—NR¹R² group wherein R₁ and R₂ are each independently H, or a C₁-C₃ alkyl group optionally substituted with at least one hydroxyl group, an optionally substituted (CH₂)ₙ-amide group, an optionally substituted (CH₂)ₙ-thioamide group, an optionally substituted (CH₂)ₙ-aryl group or an optionally substituted (CH₂)ₙ-heterocyclic group;
j is 1, 2, 3, 4, 5 or 6, preferably 1, 2 or 3;
n is 0, 1, 2, 3, 4, 5, or 6, preferably 0, 1, 2, or 3;
Or a pharmaceutically acceptable salt, solvate or polymorph thereof.

In certain preferred aspects the Metnase inhibitor is according to the chemical structure:

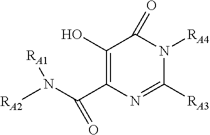

Where R_{A1} is H or a C₁-C₆ alkyl group which is optionally substituted with at least one hydroxyl or halogen group;

### R_{A2} is (1) H;

(2) C₁-C₆ alkyl which is optionally substituted with one or more substituents each of which is independently halogen, —OH, O—C₁-C₆ alkyl, —0-C₁-C₆ haloalkyl, —NO₂, —N(R^{a}R^{b}), —C(═O)R^{a}, —CO₂R^{a}, —SR^{a}, —S(═O)R^{a}, —SO₂R^{a}, or

### —N(R^{a})CO₂R^{b},

(3) C₁-C₆ alkyl which is optionally substituted with one or more substituents each of which is independently halogen, —OH, or O—C₁₋₄ alkyl, and which is substituted with 1 or 2 substituents each of which is independently:
(i) C₃-C₈ cycloalkyl,
(ii) aryl,
(iii) a fused bicyclic carbocycle consisting of a benzene ring fused to a C₅-C₇ cycloalkyl,
(iv) a 5- or 6-membered saturated heterocyclic ring containing from 1 to 4 heteroatoms independently selected from N, O and S,
(v) a 5- or 6-membered heteroaromatic ring containing from 1 to 4 heteroatoms independently selected from N, O and S, or
(vi) a 9- or 10-membered fused bicyclic heterocycle containing from 1 to 4 heteroatoms independently selected from N, O and S, wherein at least one of the rings is aromatic,
(4) C₂-C₅ alkynyl optionally substituted with aryl,
(5) C₃-C₈ cycloalkyl optionally substituted with aryl,
(6) aryl,
(7) a fused bicyclic carbocycle consisting of a benzene ring fused to a C₅-C₇ cycloalkyl,
(8) a 5- or 6-membered saturated heterocyclic ring containing from 1 to 4 heteroatoms independently selected from N, O and S,
(9) a 5- or 6-membered heteroaromatic ring containing from 1 to 4 heteroatoms independently selected from N, O and S, or
(10) a 9- or 10-membered fused bicyclic heterocycle containing from 1 to 4 heteroatoms independently selected from N, O and S, wherein at least one of the rings is aromatic;
wherein each aryl in (2)(ii) or the aryl (3), (4) or (5) or each fused carbocycle in (2)(iii) or the fused carbocycle in (6) is optionally substituted with one or more substituents each of which is independently halogen, —OH, —C₁-C₆ alkyl, —C₁-C₆ alkyl-ORa, —C₁-C₆ haloalkyl, —O—C₁-C₆ alkyl, —O—C1-6 haloalkyl, —CN, —NO₂, —N(RaRb), —C₁-C₆ alkyl-N(R^{a}R^{b}), —C(═O)N(R^{a}R^{b}), —C(═O)R^{a}, —CO₂R^{a}, —C₁-C₆alkyl-CO₂R^{a}, —OCO₂R^{a}, SR^{a}, —S(═O)R^{a}, —SO₂R^{a}, —N(R^{a})SO₂R^{b}, —SO₂N(R^{a}R^{b}), —N(R^{a})C(═O)R^{b}, —N(R^{a})CO₂R^{b}, —C₁-C₆ alkyl-N(R^{a})CO₂R^{b}, aryl, —C₁-C₆ alkyl-aryl, —O-aryl, or —C₀-C₆ alkyl-het wherein het is a 5- or 6-membered heteroaromatic ring containing from 1 to 4 heteroatoms independently selected from N, O and S, and het is optionally fused with a benzene ring, and is further optionally substituted with one or more substituents each of which is independently —C₁-C₆ alkyl, —C₁-C₆ haloalkyl, —O—C₁-C₆ alkyl, —O—C₁-C₆ haloalkyl, oxo (═O), or —CO₂Rₐ; each saturated heterocyclic ring in (2)(iv) or the saturated heterocyclic ring in (7) is optionally substituted with one or more substituents each of which is independently halogen, —C₁-C₆ alkyl, —C₁-C₆ haloalkyl, —O—C₁-C₆ alkyl, —O—C₁-C₆ haloalkyl, oxo, aryl, or a 5- or 6-membered heteroaromatic ring containing from 1 to 4 heteroatoms independently selected from N, O and S; and
each heteroaromatic ring in (2)(v) or the heteroaromatic ring in (8) or each fused bicyclic heterocycle in (2)(vi) or the fused bicyclic heterocycle in (9) is optionally substituted with one or more substituents each of which is independently halogen, —C₁-C₆ alkyl, —C₁-C₆ haloalkyl, —O—C₁-C₆ alkyl, —O—C₁-C₆ haloalkyl, oxo, aryl, or —C₁-C₆ alkyl-aryl; or alternatively R^{a} and R^{b} together with the N to which both are attached form a C₃-C₇ azacycloalkyl which is optionally substituted with one or more substituents each of which is independently —C₁-C₆ alkyl or oxo;
each R^{a}, R^{b}, R^{c}, and R^{d} is independently —H or —C₁-C₆ alkyl which is optionally substituted with at least one hydroxyl group;
R^{k} is a carbocycle or heterocycle, wherein the carbocycle or heterocycle is optionally substituted with one or more substituents each of which is independently
(1) halogen,

### (2) —OH,

### (3) —CN,

(4) —C₁-C₆ alkyl, which is optionally substituted with one or more substituents each of which is independently halogen, —OH, —CN, —O—C₁-C₆ alkyl, —O—C₁-C₆ haloalkyl, —C(═O)R^{a}, —CO₂R^{a}, —SR^{a}, —S(═O)R^{a}, —N(R^{a}R^{b}), —C(═O)—(CH₂)₀₋₂N(R^{a}R^{b}), N(R^{a})—C(═O)—(CH₂)₀₋₂N(R^{b}R^{c}), —SO₂Ra, —N(R^{a})SO₂R^{b}, —SO₂N(R^{a}R^{b}), or N(R^{a})C(R^{b})═O,
(5) —O—C₁-C₆ alkyl, which is optionally substituted with one or more substituents each of which is independently halogen, —OH, —CN, —O—C₁-C₆ alkyl, —O—C₁-C₆ haloalkyl, —C(═O)R^{a}, —CO₂R^{a}, —SR^{a}, —S(═O)R^{a}, —N(R^{a}R^{b}), —C(═O)—(CH₂)₀₋₂N(R^{a}R^{b}), N(R^{a})—C(═O)—(CH₂)₀₋₂N(R^{b}R^{c}), —SO₂R^{a}, —N(R^{a})SO₂R^{b}, —SO₂N(R^{a}R^{b}), or N(R^{a})—C(R^{b})═O,

### (6) —NO₂,

(7) oxo,

### (8) —C(═O)R^{a},

### (9) —CO₂R^{a},

### (10) —SR^{a},

### (11) —S(═O)R^{a},

### (12) —N(R^{a}R^{b}),

### (13) —C(═O)N(R^{a}R^{b}),

(14) —C(═O)—C₁-C₆ alkyl-N(R^{a}R^{b}),

### (15) —N(R^{a})C(═O)R^{b},

### (16) —SO₂R^{a},

### (17) —SO₂N(R^{a}R^{b}),

### (18) —N(R^{a})SO₂R^{b},

### (19) —R^{m},

(20) —C₁-C₆ alkyl-R^{m}, wherein the alkyl is optionally substituted with one or more substituents each of which is independently halogen, —OH, —CN, —C₁-C₆ haloalkyl, —O—C₁-C₆ alkyl, —O—C₁-C₆ haloalkyl, —C(═O)R^{a}, —CO₂R^{a}, —SR^{a}, —S(═O)R^{a}, —N(R^{a}R^{b}), —N(R^{a})CO₂R^{b}, —SO₂Ra, —N(Ra)SO₂Rb, —SO₂N(RaRb), or —N(Ra)—C(Rb)═O,
(21) —C₀-C₆ alkyl-N(R^{a})—C₀-C₆ alkyl-R^{m},
(22) —C₀-C₆ alkyl-O—C₀-C₆ alkyl-R^{m},
(23) —C₀-C₆ alkyl-S—C₀-C₆ alkyl-R^{m},
(24) —C₀-C₆ alkyl-C(═O)—C₁-C₆ alkyl-R^{m},
(25) —C(═O)—O—C₀-C₆ alkyl-R^{m},
(26) —C(═O)N(R^{a})—C₀-C₆ alkyl-R^{m},

### (27) —N(R^{a})C(═O)—R^{m},

(28) —N(R^{a})C(═O)—C₁-C₆ alkyl-R^{m}, wherein the alkyl is optionally substituted with one or more substituents each of which is independently halogen, —OH, —CN, —C₁-C₆ haloalkyl, —O—C₁-C₆ alkyl, —O—C₁-C₆ haloalkyl, —C(═O)R^{a}, —CO₂R^{a}, —SR^{a}, —S(═O)R^{a}, —N(R^{a}R^{b}), —N(R^{a})CO₂R^{b}, —SO₂R^{a}, —N(R^{a})SO₂Rb, —SO₂N(RaR^{b}), or —N(R^{a})—C(R^{b})═O,
(29) —N(R^{a})—C(═O)—N(R^{b})—C₀-C₆ alkyl-R^{m},
(30) —N(R^{a})—C(═O)—O—C₀-C₆ alkyl-R^{m},
(31) —N(R^{a})—C(═O)—N(R^{b})—SO₂—C₀-C₆ alkyl-R^{m},

### (32) —C(═O)—C(═O)—N(R^{a}R^{b}),

(33) —C(═O)—C₁-C₆ alkyl-SO₂R^{a}, or

### (34) —C(═O)—C(═O)R^{m};

wherein the carbocycle in R^{k} is (i) a C₃ to C₈ monocyclic, saturated or unsaturated ring, (ii) a C₇ to C₁₂ bicyclic ring system, or (iii) a C₁₁ to C₁₆ tricyclic ring system, wherein each ring in (ii) or (iii) is independent of or fused to the other ring or rings and each ring is saturated or unsaturated;
the heterocycle in R^{k} is (i) a 4- to 8-membered, saturated or unsaturated monocyclic ring, (ii) a 7- to 12-membered bicyclic ring system, or (iii) an 11 to 16-membered tricyclic ring system; wherein each ring in (ii) or (iii) is independent of or fused to the other ring or rings and each ring is saturated or unsaturated; the monocyclic ring, bicyclic ring system, or tricyclic ring system contains from 1 to 6 heteroatoms selected from N, O and S and a balance of carbon atoms; and wherein any one or more of the nitrogen and sulfur heteroatoms is optionally be oxidized, and any one or more of the nitrogen heteroatoms is optionally quaternized;
each R^{m} is independently a C₃-C₈ cycloalkyl; aryl; a 5- to 8-membered monocyclic heterocycle which is saturated or unsaturated and contains from 1 to 4 heteroatoms independently selected from N, O and S; or a 9- to 10-membered bicyclic heterocycle which is saturated or unsaturated and contains from 1 to 4 heteroatoms independently selected from N, O and S; wherein any one or more of the nitrogen and sulfur heteroatoms in the heterocycle or bicyclic heterocycle is optionally oxidized and any one or more of the nitrogen heteroatoms is optionally quaternized; and wherein
the cycloalkyl or the aryl of R^{m} is optionally substituted with one or more substituents each of which is independently halogen, —C₁-C₆ alkyl optionally substituted with —O—C₁-C₄ alkyl, —C₁-C₆ haloalkyl, —O—C₁-C₆ alkyl, —O—C₁-C₆ haloalkyl, —N(R^{a}R^{b}), aryl, or —C₁-C₆ alkyl-aryl; and
the monocyclic or bicyclic heterocycle defined in Rm is optionally substituted with one or more substituents each of which is independently halogen, —C₁-C₆ alkyl, —C₁-C₆ haloalkyl, —O—C₁-C₆ alkyl, —O—C₁-C₆ haloalkyl, oxo, aryl, —C₁-C₆ alkyl-aryl, —C(═O)-aryl, —CO₂-aryl, —CO₂—C₁-C₆ alkyl-aryl, a 5- or 6-membered saturated heterocyclic ring containing from 1 to 4 heteroatoms independently selected from N, O and S, or a 5- or 6-membered heteroaromatic ring containing from 1 to 4 heteroatoms independently selected from N, O and S; and
each n is independently an integer equal to zero, 1 or 2;

### RA3 is

### (1) —H,

(2) —C₁-C₆ alkyl, which is optionally substituted with one or more substituents each of which is independently halogen, —OH, —CN, —O—C₁-C₆ alkyl, —O—C₁-C₆ haloalkyl, —C(═O)R^{a}, —CO₂R^{a}, —SR^{a}, —S(═O)R^{a}, —N(R^{a}R^{b}), —C(═O)—C₀-C₆ alkyl-N(R^{a}R^{b}), N(R^{a})—C(═O)—C₀-C₆ alkyl-N(R^{b}R^{c}), —SO₂R^{a}, —N(R^{a})SO₂R^{b},

### —SO₂N(R^{a}R^{b}),

### —N(R^{a})—C(═O)R^{b}, or —N(R^{a})C(═O)C(═O)N(R^{a}R^{b}),

### (3) —R^{k},

(4) —C₁-C₆ alkyl-R^{k}, wherein:
(i) the alkyl is optionally substituted with one or more substituents each of which is independently halogen, —OH, —CN, —O—C₁-C₆ alkyl, —O—C₁-C₆ haloalkyl, —N(R^{a}R^{b}), —N(R^{a})CO₂R^{b}, —N(R^{a})C(═O)—C₀-C₆ alkyl-N(R^{b}R^{c}), or —N(R^{a})—C₂-C₆ alkyl-OH with the proviso that the —OH is not attached to the carbon alpha to N(R^{a}); and
(ii) the alkyl is optionally mono-substituted with —R^{S}, —C₁-C₆ alkyl-R^{S}, N(R^{a})—C(═O)—C₀-C₆ alkyl-R^{S}, —N(R^{a})—C₀-C₆ alkyl-R^{S}, —O—C₀-C₆ alkyl-R^{S}, or N(R^{a})—C(═O)—C₀-C₆ alkyl-R^{S};
wherein R^{s} is
(a) aryl which is optionally substituted with one or more substituents each of which is independently halogen, —OH, —C₁-C₆ alkyl, —C₁-C₆ alkyl-OR^{a}, —C₁-C₆ haloalkyl, —O—C₁-C₆ alkyl, —O—C₁-C₆ haloalkyl, methylenedioxy attached to two adjacent carbon atoms, or aryl;
(b) a 4- to 8-membered saturated heterocyclic ring containing from 1 to 4 heteroatoms independently selected from N, O and S; wherein the saturated heterocyclic ring is optionally substituted with one or more substituents each of which is independently halogen, —C₁-C₆C₁₋₆ alkyl, —C₁-C₆ alkyl-OR^{a}, —C₁-C₆ haloalkyl, —O—C₁-C₆ alkyl, —O—C₁-C₆ haloalkyl, —C(═O)R^{a}, —CO₂R^{a}, —C(═O)—C₀-C₆ alkyl-N(R^{a}R^{b}), —SO₂R^{a}, oxo, aryl, or —C₁-C₆ alkyl-aryl; or
(c) a 5- to 7-membered heteroaromatic ring containing from 1 to 4 heteroatoms independently selected from N, O and S; wherein the heteroaromatic ring is optionally substituted with one or more substituents each of which is independently halogen, —C₁-C₆ alkyl, —C₁-C₆ alkyl-OR^{a}, —C₁-C₆ haloalkyl, —O—C₁-C₆ alkyl, —O—C₁-C₆ haloalkyl, oxo, or aryl;
(5) —C₀-C₆ alkyl-O—C₀-C₆ alkyl-R^{k},
(6) —C₀-C₆ alkyl-S(O)ₙ—C₁-C₆ alkyl-R^{k},
(7) —O—C₁-C₆ alkyl-OR^{k},
(8) —O—C₁₋₆ alkyl-O—C₁₋₆ alkyl-R^{k},
(9) —O—C₁₋₆ alkyl-S(O)ₙR^{k},
(10) —C₀₋₆ alkyl-N(R^{a})—R^{k},
(11) —C₀₋₆ alkyl-N(R^{a})—C₁₋₆ alkyl-R^{k},
(12) —C₀₋₆ alkyl-N(R^{a})—C₁₋₆ alkyl-OR^{k},
(13) —C₀₋₆ alkyl-C(═O)—R^{k},
(14) —C₀₋₆ alkyl-C(═O)N(R^{a})—C₀₋₆alkyl-R^{k},
(15) —C₀₋₆ alkyl-N(R^{a})C(═O)—C₀₋₆alkyl-R^{k},
(16) —C₀₋₆ alkyl-N(R^{a})C(═O)—O—C₀₋₆ alkyl-R^{k}, or
(17) —C₀₋₆ alkyl-N(R^{a})C(═O)C(═O)R^{k};

### RA4 is

—C₁₋₆ alkyl which is optionally substituted with one or more substituents each of which is independently
(1) halogen,

### (2) —OH,

### (3) —CN,

(4) —O—C₁₋₆ alkyl,
(5) —O—C₁₋₆ haloalkyl,

### (6) —C(═O)R^{a},

### (7) —CO₂R^{a},

### (8) —SR^{a},

### (9) —S(═O)R^{a},

### (10) —N(R^{a}R^{b}),

### (11) —C(═O)N(R^{a}R^{b}),

(12) —N(R^{a})—C(═O)—C₁₋₆ alkyl-N(R^{b}R^{c}),

### (13) —SO₂R^{a},

### (14) —N(R^{a})SO₂R^{b},

### (15) —SO₂N(R^{a}R^{b}),

### (16) —N(R^{a})—C(R^{b})═O,

(17) —C₃₋₈ cycloalkyl,
(18) aryl,
wherein the aryl is optionally substituted with one or more substituents each of which is independently halogen, —C₁₋₆ alkyl, —C₁₋₆ haloalkyl, —O—C₁₋₆ alkyl, —O—C₁₋₆ haloalkyl, —C₀₋₆alkyl-N(R^{a}R^{b}), or —C₁₋₆ alkyl substituted with a 5- or 6-membered saturated heterocyclic ring containing from 1 to 4 heteroatoms independently selected from N, O and S;
wherein the saturated heterocyclic ring is optionally substituted with from 1 to 3 substituents each of which is independently —C₁₋₆ alkyl, oxo, or a 5- or 6-membered heteroaromatic ring containing from 1 to 4 heteroatoms independently selected from N, O and S; or
(19) a 5- to 8-membered monocyclic heterocycle which is saturated or unsaturated and contains from 1 to 4 heteroatoms independently selected from N, O and S; wherein the heterocycle is optionally substituted with one or more substituents each of which is independently —C₁₋₆alkyl, —O—C₁₋₆ alkyl, oxo, phenyl, or naphthyl; with the proviso that none of the following substituents is attached to the carbon atom in the —C₁₋₆ alkyl group that is attached to the ring nitrogen: halogen, —OH, —O—C₁₋₆ alkyl, —O—C₁₋₆haloalkyl, —SR^{a}, —S(═O)R^{a}, —N(R^{a}R^{b}), or —N(R^{a})—C(R^{b})═O;
Or a pharmaceutically acceptable salt, solvate or polymorph thereof.

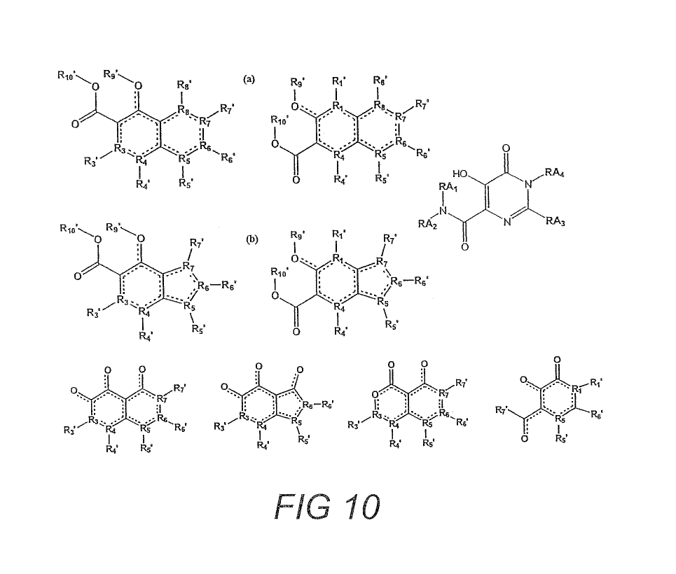

Exemplary structures and compounds of the present invention are presented in the presented application, including FIGS. 10-15 and 34 hereof.

Another aspect of the invention relates to pharmaceutical compositions comprising an effective amount of at least one Metnase and/or Intnase inhibitor as described above, optionally in combination with a pharmaceutically acceptable carrier, additive or excipient, further optionally in combination with an effective amount of an additional bioactive agent, including an anticancer agent, as otherwise described herein.

Another aspect of the invention relates to a method of inhibiting Metnase and/or Intnase wherein Metnase and/or Intnase, and in particular Metnase and/or Intnase in a patient, is exposed to an effective amount of a Metnase and/or Intnase inhibitor as otherwise disclosed herein. Inhibition of Metnase and/or Intnase in a patient, especially a cancer patient (including a cancer patient who is not HIV positive), comprising administering an effective amount of a Metnase and/or Intnase inhibitor to that patient represents another aspect of the present invention.

A further aspect of the invention relates to the use of a Metnase and/or Intnase inhibitor, either alone or preferably in combination with an anticancer agent or other anticancer therapy such as radiation therapy, treat a cancer patient. In this method, a Metnase and/or Intnase inhibitor as otherwise described herein is administered to a cancer patient, alone or in combination with a traditional anticancer agent to treat cancer, especially in an HIV-free (i.e., a patient who is not HIV positive) or an AIDS-free patient. In many aspects of the invention, the combination of a Metnase inhibitor and an anticancer agent or other anticancer therapy provides a synergistically favorable treatment of the cancer.

Still a further aspect of the invention relates to the use of a Metnase and/or Intnase inhibitor, especially including specific Metnase and/or inhibitors described herein for enhance or potentiating the biological effects of an anticancer agent or anticancer therapy (for example as an adjunct to radiation therapy).

### DETAILED DESCRIPTION OF THE INVENTION

The following terms, among others, are used to describe the present invention. It is to be understood that a term which is not specifically defined is to be give a meaning consistent with the use of that term within the context of the present invention as understood by those of ordinary skill.

The term “compound”, as used herein, unless otherwise indicated, refers to any specific chemical compound disclosed herein and includes tautomers, regioisomers, geometric isomers, and where applicable, optical isomers (e.g. enantiomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal and preferably a human, to whom treatment, including prophylactic treatment (prophylaxis), with the compositions according to the present invention is provided. For treatment of those infections, conditions or disease states which are specific for a specific animal such as a human patient, the term patient refers to that specific animal. Metnase and/or Intnase inhibitors according to the present invention may be used to treat cancer per se, or to enhance the effects (potentiate) of other chemotherapeutic agents (anticancer) in treating cancer in patients. However, in certain important aspects of the present invention, metnase and/or intnase inhibitors or antagonists also find use to potentiate the therapeutic effects of radiation therapy.

It is noted that while Metnase and/or Intnase inhibitors according to the present invention may be used to treat cancer in all patients, in certain embodiments, and in particular, with respect to the use of the metnase inhibitors N-(2-(4-(4-fluorobenzylcarbamoyl)-5-hydroxy-1-methyl-6-oxo-1,6-dihydropyrimidine-2-yl)propan-2-yl)-5-methyl-1,3,4-oxadiazole-2-carboxamide (Raltegravir) and (S)-6-(3-chloro-2-fluorobenzyl)-1-(1-hydroxy-3-methylbutan-2-yl)-7-methoxy-4-oxo-1,4-dihydroquinoiline-3-carboxylic acid (Elvitegravir), the use of these compounds to inhibit metnase and/or Intnase and to treat cancer (or potentiate other anticancer agents and/or radiation therapy) in patients other than human immunodeficiency virus HIV positive patients (including patients with acquired immunodeficiency deficiency syndrome (AIDS) and aids-related complex (ARC) is a further aspect of the invention. With respect to patients to whom cancer treatment with Metnase and/or Intnase inhibitors is afforded, such patients are preferably patients other than those to whom HIV therapy and in particular, integrase inhibitors, are to be administered, including patients to whom administration of integrase inhibitors is contraindicated, but who can be treated with Metnase and/or Intnase inhibitors according to the present invention. Thus, the present invention is also used to inhibit metnase and/or intnase, and to treat cancer in patients, other than HIV patients, to whom integrase inhibitors, are administered. In HIV patients, the compounds according to the present invention also may be used to treat cancers other than those cancers which are typically found secondary to HIV infections in HIV patients, in particular, Kaposi's sarcoma, among other cancers, including non-Hodgkin's lymphoma, and invasive cervical cancer. However, other types of cancer also appear to be more common among those infected with HIV. While not classified as AIDS-defining, these malignancies are affecting the HIV/AIDS community greatly and have been referred to as “AIDS-associated malignancies” or “opportunistic” cancers, including Hodgkin's disease, anal cancer, lung cancer and testicular germ cell cancer.

The term “HIV infection” is used to describe an infection in a person with human immunodeficiency virus 1 and/or 2 who is a candidate for treatment with an integrase inhibitor.

The term “human immunodeficieincy virus” or “HIV” shall be used to describe human immunodeficiency viruses 1 and 2 (HIV-1 and HIV-2).

The terms “ARC” and “AIDS” refer to syndromes of the immune system caused by the human immunodeficiency virus, which are characeterized by susceptibility to certain diseases and T cell counts which are depressed compared to normal counts. HIV progresses from Category 1 (Asymptomatic HIV Disease) to Category 2 (ARC), to Category 3 (AIDS), with the severity of the disease.

The term “effective” is used herein, unless otherwise indicated, to describe an amount of a compound or composition which, in context, is used to produce or effect an intended result, whether that result relates to the inhibition of the effects of Metnase and/or Intnase, to potentiate an anticancer agent or radiation therapy in cancer or as otherwise described herein. This term subsumes all other effective amount or effective concentration terms (including the term “therapeutically effective” which are otherwise described in the present application.

The terms “treat”, “treating”, and “treatment”, etc., as used herein, refer to any action providing a benefit to a patient at risk for or afflicted by cancer, including the inhibition of cancer cell or tumor growth, promotion of apoptosis and/or death of cancer cells and/or tumorous tissue resulting in the shrinkage or reduction in cancer cells, including tumors, improvement in the cancer through lessening or suppression of at least one symptom, delay in progression of cancer or the reduction in likelihood or the prevention of metastasis of cancer or the potentiation/enhancement of anticancer agents and/or other cancer therapies including radiation therapy. Treatment, as used herein, encompasses both prophylactic and therapeutic treatment.

The term “Metnase” (also called SETMAR) is used to describe a human protein with a transposase domain derived from Mariner transposons fused to a SET domain. Metnase is expressed in most tissues, methylates histone H3, promotes foreign DNA integration and enhances nonhomologous end joining (NHEJ) of DNA double-strand breaks (DSBs). Metnase is present only in primates, and it possesses partial transposase activity, including sequence-specific DNA binding, assembly of paired end complexes, cleavage of the 5′-end of the Mariner terminal inverted repeat and promotion of integration at a TA dinucleotide target site. Metnase has endonuclease activity that nicks and linearizes but does not degrade supercoiled DNA. Therefore, it is believed that Metnase plays a role in decatenating DNA. DNA replication results in intertwined sister chromatids that must be untangled, or decatenated, to ensure proper chromatid segregation in mitosis and prevent chromatid breaks during anaphase. Metnase interacts with Topoisomerase II alpha (TopoII alpha) and enhances its decatenation activity. It is the target for compounds according to the present invention which exhibit activity as Metnase inhibitors and ultimately DNA repair inhibitors, preferably in cancer cells. In this way, compounds according to the present invention function to enhance anticancer therapy, including anticancer agents and radiation therapy.

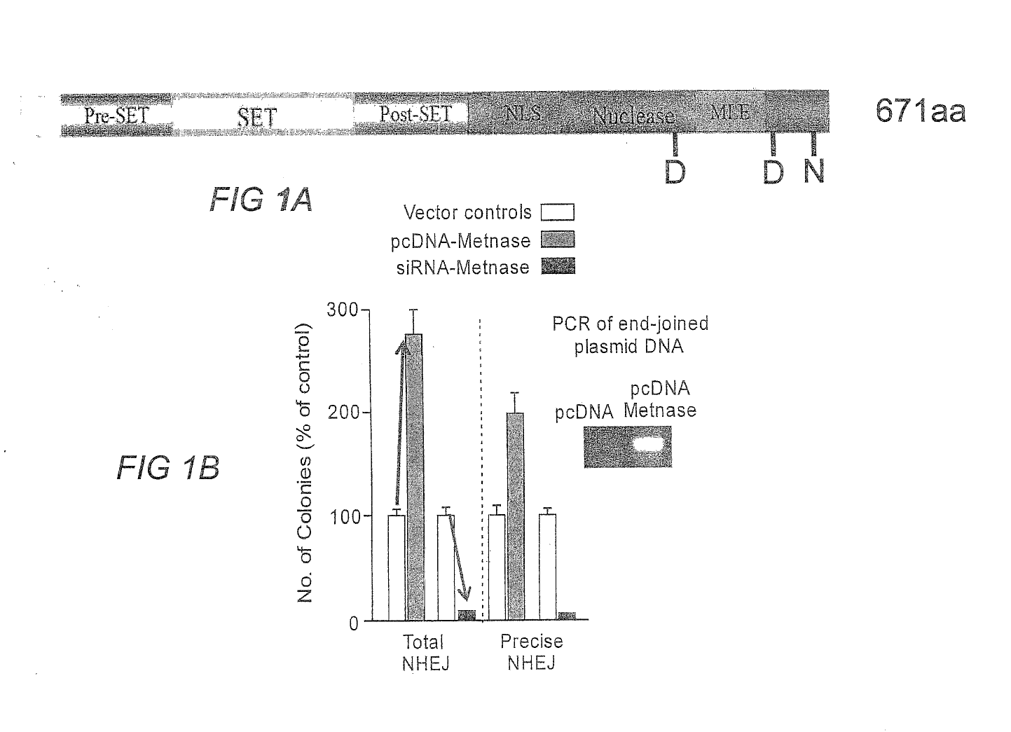

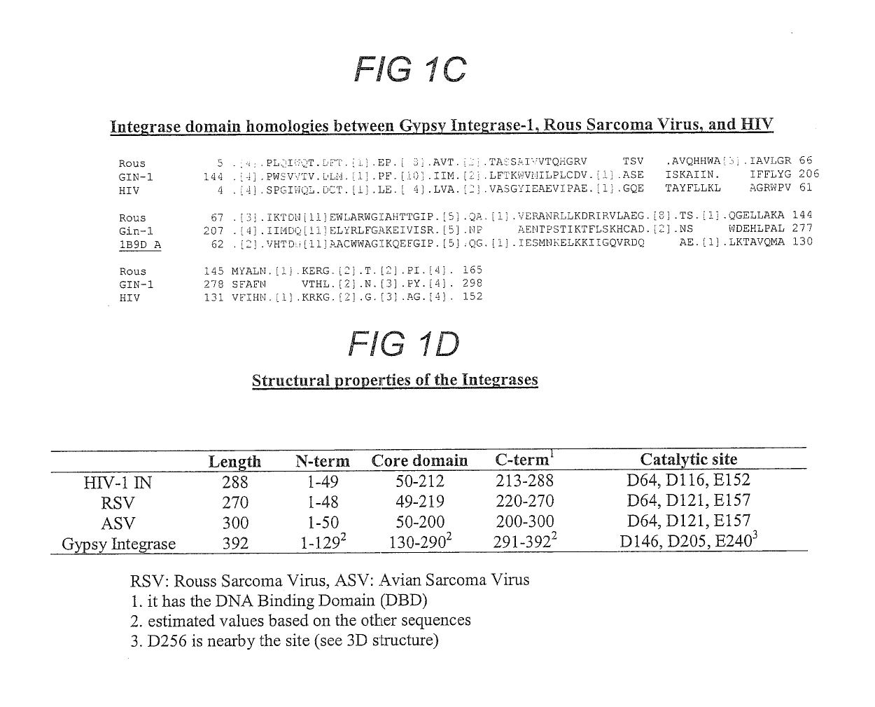

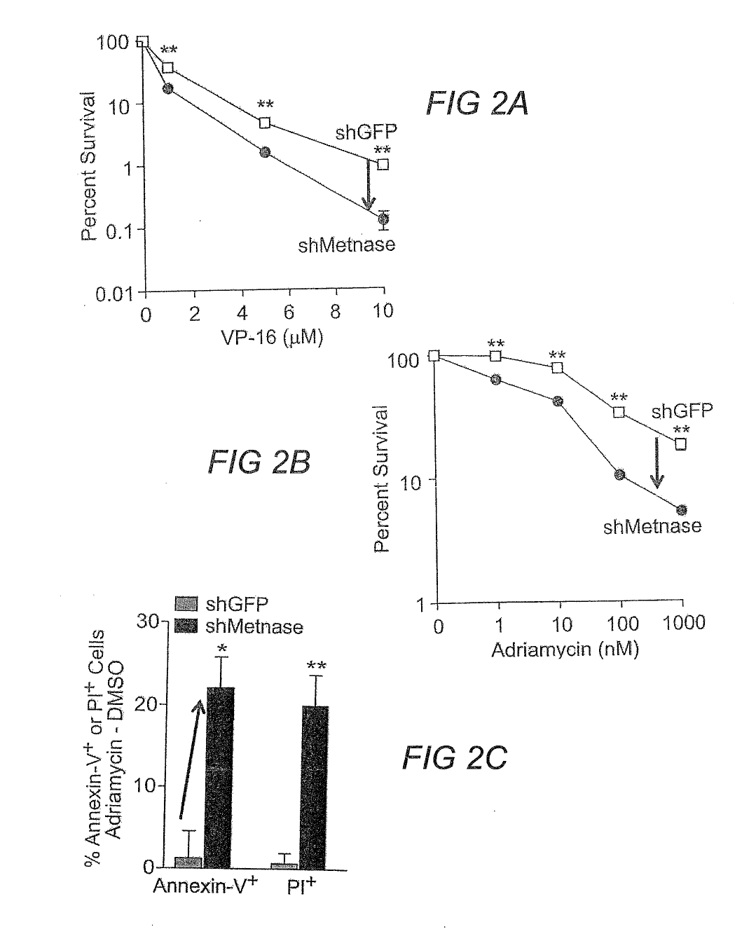

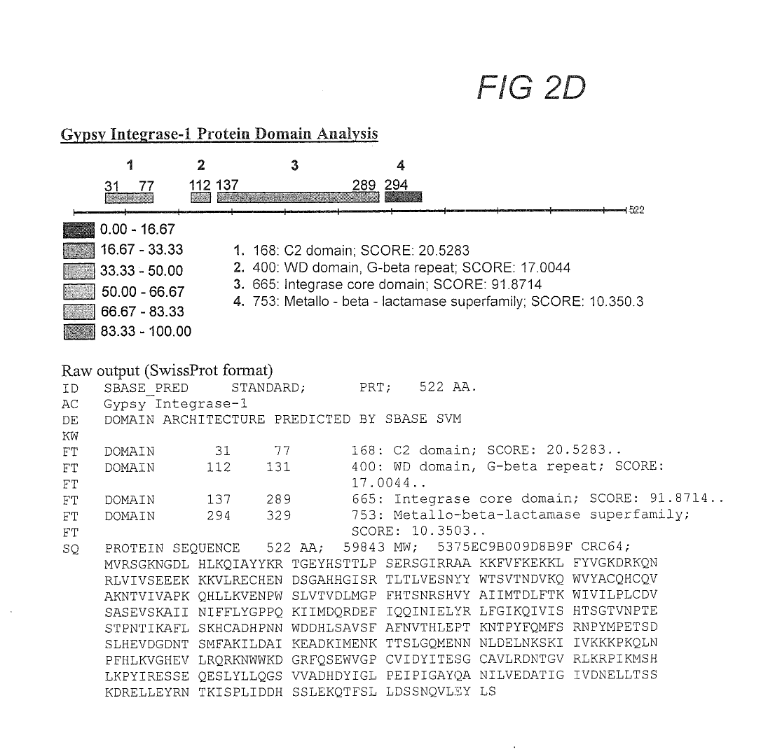

The term “Intnase”, “Gypsy Retransposon Integrase 1”, “GIN-1”, or “Gypsy Integrase-1” is used to describe a human protein with an Integrase domain that is related to HIV Integrase and the retroviral Integrases such as Rous sarcoma virus (FIGS. 1 and 2). Such Integrase domains are known to have endonuclease activity, and it is demonstrated here that Intnase also has such activity, consistent with its membership in the family of proteins. The Intnase protein has endonuclease activity that nicks and linearizes but does not degrade double stranded plasmid DNA. It also has single stranded DNA endonuclease activity, wherein it can specifically cleave off 4 nucleotides from the 3′ end of a single strand of DNA. We demonstrate here that repressing Intnase expression makes cells more sensitive to the cancer chemotherapeutic agent hydroxyurea and over-expressing it reduces sensitivity to hydroxyurea and radiation, both of which are widely used in cancer treatment. This demonstrates that this protein is an important target for inhibition to increase the effectiveness of cancer chemotherapy and radiation. Compounds that bind, or dock, to the active site of the Intnase protein have been identified by computer screening, and these compounds have been demonstrated to increase the sensitivity of several cancer chemotherapeutic agents. In this way, compounds according to the present invention function to enhance anticancer therapy, including anticancer agents and radiation therapy.

The term “Metnase inhibitor” is used to refer to a compound which inhibits Metnase, preferably Metnase in a patient or subject. The term “Intnase inhibitor” is used to refer to a compound which inhibits Intnase, preferably Intnase in a patitent or subject. In certain instances, a compound will inhibit both Metnase and Intnase. A Metnase and/or Intnase inhibitor for use in the present invention includes, for example, compounds which are specifically set forth herein, including the attached figures, as well as compounds which are disclosed in U.S. Pat. Nos. 7,169,780; 7,176,220; 7,435,734, WO2006/060712; U.S. Pat. Nos. 7,538,112; 7,538,112; 7,138,408; 7,517,532; 7,399,763; 7,479,497; 7,148,237; 7,358,249; 7,157,447; 7,368,571; 7,135,467; 7,468,375; 7,135,482; 7,135,482; 7,459,459; 7,115,601; 7,109,201; 7,109,186; 7,037,908; 7,001,912; 7,015,212, each of which patents is incorporated by reference in its entirety herein.

The term “cancer” is used throughout the present invention to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; cutaneous malignancies, myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma and cutaneous malignancies); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991). Representative cancers include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Because of the activity of the present compounds as Metnase inhibitors, the present invention has general applicability treating virtually any cancer in any tissue, thus the compounds, compositions and methods of the present invention are generally applicable to the treatment of cancer.

Although all cancers may be treated with Metnase and/or intnase inhibitors, either alone or in combination with one or more anticancer agents or in combination with radiation therapy, in certain particular aspects of the present invention, the cancer which is treated is pancreatic cancer, lung cancer, breast cancer, leukemia or prostate cancer, including metastatic prostate cancer, especially where radiation therapy is used to treat the prostate cancer. Thus, the present invention is generally applicable and may be used to treat any cancer in any tissue, regardless of etiology.

The term “pharmaceutically acceptable salt” or “salt” is used throughout the specification to describe a salt form of one or more of the compositions herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of prostate cancer, including metastatic prostate cancer, the term “salt” shall mean a pharmaceutically acceptable salt, consistent with the use of the compounds as pharmaceutical agents.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, including sequential administration. Preferably, effective concentrations of all coadministered compounds or compositions are found in the subject at a given time. Metnase inhibitors according to the present invention may be administered with one or more additional anti-cancer agents or other agents which are used to treat or ameliorate the symptoms of cancer, especially prostate cancer, including metastatic prostate cancer. Exemplary anticancer agents which may be coadministered in combination with one or more chimeric compounds according to the present invention include, for example, antimetabolites, inhibitors of topoisomerase I and II, alkylating agents and microtubule inhibitors (e.g., taxol). Specific anticancer compounds for use in the present invention include, for example, adriamyucin aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate; and mixtures thereof, among others.

The term “radiation therapy” is used to describe therapy for cancer, especially including prostate cancer, which may be used in conjunction with the present compounds which exhibit activity as Metnase inhibitors having inherent anticancer activity. Radiation therapy uses high doses of radiation, such as X-rays, to destroy cancer cells. The radiation damages the genetic material of the cells so that they can't grow. Although radiation damages normal cells as well as cancer cells, the normal cells can repair themselves and function, while the cancer cells cannot.

Radiation therapy may be used in combination with the presently claimed compounds, which inhibit Metnase and consequently, the cancer cells' ability to repair damage done by the radiation, thus potentiating radiation therapy. Radiation therapy is most effective in treating cancers that have not spread (metastasized). But it also may be used if the cancer has spread to nearby tissue. Radiation is sometimes used after surgery to destroy any remaining cancer cells and to relieve pain from metastatic cancer.

Radiation is delivered in one of two ways. External-beam radiation therapy and branchytherapy. External-beam radiation therapy uses a large machine to aim a beam of radiation at the tumor. After the area of cancer is identified, an ink tattoo no bigger than a pencil tip is placed on the skin of the subject so that the radiation beam can be aimed at the same spot for each treatment. This helps focus the beam on the cancer to protect nearby healthy tissue from the radiation. External radiation treatments usually are done 5 days a week for 4 to 8 weeks or more. If cancer has spread, shorter periods of treatment may be given to specific areas to relieve pain.

There are basically three types of external radiation therapy: conformal radiotherapy (3D-CRT), intensity-modulation radiation therapy (IMRT) and proton therapy. Conformal radiotherapy uses a three-dimensional planning system to target a strong dose of radiation to the cancer. This helps to protect healthy tissue from radiation. Intensity-modulated radiation therapy uses a carefully adjusted amount of radiation. This protects healthy tissues more than conformal radiotherapy does. Proton therapy uses a different type of energy (protons) than X-rays. This approach allows a higher amount of specifically directed radiation, which protects nearby healthy tissues the most. Sometimes proton therapy is combined with X-ray therapy.

Brachytherapy, or internal radiation therapy, uses dozens of tiny seeds that contain radioactive material. It may be used preferably to treat early-stage prostate and other cancer which is localized. Needles are used to insert the seeds through the skin into tissue, most often the prostate. The surgeon uses ultrasound to locate the tissue and guide the needles. As the needles are pulled out, the seeds are left in place. The seeds release radiation for weeks or months, after which they are no longer radioactive. The radiation in the seeds can't be aimed as accurately as external beams, but they are less likely to damage normal tissue. After the seeds have lost their radioactivity, they become harmless and can stay in place.

Radiation therapy may combine brachytherapy with low-dose external radiation. In other cases, treatment combines surgery with external radiation. In the present invention, compounds which are otherwise claimed may be used as radiation sensitizers to enhance or potentiate the effect of radiation by inhibiting the ability of the cancer tissue to repair the damage done by the radiation therapy.

The terms “antagonist” and “inhibitor” are used interchangeably to refer to an agent, especially including chemical agents which are specifically disclosed herein that decreases or suppresses a biological activity, such as to repress an activity of Metnase.

The term “acyl” is art-recognized and refers to a group represented by the general formula hydrocarbylC(O)—, preferably alkylC(O)—.

The term “acylamino” is art-recognized and refers to a moiety having an amino group and an acyl group and may include substitutents on same as otherwise disclosed herein.

The term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed herein, except where stability of the moiety is prohibitive. For example, substitution of alkenyl groups by one or more alkyl, carbocyclyl, aryl, heterocyclyl, or heteroaryl groups is contemplated.

The terms “alkoxyl” or “alkoxy” as used herein refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ-substituent, where m is 0 to 6 and the substituent is an aryl or substituted aryl group, a cycloalkyl group, a cycloalkenyl, a heterocycle or a polycycle (two or three ringed), each of which may be optionally substituted.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl-substituted alkyl groups. In preferred embodiments, a straight chain or branched chain alkyl has 10 or fewer carbon atoms in its backbone (e.g., C₁-C₁₀ for straight chains, C₁-C₁₀ for branched chains), and more preferably 8 or fewer, and most preferably 6 or fewer. Likewise, preferred cycloalkyls have from 3-10 carbon atoms in their ring structure, and more preferably have 5, 6, 7 or 8 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety or as otherwise described herein. It will be understood by those skilled in the art that the individual substituent chemical moieties can themselves be substituted. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary, non-limiting substituted alkyls are described herein. Cycloalkyls can be further substituted with alkyls, alkenyls, alkynyls, alkoxys, alkylthios, aminoalkyls, carbonyl-substituted alkyls, —CF₃, —CN, and the like.

Analogous substitutions can be made to alkenyl and alkynyl groups to produce, for example, without limitation, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

Unless the number of carbons is otherwise specified, “lower alkyl” as used herein means an alkyl group, as defined above, but having from one to eight carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ-substituent, wherein m is 0 or an integer from 1 to 8 and substituent is the same as defined herein and as otherwise below (R9 and R10 for amine/amino). Representative alkylthio groups include methylthio, ethylthio, and the like.

The terms “amine” and “amino” are art-recognized and refer to both unsubstituted and substituted amines, e.g., a moiety that can be represented, without limitation, by the general formula:

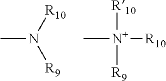

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In certain such embodiments, neither R₉ and R₁₀ is attached to N by a carbonyl, e.g., the amine is not an amide or imide, and the amine is preferably basic, e.g., its conjugate acid has a pKₐ above 7. In even more preferred embodiments, R₉ and R₁₀ (and optionally, R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group. Each of the groups which is bonded to the amine group, where applicable, may be optionally substituted.

The term “amido” is art-recognized as an amino-substituted carbonyl and includes a moiety that can be represented by the general formula:

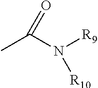

wherein R₉, R₁₀ are as defined above. Preferred embodiments of the amide will not include imides that may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring or aromatic groups containing from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles”, “heteroaromatics” or “heteroaryl groups”. The aromatic ring can be substituted at one or more ring positions with such substituents as otherwise described herein, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon.

The term “carbonyl” is art-recognized and includes such moieties as can be represented by the general formula:

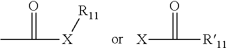

wherein X is a bond or represents an oxygen or a sulfur, and R₁₁ represents, for example without limitation, a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as otherwise described herein without limitation. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The term “electron withdrawing group” refers to chemical groups which withdraw electron density from the atom or group of atoms to which electron withdrawing group is attached. The withdrawal of electron density includes withdrawal both by inductive and by delocalization/resonance effects. Examples of electron withdrawing groups attached to aromatic rings include perhaloalkyl groups, such as trifluoromethyl, halogens, azides, carbonyl containing groups such as acyl groups, cyano groups, and imine containing groups.

The term “ester”, as used herein, refers to a group —C(O)O-substituent wherein the substituent represents, for example, a hydrocarbyl or other substitutent as is otherwise described herein.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, without limitation, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above without limitation, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like, and as otherwise described herein.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include up to 20-membered polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures (which can be cyclic, bicyclic or a fused ring system), preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “5- to 20-membered heterocyclic group” or “5- to 14-membered heterocyclic group” as used throughout the present specification refers to an aromatic or non-aromatic cyclic group having 5 to 20 atoms, preferably 5 to 14 atoms forming the cyclic ring(s) and including at least one hetero atom such as nitrogen, sulfur or oxygen among the atoms forming the cyclic ring, which is a “5 to 20-membered, preferably 5- to 14-membered aromatic heterocyclic group” (also, “heteroaryl” or “heteroaromatic”) in the former case and a “5 to 20-membered”, preferably a″5- to 14-membered non-aromatic heterocyclic group” in the latter case. Among the heterocyclic groups which may be mentioned include nitrogen-containing aromatic heterocycles such as pyrrole, pyridine, pyridone, pyridazine, pyrimidine, pyrazine, pyrazole, imidazole, triazole, tetrazole, indole, isoindole, indolizine, purine, indazole, quinoline, isoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, perimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole. As examples of the “5- to 14-membered aromatic heterocyclic group” there may be mentioned preferably, pyridine, triazine, pyridone, pyrimidine, imidazole, indole, quinoline, isoquinoline, quinolizine, phthalazine, naphthyridine, quinazoline, cinnoline, acridine, phenacene, thiophene, benzothiophene, furan, pyran, benzofuran, thiazole, benzthiazole, phenothiazine, pyrrolopyrimidine, furopyridine and thienopyrimidine, more preferably pyridine, thiophene, benzothiophene, thiazole, benzothiazole, quinoline, quinazoline, cinnoline, pyrrolopyrimidine, pyrimidine, furopyridine and thienopyrimidine. The term “heterocyclic group” shall generally refer to 3 to 20-membered heterocyclic groups, preferablyt 3 to 14-membered heterocyclic groups and all subsets of heterocyclic groups (including non-heteroaromatic or heteroaromatic) subsumed under the definition of heterocyclic group are 3 to 20-membered heterocyclic groups, preferably 3 to 14-membered heterocyclic groups.

The term “8 to 20-membered heterocyclic group”, or “8 to 14-membered heterocyclic group” refers to an aromatic or non-aromatic fused bicyclic or tricyclic group having 8 to 20, preferably 8 to 14 atoms forming the cyclic rings (two or three rings) and include at least one hetero atom such as nitrogen, sulfur or oxygen among the atoms forming the cyclic rings, which is a “8 to 20-membered”, preferably a “8- to 14-membered aromatic heterocyclic group” (also, “heteroaryl” or “heteroaromatic”) in the former case and a “8 to 20-membered”, preferably a “8- to 14-membered non-aromatic heterocyclic group” in the latter case. “8 to 20-membered heterocyclic groups” and “8 to 14 membered heterocyclic groups” are represented by fused bicyclic, tricyclic and tetracyclic ring structures containing nitrogen atoms such as indole, isoindole, indolizine, purine, indazole, quinoline, isoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, perimidine, phenanthroline, phenacene, benzimidazole, pyrrolopyridine, pyrrolopyrimidine and pyridopyrimidine; sulfur-containing aromatic heterocycles such as thiophene and benzothiophene; oxygen-containing aromatic heterocycles such as cyclopentapyran, benzofuran and isobenzofuran; and aromatic heterocycles comprising 2 or more hetero atoms selected from among nitrogen, sulfur and oxygen, such as benzoxazole, benzothiazole, benzothiadiazole, phenothiazine, benzofurazan, phenoxazine, pyrazoloxazole, imidazothiazole, thienofuran, furopyrrole, pyridoxazine, furopyridine, furopyrimidine and thienopyrimidine, among others.

The term “5- to 14-membered non-aromatic heterocyclic group” as used throughout the present specification refers to a non-aromatic cyclic group having 5 to 14 atoms forming the cyclic ring and including at least one hetero atom such as nitrogen, sulfur or oxygen among the atoms forming the cyclic ring. As specific examples there may be mentioned non-aromatic heterocycles such as pyrrolidinyl, pyrrolinyl, piperidinyl, piperazinyl, N-methylpiperazinyl, imidazolinyl, pyrazolidinyl, imidazolidinyl, morpholinyl, tetrahydropyranyl, azetidinyl, oxetanyl, oxathiolanyl, pyridone, 2-pyrrolidone, ethyleneurea, 1,3-dioxolane, 1,3-dioxane, 1,4-dioxane, phthalimideandsuccinimide. As examples of the “5- to 14-membered non-aromatic heterocyclic group” there may be mentioned preferably, pyrrolidinyl, piperidinyl and morpholinyl, and more preferably pyrrolidinyl, piperidinyl, morpholinyl and pyrrole.

The term “8- to 14-membered non-aromatic heterocyclic group” as used throughout the present specification refers to a non-aromatic fused cyclic ring system (generally with two or three rings) having 8 to 14 atoms forming the cyclic rings (bicyclic or tricyclic) and including at least one hetero atom such as nitrogen, sulfur or oxygen among the atoms forming the cyclic rings.

The term “5- to 14-membered heterocyclic group” as used throughout the present specification refers to an aromatic or non-aromatic cyclic group having 5 to 14 atoms forming the cyclic ring and including at least one hetero atom such as nitrogen, sulfur or oxygen among the atoms forming the cyclic ring, which is a “5- to 14-membered aromatic heterocyclic group” in the former case and a “5- to 14-membered non-aromatic heterocyclic group” in the latter case. Specific examples of the “5- to 14-membered heterocyclic group” therefore include specific examples of the “5- to 14-membered aromatic heterocyclic group” and specific examples of the “5- to 14-membered non-aromatic heterocyclic group”.

As the “5- to 14-membered heterocyclic group” there may be mentioned preferably pyrrolidinyl, piperidinyl, morpholinyl, pyrrole, pyridine, pyridone, pyrimidine, imidazole, indole, quinoline, isoquinoline, quinolizine, phthalazine, naphthyridine, quinazoline, cinnoline, acridine, phenacene, thiophene, benzothiophene, furan, pyran, benzofuran, thiazole, benzothiazole, phenothiazine and carbostyryl, more preferably pyrrolidinyl, piperidinyl, morpholinyl, pyrrole, pyridine, thiophene, benzothiophene, thiazole, benzothiazole, quinoline, quinazoline, cinnoline and carbostyryl, and even more preferably thiazole, quinoline, quinazoline, cinnoline and carbostyryl, among others.

The term “6- to 14-membered aromatic heterocyclic group” as used throughout the present specification refers to those substituents defined by “5- to 14-membered aromatic heterocyclic group” which have 6 to 14 atoms forming the cyclic ring. As specific examples there may be mentioned pyridine, pyridone, pyrimidine, indole, quinoline, isoquinoline, quinolizine, phthalazine, naphthyridine, quinazoline, cinnoline, acridine, benzothiophene, benzofuran, thiazole, benzothiazole and phenothiazine*. “8 to 14-membered aromatic heterocyclic groups” refer to those substituents or radicals having 8 to 14 atoms forming fused two or three cyclic ring systems. Specific examples include indole, quinoline, isoquinoline, quinolizine, phthalazine, naphthyridine, quinazoline, cinnoline, acridine, benzothiophene, benzofuran, benzothiazole, pyrrolopyrimidine, pyrrolopyrazine, furopyrimidine and phenothiazine, among numerous others.

The term “6- to 14-membered heterocyclic group” as used throughout the present specification refers to those substituents defined by “5- to 14-membered heterocyclic group” which have 6 to 14 atoms forming the cyclic ring(s). As specific examples there may be mentioned piperidinyl, piperazinyl, N-methylpiperazinyl, morpholinyl, tetrahydropyranyl, 1,4-dioxane and phthalimide.

The term “3 to 7-membered heterocyclic group” as used throughout the present specification refers to those heterocyclic substituents which have 3 to 7 atoms forming the cyclic ring, preferably 5 to 6 atoms forming the cyclic ring.

The term “8 to 14-membered heterocyclic group” as used throughout the present specification refers to those substituents defined “8- to 14-membered heterocyclic groups which have 8 to 14 atoms forming the fused cyclic ring system.

The term “heterocyclylalkyl”, as used herein, refers to an alkyl group substituted with a heterocycle group.

The term “hydrocarbyl”, as used herein, refers to an optionally substituted group that is bonded through a carbon atom and typically has at least one carbon-hydrogen bond and a primarily carbon backbone, but may optionally include heteroatoms. Hydrocarbyl groups include, but are not limited to aryl, heteroaryl, carbocycle, heterocycle, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “polycyclyl” or “polycyclic group” refer to two or more rings (e.g., cycloalkyls, cycloalkenyls, cycloalkynyls, aryls and/or heterocyclyls) in which two or more atoms are common to two adjoining rings, e.g., the rings are “fused rings”. Rings that are joined through non-adjacent atoms are termed “bridged” rings. Each of the rings of the polycycle can be substituted with, without limitation, such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic, non-aromatic and inorganic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents (groups) as otherwise described herein, for example, a halogen, a hydroxyl, a carbonyl (such as a carboxyl, an alkoxycarbonyl, a formyl, or an acyl), an ether, a thioether, a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on a moiety or chemical group can themselves be substituted.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. It is acknowledged that the term “unsubstituted” simply refers to a hydrogen substituent or no substituent within the context of the use of the term.

Preferred substituents for use in the present invention include, for example, within context, hydroxyl, carboxyl, cyano (C≡N), nitro (NO₂), halogen (preferably, 1, 2 or 3 halogens, especially on an alkyl, especially a methyl group such as a trifluoromethyl), thiol, alkyl group (preferably, C₁-C₆, more preferably, C₁-C₃), alkoxy group (preferably, C₁-C₆ alkyl or aryl, including phenyl), ether (preferably, C₁-C₆ alkyl or aryl), ester (preferably, C₁-C₆ alkyl or aryl) including alkylene ester (such that attachment is on the alkylene group, rather than at the ester function which is preferably substituted with a C₁-C₆ alkyl or aryl group), thioether (preferably, C₁-C₆ alkyl or aryl) (preferably, C₁-C₆ alkyl or aryl), thioester (preferably, C₁-C₆ alkyl or aryl), halogen (F, Cl, Br, I), nitro or amine (including a five- or six-membered cyclic alkylene amine, including a C₁-C₆ alkyl amine or C₁-C₆ dialkyl amine), alkanol (preferably, C₁-C₆ alkyl or aryl), or alkanoic acid (preferably, C₁-C₆ alkyl or aryl). More preferably, the term “substituted” shall mean within its context of use alkyl, alkoxy, halogen, hydroxyl, carboxylic acid, nitro and amine (including mono- or di-alkyl substituted amines). Any substitutable position in a compound according to the present invention may be substituted in the present invention, but preferably no more than 5, more preferably no more than 3 substituents are present on a single ring or ring system. Preferably, the term “unsubstituted” shall mean substituted with one or more H atoms.

The term “sulfamoyl” is art-recognized and includes a moiety represented by the general formula:

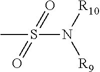

Where R₉ and R₁₀ are substituents as described above.

The term “sulfate” is art-recognized and includes a moiety represented by the general formula:

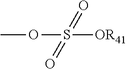

Where R₄₁ is an electron pair, hydrogen, alkyl, cycloalkyl or aryl.

The term “sulfonamido” is art-recognized and includes a moiety represented by the general formula:

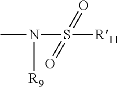

Where R₉ and R′₁₁ are as described above.

The term “sulfonate” is art-recognized and includes a moiety represented by the general formula:

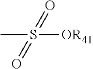

Where R₄₁ is an electron pair, hydrogen, alkyl, cycloalkyl or aryl.

The term “sulfoxido” or “sulfinyl” is art-recognized and includes a moiety represented by the general formula:

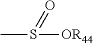

Where R₄₄ is selected from the group consisting of hydrogen, alkyl, alkenyl, alkynyl, cycloalkyl, heterocyclyl, aralkyl or aryl., which groups may be optionally substituted.

The term “thioester” is art-recognized and is used to describe a group —C(O)SR⁹ or —SC(O)R⁹ wherein R⁹ represents an optionally substituted hydrocarbyl group as otherwise described herein.

As used herein, the definition of each expression of alkyl, m, n, etc. when it occurs more than once in any structure, is intended to reflect the independence of the definition of the same expression in the structure.

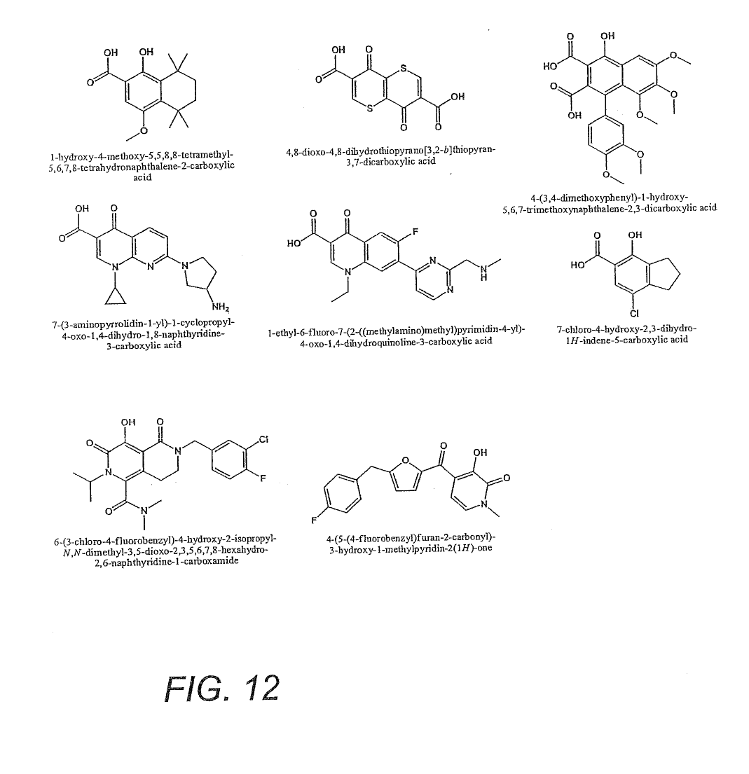

By way of example, certain preferred aromatic and aliphatic rings and their derivatives and substituents which may be used as pharmacophores or substituents in compounds according to the present invention include, but are not limited to, phenyl, benzyl, pyridine, cyclohexadiene, dihydropyridine, tetrahydropyridine, piperidine, pyrazine, tetrahydro-pyrazine, dihydro-pyrazine, piperazine, pyrimidine, dihydro-pyrimidine tetrahydro-pyrimidine, hexahydro-pyrimidine, pyrimidinone, triazine, dihydro-triazine, tetrahydro-triazine, triazinane, tetrazine, dihydro-tetrazine, tetrahydro-tetrazine, tetrazinane, pyrrol, dihydro-pyrrole, pyrrolidine, imidazolidine, dihydro-imidazolidine, imidazole, dihydro-imidazole, azetidine, triazole, dihydro-triazole, triazolidine, tetrazole, dihydro-tetrazole, tetrazolidine, diazepane, tetrahydro-diazepine, dihydro-diazepine, diazepine, oxazole, dihydrooxazole, oxazolidine, isoxazole, dihydroisoxazole, isoxazolidine, thiazole, dihydrothiazole, thiazolidine, isothiazole, dihydroisothiazole, isothiazolidine, oxadiazole, dihydro-oxadiazole, oxadiazolidine, thiadiazole, dihydro-thidiazole, thidiazolidine, oxazinane, dihydro-oxazinane, dihydro-oxazine, oxazine (including morpholine), thiazinane, dihydro-thiazinane, dihydro-thiazine, thiazine (including thiomorpholine), thiazine, furan, dihydrofuran, tetrahydrofuran, thiophene, pyridazine-3,6-dione, tetrahydrothiophene, dihydrothiophene, tetrahydrothiophene, dithiolane, dithiole, dithiolone, dioxolane, dioxole, oxathiole, oxathiolane, pyridinone, dioxane, dioxanedione, benzoquinone, dihydro-dioxine, dioxine, pyran, 3,4-dihydro-2H-pyran, pyranone, 2H-pyran-2,3(4H)-dione, oxathiane, dihydro-oxathiine, oxathiine, oxetane, thietane, thiazeto, cyclohexadienone, lactam, lactone, piperazinone, pyrroledione, cyclopentenone, oxazete, oxazinanone, dioxolane, 3,4-dihydro-2H-thiopyran 1,1-dioxide, dioxolanone, oxazolidinone, oxazolone, thiane 1-oxide, thiazinane 1-oxide, tetrahydro-thiopyran, thiane 1,1-dioxide, dioxazinane, pyrazolone, 1,3-thiazete, thiazinane 1,1-dioxide, 6,7-dihydro-5H-1,4-dioxepine, 1,2-dihydropyridazin-3(4H)-one, pyridine-2,6(1H,3H)-dione, sugar (glucose, mannose, galactose, fucose, fructose, ribose) and derivatives from the compounds which are set forth in attached FIG. 12.

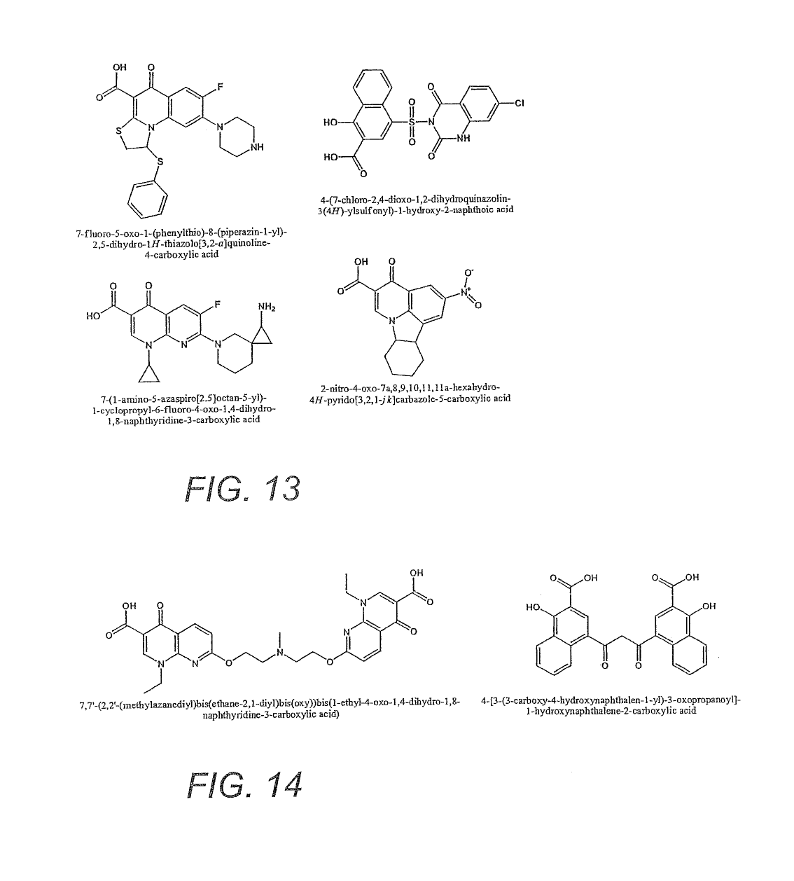

Bicyclic and fused rings include, for example, naphthyl, quinone, quinolinone, dihydroquinoline, tetrahydroquinoline, naphthyridine, quinazoline, dihydroquinazoline, tetrahydroquinazoline, quinoxaline, dihydroquinazoline, tetrahydroquinazoline, pyrazine, quinazoline-2,4(1H,3H)-dione, isoindoline-1,3-dione, octahydro-pyrrolo-pyridine, indoline, isoindoline, hexahydro-indolone, tetrahydropyrrolo oxazolone, hexahydro-2H-pyrrolo[3,4-d]isoxazole, tetrahydro-1,6-naphthyridine, 2,3,4,5,6,7-hexahydro-1H-pyrrolo[3,4-c]pyridine, 1H-benzo[d]imidazole, octahydropyrrolo[3,4-c]pyrrole, 3-azabicyclo[3.1.0]hexane, 7-azabicyclo[2.2.1]hept-2-ene, diazabicyclo-heptane, benzoxazole, indole, 1,4-diazabicyclo[3.3.1]nonane, azabicyclo-octane, naphthalene-1,4-dione, indene, dihydroindene, 2,3,3a,7a-tetrahydro-1H-isoindole, 2,3,3a,4,7,7a-hexahydro-1H-isoindole, 1,3-dihydroisobenzofuran, 1-methyl-3a,4,5,6,7,7a-hexahydro-1H-indole, 3-azabicyclo[4.2.0]octane, 5,6-dihydrobenzo[b]thiophene, 5,6-dihydro-4H-thieno[2,3-b]thiopyran, 3,4-dihydropyrazin-2(1H)-one, 2H-benzo[b][1,4]thiazine, naphthyridin-4(1H)-one, octahydropyrrolo[1,2-a]pyrazine, imidazo-pyridazine, tetrahydroimidazo-pyridazine, tetrahydropyridazine, thiazinone, 5-thia-1-azabicyclo[4.2.0]oct-2-en-8-one, 4-thia-1-azabicyclo[3.2.0]heptan-7-one, 1,6,7,8-tetrahydroimidazo[4,5-d][1,3]diazepine, 8H-thiazolo[4,3-c][1,4]oxazin-4-ium, 8H-thiazolo[4,3-c][1,4]thiazin-4-ium, pteridine, thiazolo[3,4-a]pyrazin-4-ium, 7-(methylimino)-7H-pyrrolo[1,2-c]thiazol-4-ium, thiazolo-pyrazine, 3,7-dioxabicyclo[4.1.0]hept-4-ene, 6,7-dihydro-5H-pyrrolo[1,2-a]imidazole, 3,3a-dihydrofuro[3,2-b]furan-2(6aH)-one, tetrahydro-3aH-[1,3]dioxolo[4,5-c]pyrrole, 7-ethylidene-7H-pyrrolo[1,2-c]thiazol-4-ium, hexahydro-1H-pyrrolo[2,1-c][1,4]oxazine, 6,7,8,8a-tetrahydro-1H-pyrrolo[2,1-c][1,4]oxazine, 2-azabicyclo[2.2.2]oct-2-ene, 6,6a-dihydrothieno[3,2-b]furan-5(3aH)-one, 4,5-dihydropyridin-3(2H)-one, 4,7a-dihydro-3aH-[1,3]dioxolo[4,5-c]pyran, 6,7-dihydro-1H-furo[3,4-c]pyran-1,3(4H)-dione, 3,3a,4,7a-tetrahydro-2H-furo[2,3-b]pyran, 2,4a,7,7a-tetrahydro-1H-cyclopenta[c]pyridine, 4H-pyrano[3,2-b]pyridine-4,8(5H)-dione, 1,2,3,3a,4,7a-hexahydropyrano[4,3-b]pyrrole, 2,3,8,8a-tetrahydroindolizin-7(1H)-one, octahydro-1H-pyrido[1,2-a]pyrazin-1-one, 2,6,7,8,9,9a-hexahydro-1H-pyrido[1,2-a]pyrazin-1-one, 6,7,8,8a-tetrahydropyrrolo[1,2-a]pyrazin-1(2H)-one, hexahydropyrrolo[1,2-a]pyrazin-1(2H)-one, bicyclo[2.2.1]hepta-2,5-diene (FIG. 13).

Spiro moieties: 1,5-dioxaspiro[5.5]undecane, 1,4-dioxaspiro[4.5]decane, 1,4-diazabicyclo[3.2.1]octane, 5-azaspiro[2.5]octane, 5-azaspiro[2.4]heptane, 3,9-diaza-6-azoniaspiro[5.5]undecane, 3,4-dihydrospiro[benzo[b][1,4]oxazine-2,1′-cyclohexane], 7-oxa-4-azaspiro[2.5]oct-5-ene, FIG. 13.

Pharmaceutical compositions comprising combinations of an effective amount of at least one chimeric antibody-recruiting compound according to the present invention, and one or more of the compounds otherwise described herein, all in effective amounts, in combination with a pharmaceutically effective amount of a carrier, additive or excipient, represents a further aspect of the present invention.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin cancers, psoriasis or other diseases which occur in or on the skin. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one additional non-antibody attracting compound which may be used to treat cancer, prostate cancer or metastatic prostate cancer or a secondary effect or condition thereof.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject (e.g. a male human) suffering from cancer can be treated by administering to the patient (subject) an effective amount of a chimeric antibody recruiting compound according to the present invention including pharmaceutically acceptable salts, solvates or polymorphs, thereof optionally in a pharmaceutically acceptable carrier or diluent, either alone, or in combination with other known anticancer or pharmaceutical agents, preferably agents which can assist in treating prostate cancer, including metastatic prostate cancer or ameliorate the secondary effects and conditions associated with prostate cancer. This treatment can also be administered in conjunction with other conventional cancer therapies, such as radiation treatment or surgery.

These compounds can be administered by any appropriate route, for example, orally, parenterally, intravenously, intradermally, subcutaneously, or topically, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-3% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent, as are topically administered compositions.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agents, antibiotics, antifungals, antiinflammatories, or antiviral compounds. In certain preferred aspects of the invention, one or more chimeric antibody-recruiting compound according to the present invention is coadministered with another anticancer agent and/or another bioactive agent, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

The compounds according to the present invention may be prepared using techniques which are well-known in the art. Chemical synthetic approaches are well-known as exemplified in related patents, U.S. Pat. Nos. 7,169,780; 7,538,112; 7,538,112; 7,138,408; 7,517,532; 7,399,763; 7,479,497; 7,148,237; 7,358,249; 7,157,447; 7,368,571; 7,135,467; 7,468,375; 7,135,482; 7,135,482; 7,459,459; 7,115,601; 7,109,201; 7,109,186; 7,037,908; 7,001,912; 7,015,212, each of which patent is incorporated by reference in its entirety herein. The approach uses standard functional group chemistry according to well-known reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the known reaction schemes in the art.

### Metnase

It has been widely believed that transposase family proteins were extinct in humans. In that been discovered, however, that transposase family proteins exist in cancer cells and function as DNA repair proteins or enzymes. Metnase is one such transposase family protein. The existence of Metnase in human cells has been documented by Lee et al., Proc. Natl. Acad. Sci. USA, 102:18075, 2005. FIGS. 1A and 1B show the protein domains of Metnase, and demonstrate its ability to imrove end joining repair of DNA double strand breaks.

Repressing Metnase expression in breast cancer cells has been found to increase the sensitivity of the breast cancer cells to the cancer drugs VP-16 and adriamycin. FIG. 2A is a graph depicting the enhanced action of the cancer drug VP-16 in breast cancer cells conjunction with repression of Metnase expression (shMetnase) compared to control cells (shGFP). FIG. 2B is a graph depicting the enhanced action of the cancer drug adriamycinin in breast cancer cells in conjunction with repression of Metnase expression (shMetnase). FIG. 2C is a bar graph of showing that when Metnase is repressed (shMetnase) there are more apoptotic breast cancer cells (Annexin-5+ or PI+ cells) in the presence of adriamycin compared to control cells (shGFP).

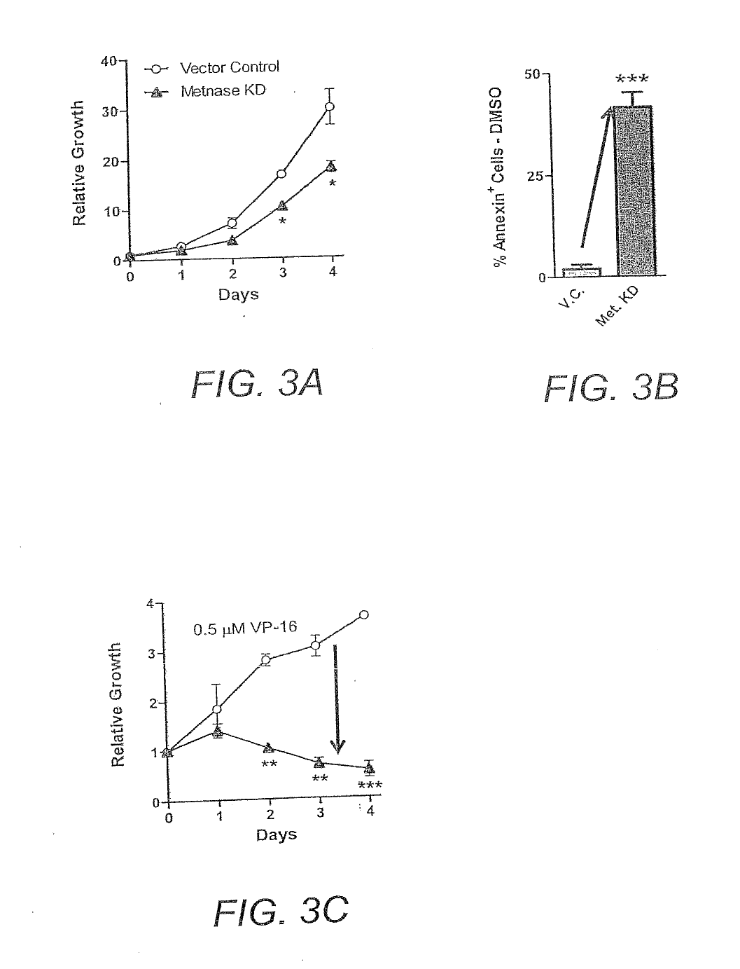

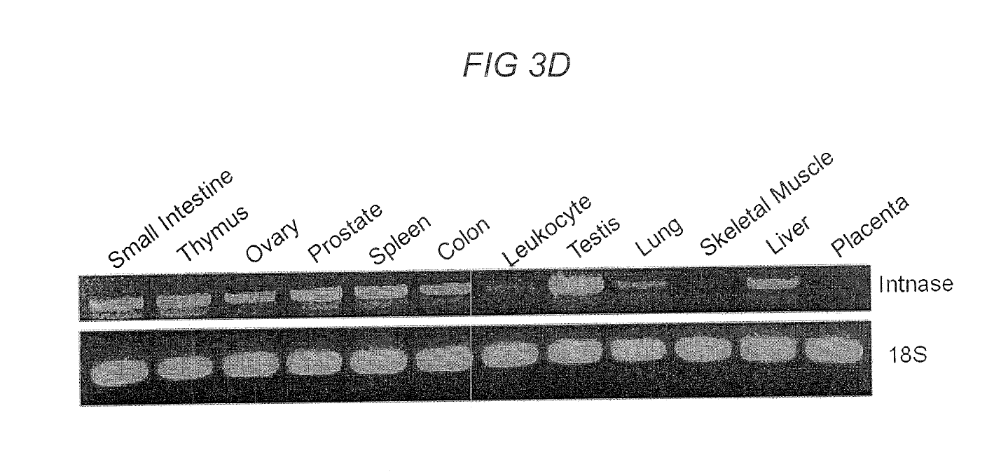

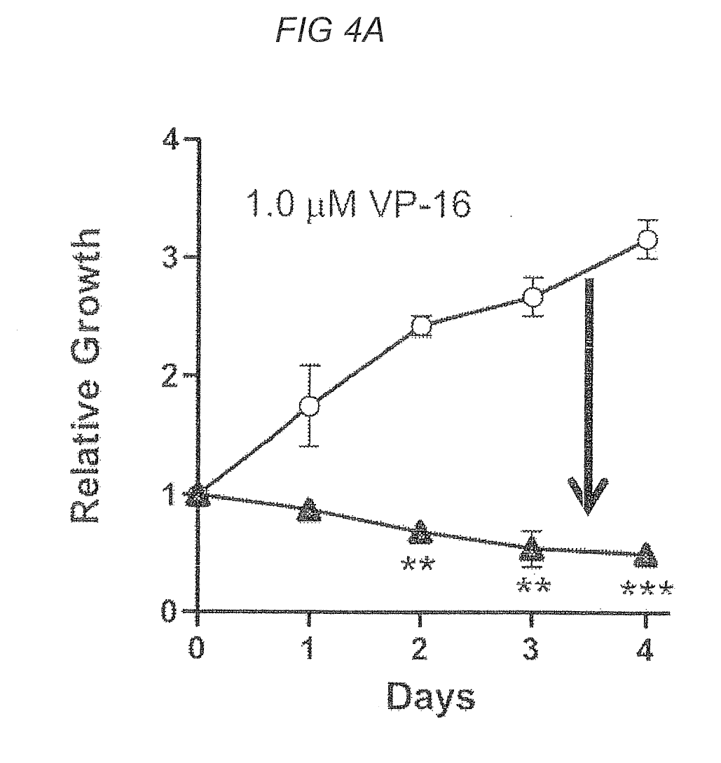

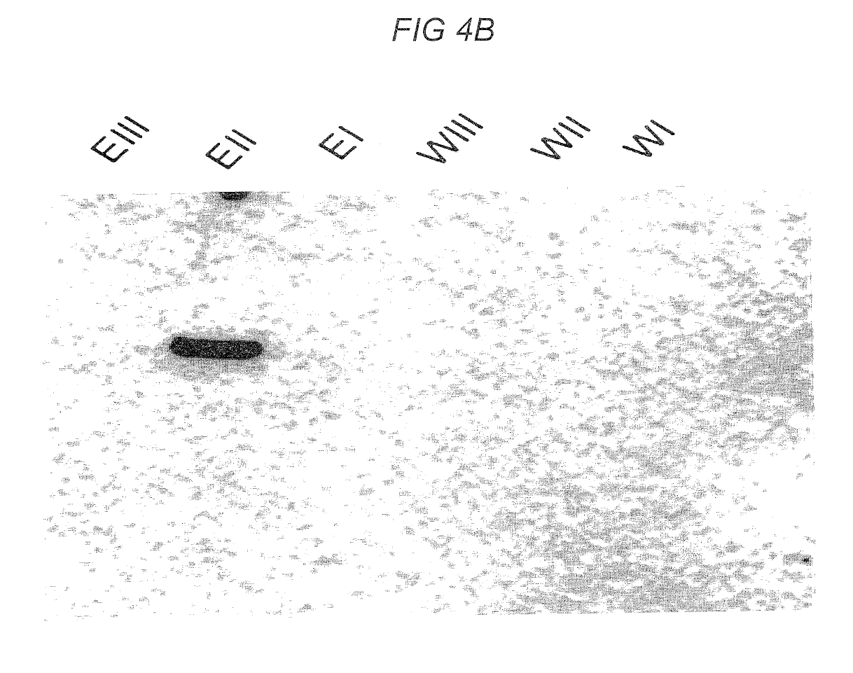

Repressing Metnase expression in leukemia cells has been found to increase the sensitivity of these cancer cells to the cancer drug VP-16. FIG. 3A is a graph showing growth of leukemia cells as a function of normal (vector control) expression levels of Metnase or decreased levels (Metnase KD). FIG. 3B is a bar graph of the increase in apoptotic leukemia cells (Annexin+) after treatment with VP-16 with (Metnase 1(13) or without (V.C.) repression of Metnase. FIG. 3C is a graph showing growth of leukemia cells as a function of time after treatment with 0.5 μM of VP-16 with (closed triangles) or without (open circles) prior repression of Metnase expression. FIG. 4A is a graph showing growth of leukemia cells as a function of time after treatment with 1.0 μM of VP-16 with (closed triangles) or without (open circles) prior repression of Metnase expression.

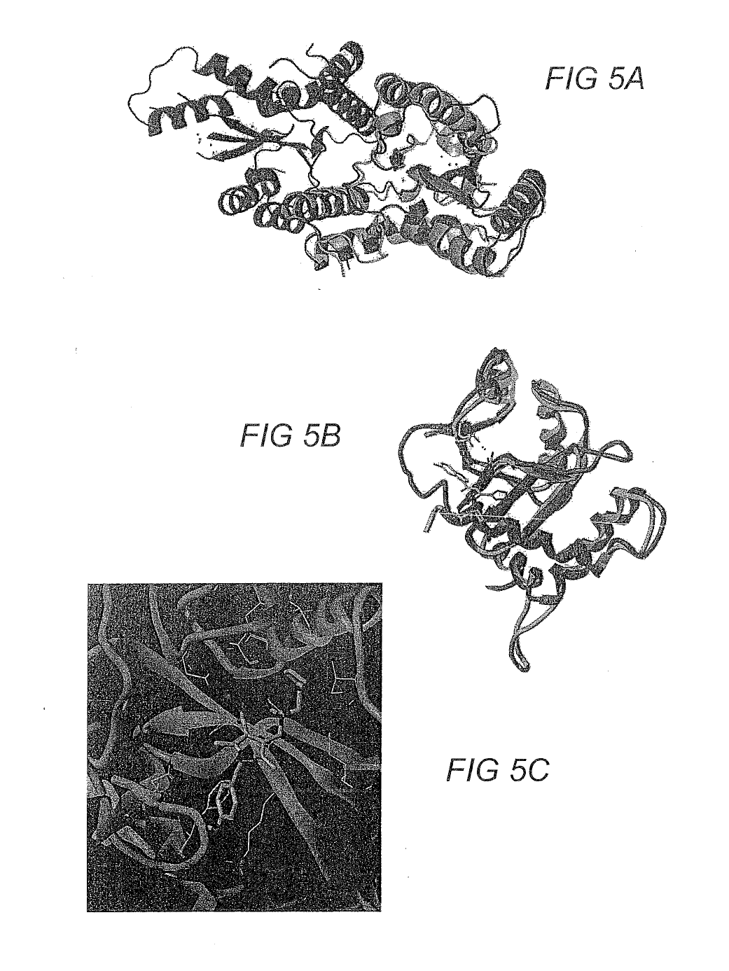

FIGS. 5A and 5B indicate that Metnase and Intnase resemble HIV integrase. The inventors have found a family of small molecules that can dock in or bind to the transposase domain, thereby inhibiting the DNA repair action of the transposase enzymes. This family of molecules shares a common structure and represents a new class of cancer drugs. Among these chemical compositions are two known drugs (one recently FDA approved) used for indications other than cancer treatment.

FIGS. 5B and 5C are graphical illustrations of a portion of the Intnase transposase domain, showing an inhibitor molecule docked in or coupled with the protein so as to inhibit action of the protein.

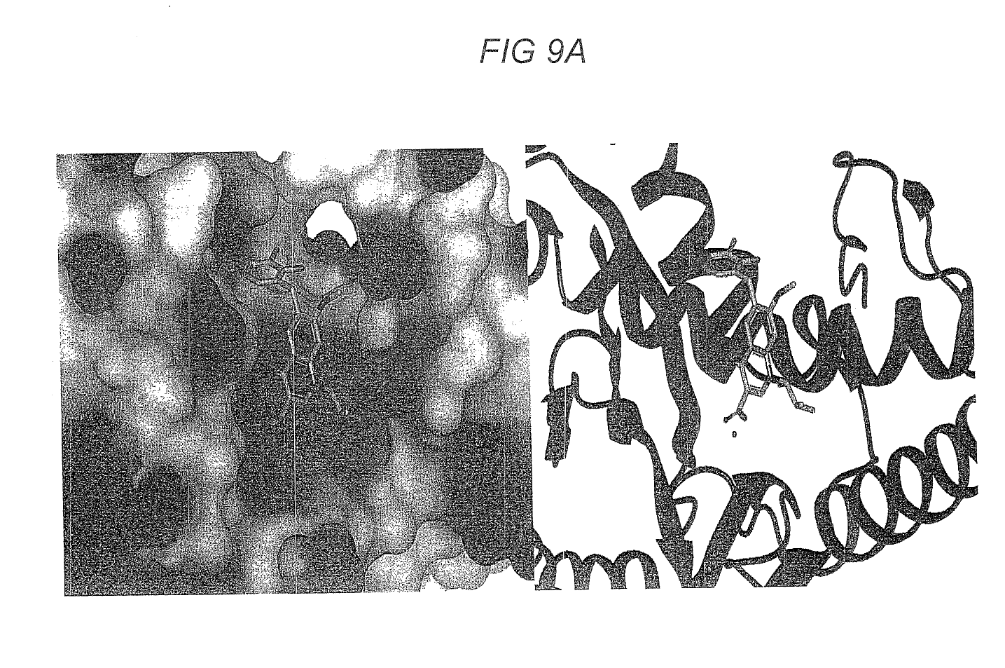

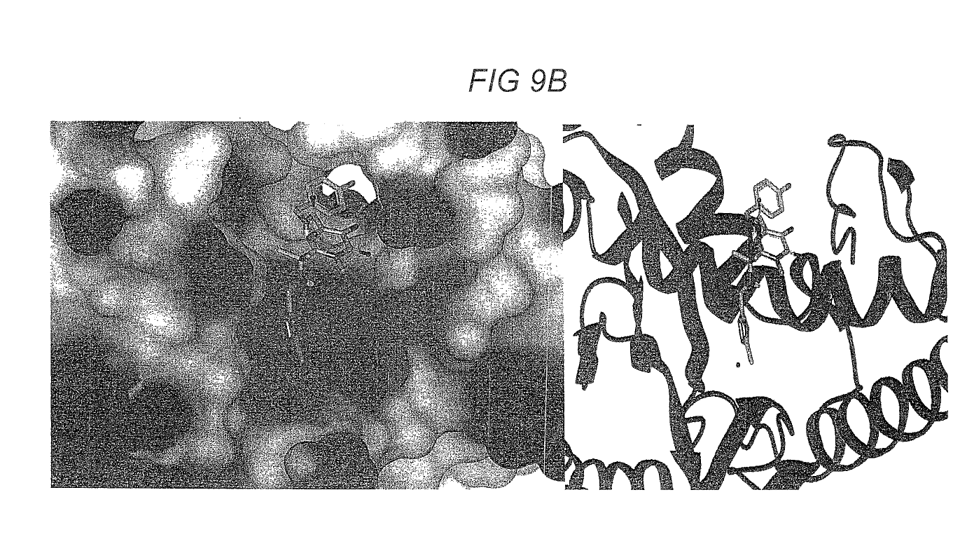

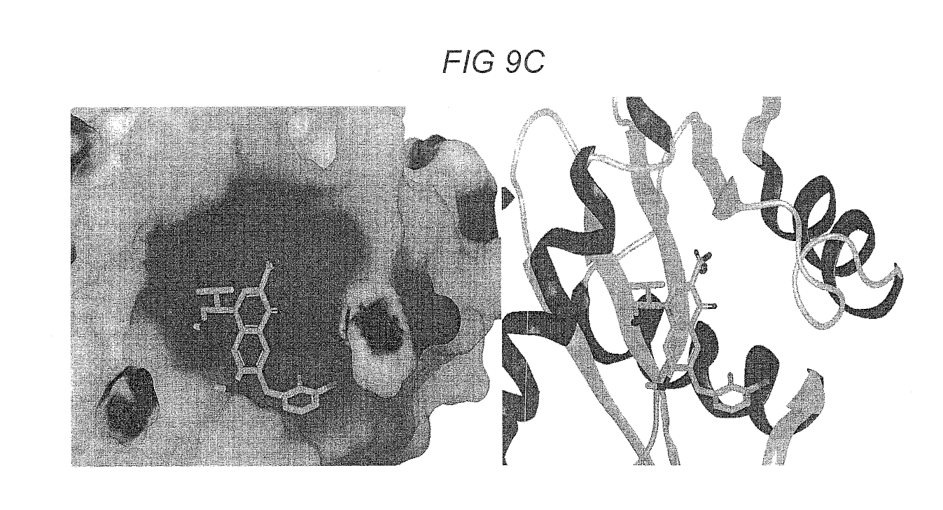

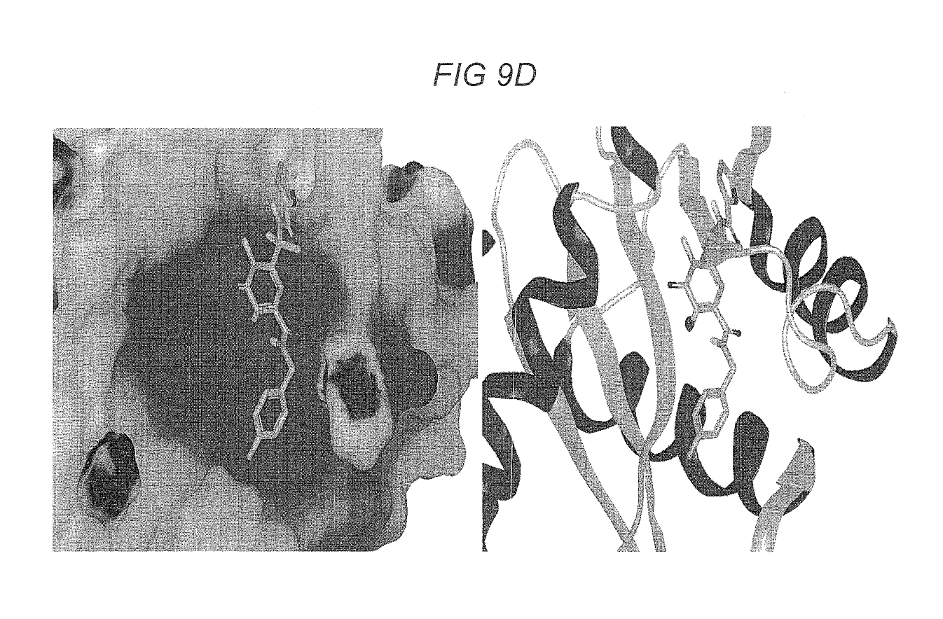

FIG. 9A shows the virtual docking studies of elvitegravir binding to the active site of the transposase domain of Metnase. The left panel shows a molecular surface representation of Metnase's active site with elvitegravir bound to it. Shown are high electron density (red) and low electron density (blue). All atom types are displayed as conventional colors: carbon (gray), hydrogen (white), sulfur (yellow), oxygen (red), nitrogen (blue), halogens (green). Valences are displayed to aromatic rings and double bonds. The right panel shows a cartoon representation of the enzyme (dark blue) with bound elvitegravir, with all atom types are displayed as conventional colors: carbon (gray), hydrogen (white), sulfur (yellow), oxygen (red), nitrogen (blue), halogens (green). Valences are displayed to aromatic rings and double bonds. The pink sphere represents the magnesium ion chelated in the active site.

FIG. 9B shows the virtual docking studies of raltegravir binding to the active site of the transposase domain of Metnase. The left panel shows a molecular surface representation of Metnase's active site with raltegravir bound to it. Shown are high electron density (red) and low electron density (blue). All atom types are displayed as conventional colors: carbon (gray), hydrogen (white), sulfur (yellow), oxygen (red), nitrogen (blue), halogens (green). Valences are displayed to aromatic rings and double bonds. The right panel shows a cartoon representation of the enzyme (dark blue) with bound raltegravir, with all atom types are displayed as conventional colors: carbon (gray), hydrogen (white), sulfur (yellow), oxygen (red), nitrogen (blue), halogens (green). Valences are displayed to aromatic rings and double bonds. The pink sphere represents the magnesium ion chelated in the active site.

### Intnase

It has been widely believed that transposase family proteins were extinct in humans because they might be harmful. However, it has been discovered that transposase family proteins exist in cancer cells and function as DNA repair or replication proteins or enzymes. Intnase/Gypsy Integrase-1 (also GIN-1 or Gypsy Retrotransposon 1) is one such transposase family protein in humans. See FIGS. 1C and 1D, which show the domain homologies and structural properties of Intnase and other integrases, such as Human Immunodeficiency virus-1 (HIV), Rous sarcoma virus (RSV), and Avian sarcoma virus (ASV). Similar or identical amino acids within these shared protein domains are shown in red. FIG. 2D shows the sequence of the human Intnase/Gypsy Integrase-1 protein, and the location of the C2, WD, Integrase, and Metallo-beta-lactamase family domains within the Intnase protein sequence.

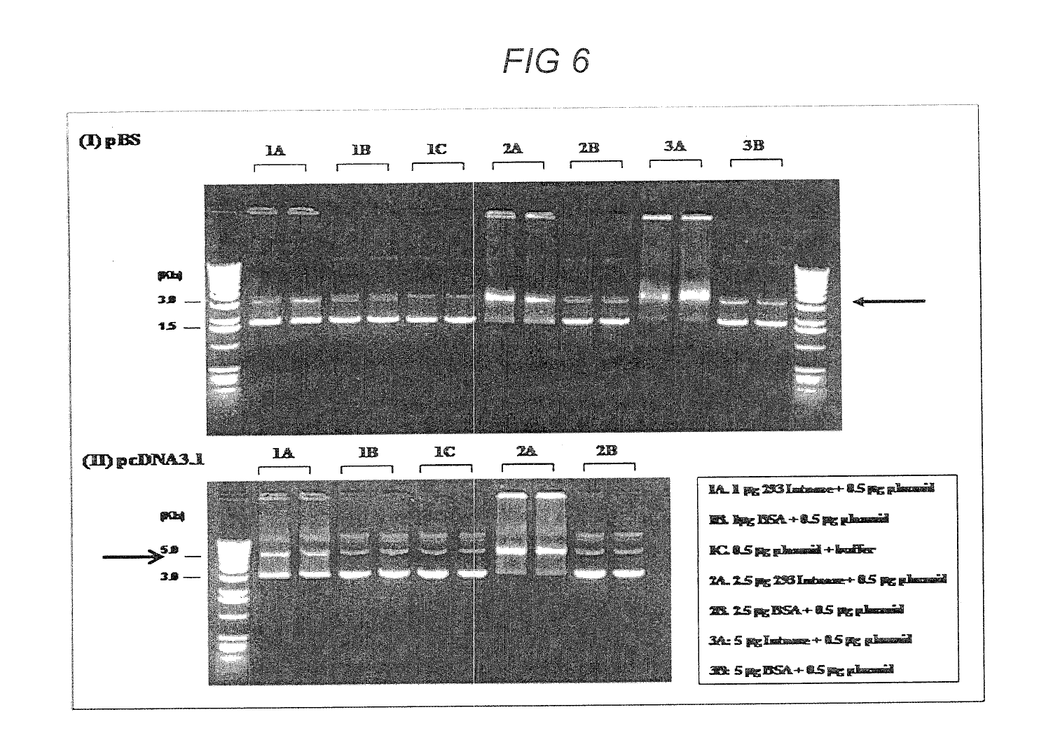

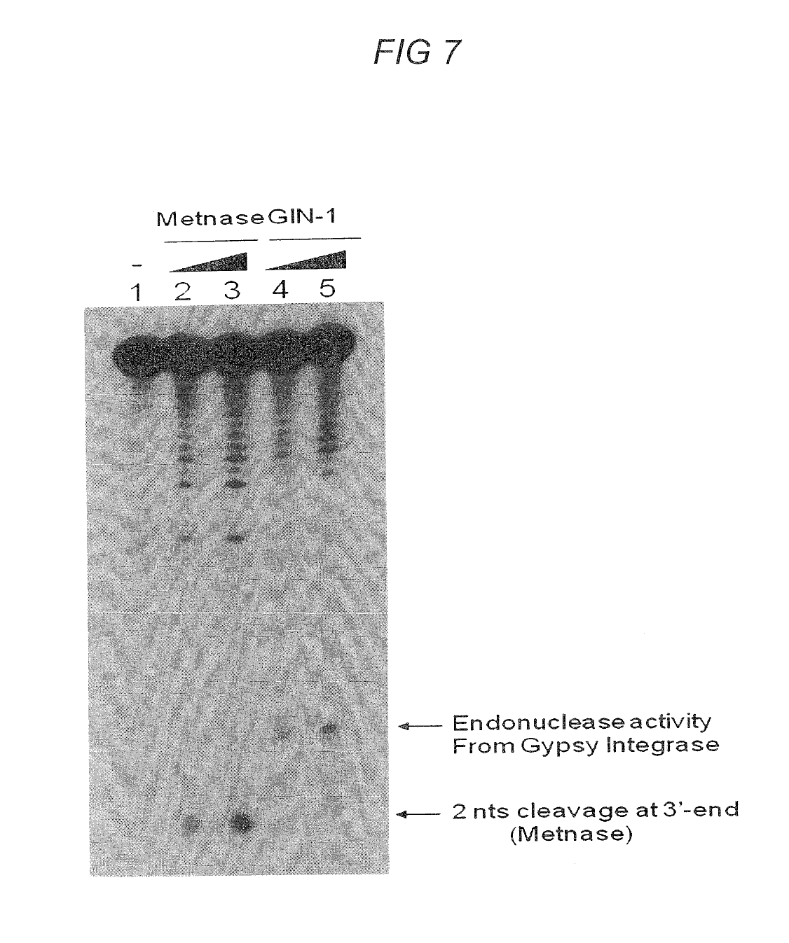

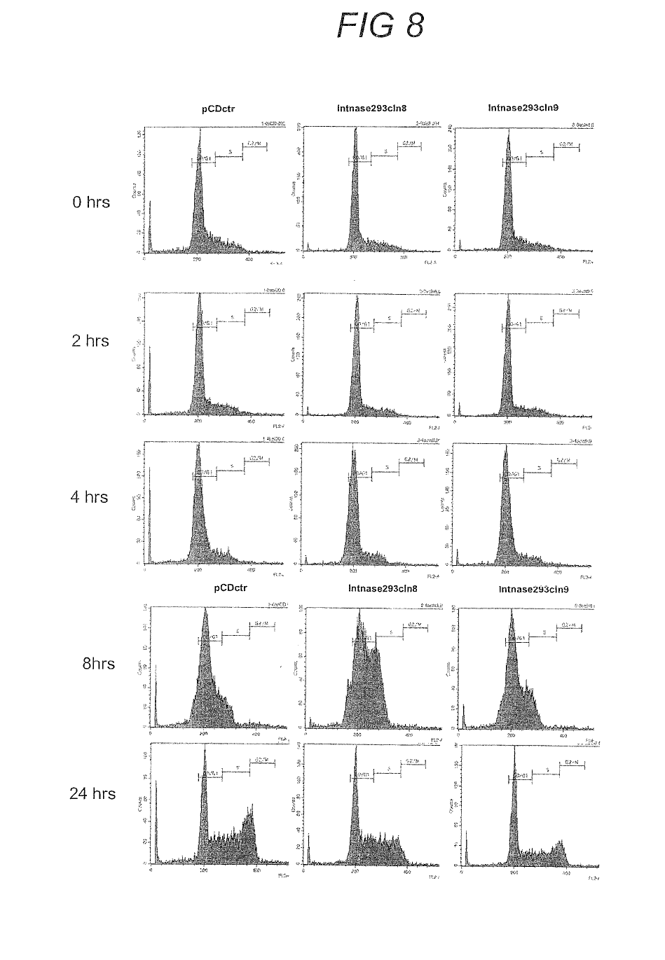

FIG. 3D shows the gene expression analysis using semi-quantitative RT-PCR of Intnase/Gypsy Integrase-1), indicating that this gene is expressed is expressed in virtually all human tissues. FIG. 4B shows a western blot of the isolation of V5-tagged Intnase protein using a anti-V5 column. The protein elutes in fraction II (here EII) from the column, and is the full length, without degradation. This isolated Intnase protein was used in endonuclease assays. Inetgrase proteins were found to have endonucleolytic ability. This was tested in FIGS. 6 and 7, which show that the isolated Intnase protein had both double strand and single strand endonnucleolytic capacity. FIG. 6 demonstrates that the isolated Intnase protein exhibits endonuclease activity on double stranded pBS or pcDNA3.1 plasmid DNA. The increase in linearized plasmid DNA in the presence of the purified isolated Intnase protein is indicated by the arrows. FIG. 7 demonstrates the ability of Intnase (here GIN-1) protein to cleave off 4 nucleotides from the 3′ end of a single strand of radiolabelled DNA. This is compared to the transposase domain protein Metnase protein, which cleaves off 2 nucleotides from the 3′ end of DNA. The dark triangles indicate increasing levels of protein. FIG. 8 shows that Intnase over-expression (designated Intnase293cl8 and cl9) stimulates the recovery from inhibition of DNA replication by the cancer chemotherapeutic agent hydroxyurea compared to control cells (pCDctr). Time in hours is on the left after removal of hydroxyurea. This figure shows the flow cytometric analysis of the cell cycle phase fractions (the first peak represents Go/G1 phases, the second smaller peak G2/M phases, and the population between the two peaks represents S phase) after removal of hydroxyurea. These cell lines were treated with 1 mM hydroxyurea for 18 hours, and then this drug was washed off, and the cells allowed to proceed through the cell cycle. The fraction of cells in cell cycle phases were analyzed by flow cytometry and propidium iodide staining. At 8 hours after removal of the hydroxyurea the over-expressing cells have a higher fraction of cells in S phase, in which cells replicate their DNA, than cells without Intnase over-expression. Since hydroxyurea prevents DNA replication, this indicates that Intnase assists cells in recovery from the cancer drug hydroxyurea and subsequent progression through the cell cycle.

FIGS. 1C, 1D and 2D are evidence that the Intnase protein resembles HIV integrase. FIGS. 6 and 7 shows that Intnase shares some of the biochemical properties of the Integrase family of enzymes, such as endonuclease activity. FIG. 8 shows that over-expression of Intnase helps cells recover from the cancer drug hydroxyurea. These data indicated that Intnase would be an appropriate target for inhibition to increase the effects of cancer treatment. The inventors have found a family of small molecules that can dock within, or bind to, the Integrase domain of Intnase (similar to the related transposase domain protein Metnase), thereby inhibiting the action of Intnase on cancer cell recovery from cancer treatment. This family of molecules shares a common structure and represents a new class of cancer drugs. Among these chemical compositions are two known drugs (one recently FDA approved) used for indications other than cancer treatment.

FIGS. 9C and 9D are illustrations of a 3-dimensional structure of a portion of a Integrase domain of the Intnase protein, showing an inhibitor molecule docked in or coupled with the protein in a manner consistent with inhibition of the protein. The two inhibitors shown are known HIV integrase inhibitors, elvitegravir (FIG. 9C) and raltegravir (FIG. 9D). The left panel shows a molecular surface representation of Intnase's active site with elvitegravir bound to it. Shown are high electron density (red) and low electron density (blue). All atom types are displayed as conventional colors: carbon (gray), hydrogen (white), sulfur (yellow), oxygen (red), nitrogen (blue), halogens (green). Valences are displayed to aromatic rings and double bonds. The right panel shows a cartoon representation of the enzyme (dark blue) with bound elvitegravir, with all atom types are displayed as conventional colors: carbon (gray), hydrogen (white), sulfur (yellow), oxygen (red), nitrogen (blue), halogens (green). Valences are displayed to aromatic rings and double bonds. The pink sphere represents the magnesium ion chelated in the active site.

An exemplary thirty novel chemicals and two known drugs from the family of molecules that bind to or dock with transposase family of enzymes, such as Metnase and/or Intnase here, have been tested for their ability to stop cancer cells from growing.

FIG. 10 are chemical scaffolds identifying a family of molecules for inhibiting transposase repair proteins in cancer cells. Dashed lines represent bonds that may be single bonds or double bonds. These scaffolds comprise derivatives where R₁, R₃, R₄, R₅, R₆, R₇, R₈ can be C, N, O, S, SO, SO₂, C═O.

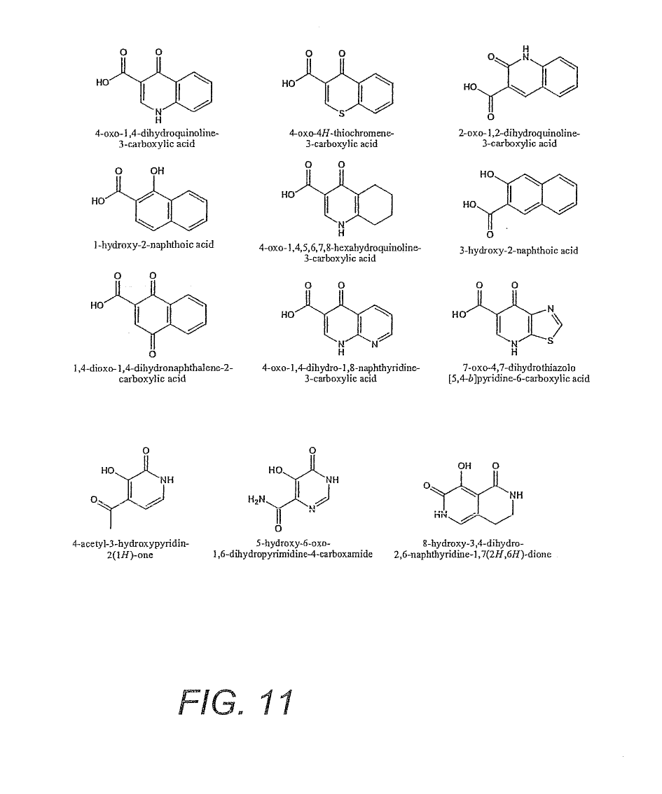

FIG. 11 illustrates examples of a few derivatives according to FIG. 10, using the substituents, but not restricted to them.

By way of example, R₁′, R₃′, R₄′, R₅′, R₆′, R₇′, R₈′ of compounds of the present invention may be represented by methyl, ethyl, propyl, butyl, pentyl, hexyl and their branched, cyclic and their unsaturated alkene, alkyne, diene and halogenated derivatives, halogens (fluorine, chlorine, bromine, iodine), hydroxy, diol, triol, thiol, carboxylic acid, ether, thioether, ester, thioester, ketone, diketo, thioketone, dithioic acid, sulfone, sulfonate, sulfate, sulfoxide, sulfonamide, sulfoximide, amine, diamine, nitrile, nitro, nitroso, azide, triazene, amide, amidine, guanidine, thioamide, diazo, diazonium, carbamodithioic acid, urea, thiourea, hydrazine, hydrazide, isocyanate, thiocyanate, carbonate, carbamate, anhydride, phosphate, and their substituted composites (FIG. 12).

In aspects of the present invention, certain preferred aromatic and aliphatic rings and their derivatives and substituents which may be used as pharmacophores or substituents in compounds according to the present invention include, but are not limited to, phenyl, benzyl, pyridine, cyclohexadiene, dihydropyridine, tetrahydropyridine, piperidine, pyrazine, tetrahydro-pyrazine, dihydro-pyrazine, piperazine, pyrimidine, dihydro-pyrimidine tetrahydro-pyrimidine, hexahydro-pyrimidine, pyrimidinone, triazine, dihydro-triazine, tetrahydro-triazine, triazinane, tetrazine, dihydro-tetrazine, tetrahydro-tetrazine, tetrazinane, pyrrol, dihydro-pyrrole, pyrrolidine, imidazolidine, dihydro-imidazolidine, imidazole, dihydro-imidazole, azetidine, triazole, dihydro-triazole, triazolidine, tetrazole, dihydro-tetrazole, tetrazolidine, diazepane, tetrahydro-diazepine, dihydro-diazepine, diazepine, oxazole, dihydrooxazole, oxazolidine, isoxazole, dihydroisoxazole, isoxazolidine, thiazole, dihydrothiazole, thiazolidine, isothiazole, dihydroisothiazole, isothiazolidine, oxadiazole, dihydro-oxadiazole, oxadiazolidine, thiadiazole, dihydro-thidiazole, thidiazolidine, oxazinane, dihydro-oxazinane, dihydro-oxazine, oxazine (including morpholine), thiazinane, dihydro-thiazinane, dihydro-thiazine, thiazine (including thiomorpholine), thiazine, furan, dihydrofuran, tetrahydrofuran, thiophene, pyridazine-3,6-dione, tetrahydrothiophene, dihydrothiophene, tetrahydrothiophene, dithiolane, dithiole, dithiolone, dioxolane, dioxole, oxathiole, oxathiolane, pyridinone, dioxane, dioxanedione, benzoquinone, dihydro-dioxine, dioxine, pyran, 3,4-dihydro-2H-pyran, pyranone, 2H-pyran-2,3(4H)-dione, oxathiane, dihydro-oxathiine, oxathiine, oxetane, thietane, thiazeto, cyclohexadienone, lactam, lactone, piperazinone, pyrroledione, cyclopentenone, oxazete, oxazinanone, dioxolane, 3,4-dihydro-2H-thiopyran 1,1-dioxide, dioxolanone, oxazolidinone, oxazolone, thiane 1-oxide, thiazinane 1-oxide, tetrahydro-thiopyran, thiane 1,1-dioxide, dioxazinane, pyrazolone, 1,3-thiazete, thiazinane 1,1-dioxide, 6,7-dihydro-5H-1,4-dioxepine, 1,2-dihydropyridazin-3(4H)-one, pyridine-2,6(1H,3H)-dione, sugar (glucose, mannose, galactose, fucose, fructose, ribose) and derivatives from the compounds which are set forth in attached FIG. 12.

FIG. 13 is a representation of compounds, in accordance with the present invention, bearing bicyclic and spiro substituents, tricyclic and tetracyclic fused rings.

Bicyclic and fused rings include, for example, naphthyl, quinone, quinolinone, dihydroquinoline, tetrahydroquinoline, naphthyridine, quinazoline, dihydroquinazoline, tetrahydroquinazoline, quinoxaline, dihydroquinazoline, tetrahydroquinazoline, pyrazine, quinazoline-2,4(1H,3H)-dione, isoindoline-1,3-dione, octahydro-pyrrolo-pyridine, indoline, isoindoline, hexahydro-indolone, tetrahydropyrrolo oxazolone, hexahydro-2H-pyrrolo[3,4-d]isoxazole, tetrahydro-1,6-naphthyridine, 2,3,4,5,6,7-hexahydro-1H-pyrrolo[3,4-c]pyridine, 1H-benzo[d]imidazole, octahydropyrrolo[3,4-c]pyrrole, 3-azabicyclo[3.1.0]hexane, 7-azabicyclo[2.2.1]hept-2-ene, diazabicyclo-heptane, benzoxazole, indole, 1,4-diazabicyclo[3.3.1]nonane, azabicyclo-octane, naphthalene-1,4-dione, indene, dihydroindene, 2,3,3a,7a-tetrahydro-1H-isoindole, 2,3,3a,4,7,7a-hexahydro-1H-isoindole, 1,3-dihydroisobenzofuran, 1-methyl-3a,4,5,6,7,7a-hexahydro-1H-indole, 3-azabicyclo[4.2.0]octane, 5,6-dihydrobenzo[b]thiophene, 5,6-dihydro-4H-thieno[2,3-b]thiopyran, 3,4-dihydropyrazin-2(1H)-one, 2H-benzo[b][1,4]thiazine, naphthyridin-4(1H)-one, octahydropyrrolo[1,2-a]pyrazine, imidazo-pyridazine, tetrahydroimidazo-pyridazine, tetrahydropyridazine, thiazinone, 5-thia-1-azabicyclo[4.2.0]oct-2-en-8-one, 4-thia-1-azabicyclo[3.2.0]heptan-7-one, 1,6,7,8-tetrahydroimidazo[4,5-d][1,3]diazepine, 8H-thiazolo[4,3-c][1,4]oxazin-4-ium, 8H-thiazolo[4,3-c][1,4]thiazin-4-ium, pteridine, thiazolo[3,4-a]pyrazin-4-ium, 7-(methylimino)-7H-pyrrolo[1,2-c]thiazol-4-ium, thiazolo-pyrazine, 3,7-dioxabicyclo[4.1.0]hept-4-ene, 6,7-dihydro-5H-pyrrolo[1,2-a]imidazole, 3,3a-dihydrofuro[3,2-b]furan-2(6aH)-one, tetrahydro-3aH-[1,3]dioxolo[4,5-c]pyrrole, 7-ethylidene-7H-pyrrolo[1,2-c]thiazol-4-ium, hexahydro-1H-pyrrolo[2,1-c][1,4]oxazine, 6,7,8,8a-tetrahydro-1H-pyrrolo[2,1-c][1,4]oxazine, 2-azabicyclo[2.2.2]oct-2-ene, 6,6a-dihydrothieno[3,2-b]furan-5(3aH)-one, 4,5-dihydropyridin-3(2H)-one, 4,7a-dihydro-3aH-[1,3]dioxolo[4,5-c]pyran, 6,7-dihydro-1H-furo[3,4-c]pyran-1,3(4H)-dione, 3,3a,4,7a-tetrahydro-2H-furo[2,3-b]pyran, 2,4a,7,7a-tetrahydro-1H-cyclopenta[c]pyridine, 4H-pyrano[3,2-b]pyridine-4,8(5H)-dione, 1,2,3,3a,4,7a-hexahydropyrano[4,3-b]pyrrole, 2,3,8,8a-tetrahydroindolizin-7(1H)-one, octahydro-1H-pyrido[1,2-a]pyrazin-1-one, 2,6,7,8,9,9a-hexahydro-1H-pyrido[1,2-a]pyrazin-1-one, 6,7,8,8a-tetrahydropyrrolo[1,2-a]pyrazin-1(2H)-one, hexahydropyrrolo[1,2-a]pyrazin-1(2H)-one, bicyclo[2.2.1]hepta-2,5-diene (FIG. 13).

Spiro moieties: 1,5-dioxaspiro[5.5]undecane, 1,4-dioxaspiro[4.5]decane, 1,4-diazabicyclo[3.2.1]octane, 5-azaspiro[2.5]octane, 5-azaspiro[2.4]heptane, 3,9-diaza-6-azoniaspiro[5.5]undecane, 3,4-dihydrospiro[benzo[b][1,4]oxazine-2,1′-cyclohexane], 7-oxa-4-azaspiro[2.5]oct-5-ene, FIG. 13.

The rings described before can be merged to the chemical scaffolds from FIG. 10 in order to provide tricyclic and tetracyclic compounds, as exemplified in FIG. 13. The positions are R₃-R₄, R₄-R₅, R₅-R₆, R₆-R₇, R₇-R₈ and R₃-R₄-R₅, R₅-R₆-R₇.

The substituents previously listed may be used as spacer groups to generate symmetric or asymmetric dimers using the scaffolds in FIG. 10, being one example that shown in FIG. 14. In R₉′, R₁₀′, R₁′, R₃′, R₄′, R₅′, R₆′, R₇′, R₈′: pro-drugs composed by, but not restricted to, carbonate, ester, urea and their dimer derivatives.

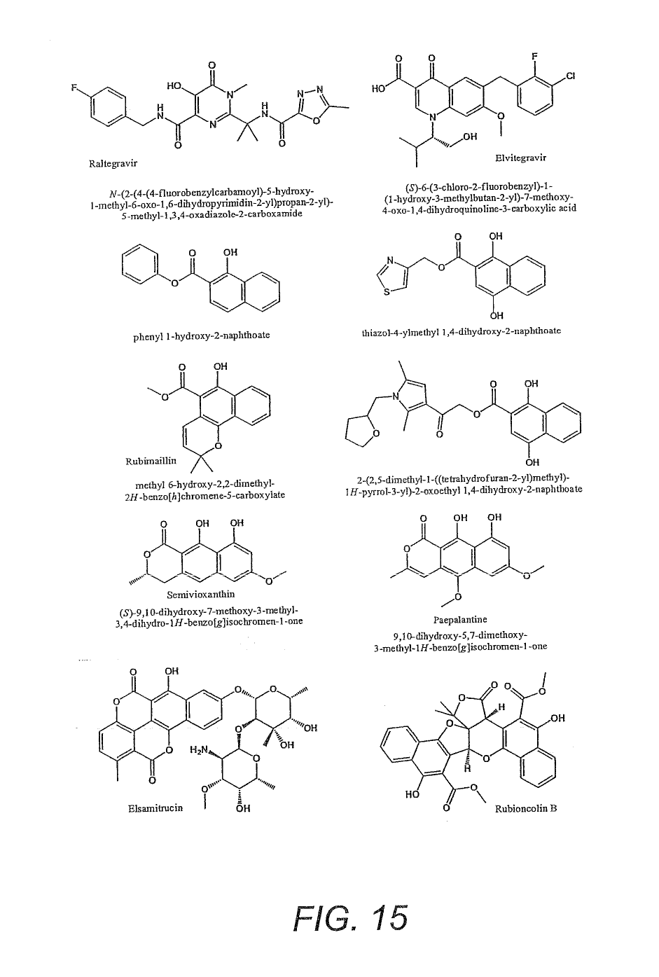

The following discussion pertains to embodiments of inhibitors shown in FIG. 15.

One embodiment set forth in FIG. 15 has the following structure, 6-[(3-chloro-2-fluorophenyl)methyl]-1-[(2S)-1-hydroxy-3-methylbutan-2-yl]-7-methoxy-4-oxoquinoline-3-carboxylic acid.

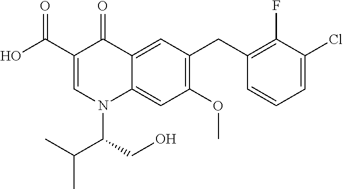

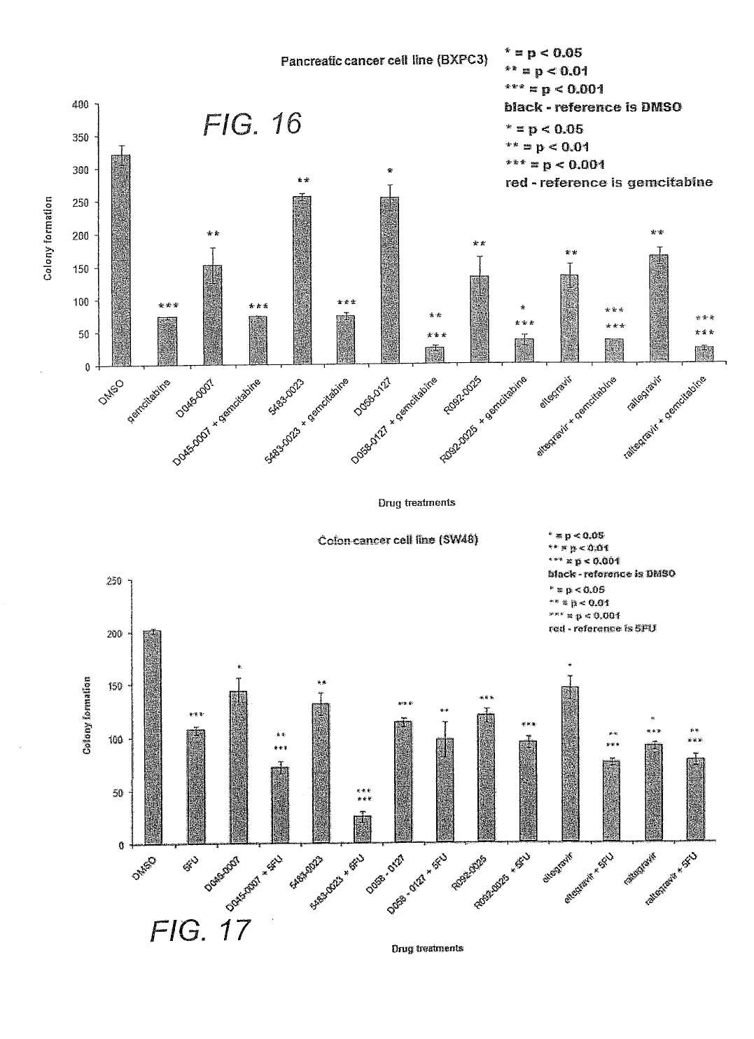

The compound Elvitegravir (JTK-303), shown in FIG. 15, is an HIV integrase strand transfer inhibitor, currently in Phase III clinical trials. Its intended drug target is gag-pol, HIV-1 integrase. It was reported as a moderate CYP3A inducer; serum half-life, 3 hours. Maximum dose administered in humans was 800 mg bid (dose escalation study). This compound was cytotoxic for cancer cells alone and in conjunction with chemotherapy agents (FIGS. 16 and 17).

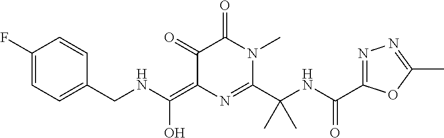

Another embodiment of the present invention shown in FIG. 15 has the following structure, N-[2-[(4Z)-4-[[(4-fluorophenyl)methylamino]-hydroxymethylidene]-1-methyl-5,6-dioxopyrimidin-2-yl]propan-2-yl]-5-methyl-1,3,4-oxadiazole-2-carboxamide is claimed.

This compound, Raltegravir (MK-0518), is an HIV integrase inhibitor, currently FDA approved and launched as Isentress®. Raltegravir has as its intended drug target gag-pol, HIV-1 integrase. Raltegravir inhibits HIV-1 integrase (IC50=10 nM) by acting as integrase strand transfer inhibitor. It does not inhibit human DNA polymerases and HIV-1 reverse transcriptase polymerase activity. In tests conducted, raltegravir did not significantly inhibit any of the 166 enzymes, transporters and receptors included in a screening panel (IC50>10 μM for all assays). Raltegravir is not an inhibitor of ABCB1 (MDR-1; P-gp). Reltegravir is not a substrate, nor an inducer or inhibitor of cytochrome P450 enzymes. Raltegravir is indicated in combination with other anti-retroviral medicinal products for the treatment of human immunodeficiency virus (HIV-1) infection in treatment-experienced adult patients with evidence of HIV-1 replication despite ongoing anti-retroviral therapy. Serum half-life, 9 hours. Maximum recommended daily dose is 800 mg. This compound was cytotoxic for cancer cells alone and in conjunction with chemotherapy agents (FIGS. 16 and 17).

The compositions discussed hereinafter and depicted in FIG. 10-15 are covered by the substructure definitions above and are reported active in some cancer-(or immuno-) related assays, and as such serve as additional examples of the cancer treatment compositions and methods disclosed herein.

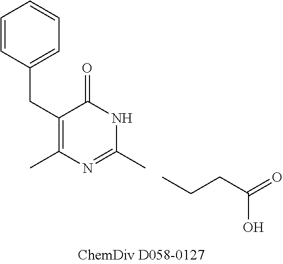

| | |
|---|---|
| Molecular Wt | 304.364 |
| Molecular Formulae | C15H16N2O3S |

This compound was cytotxoic for cancer cells alone and increased the cytotoxicity of chemotherapy when added (FIGS. 16,17,31).

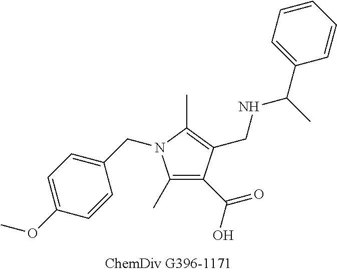

| | |
|---|---|
| MWt | 392.491 |
| MF | C24H28N2O3 |

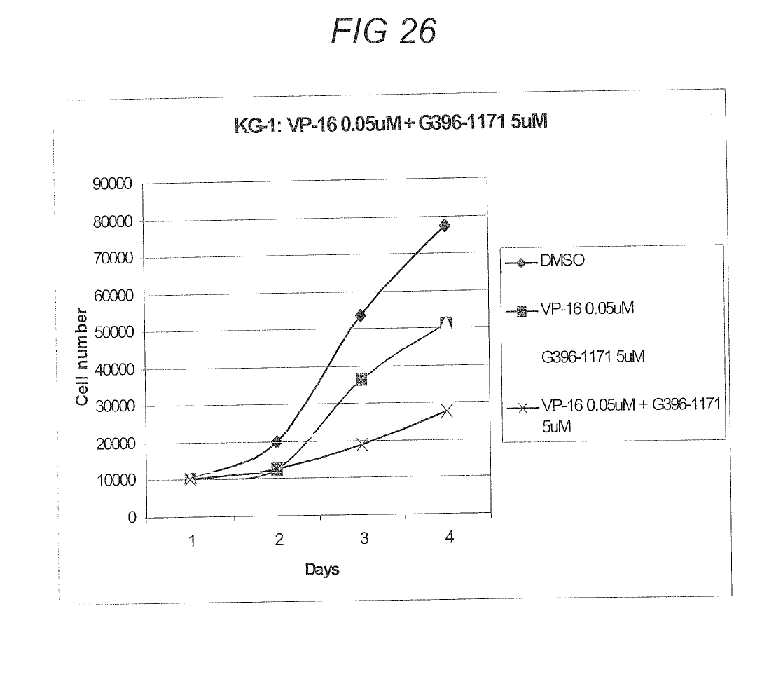

This compound was cytotxoic for cancer cells alone and increased the cytotoxicity of chemotherapy when added (FIGS. 26,27.).

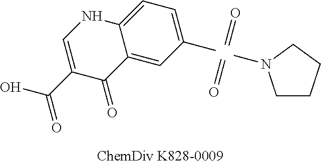

| | |
|---|---|
| MWt | 322.336 |
| MF | C14H14N2O5S |

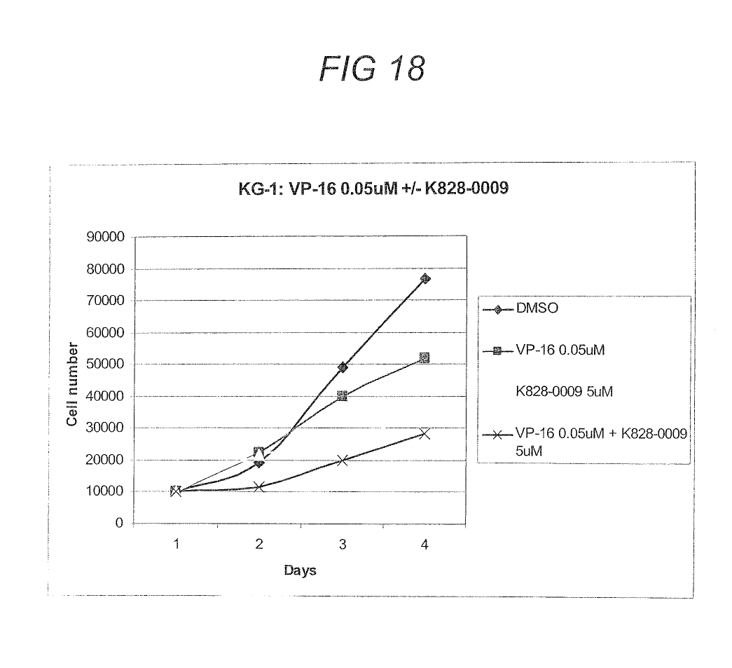

This compound was cytotxoic for cancer cells alone and increased the cytotoxicity of chemotherapy when added (FIG. 18).

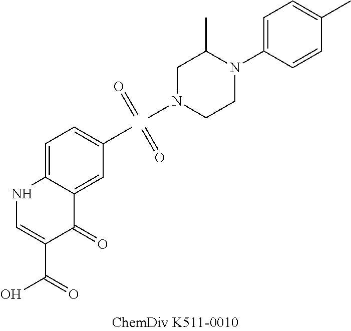

| | |
|---|---|
| MWt | 441.5 |
| MF | C22H23N3O5S |

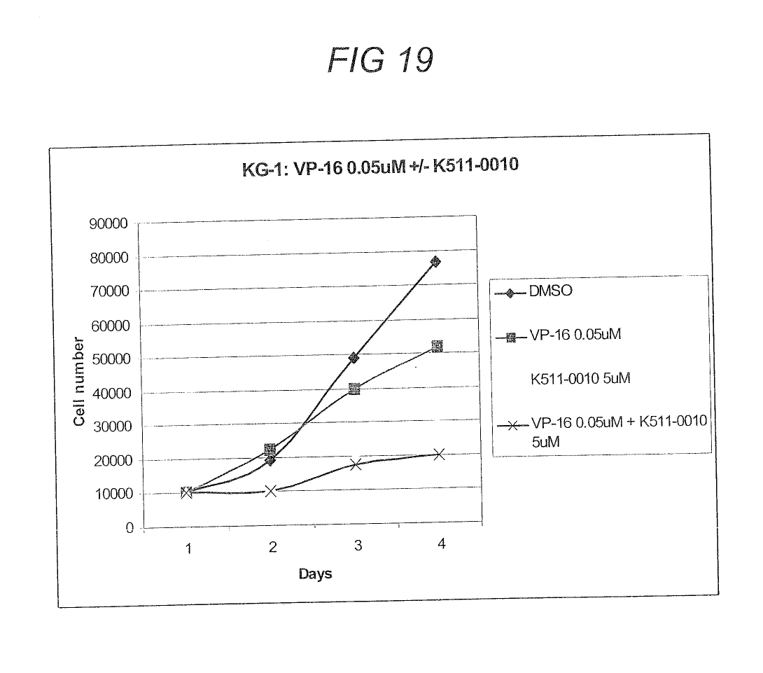

This compound was cytotxoic for cancer cells alone and increased the cytotoxicity of chemotherapy when added (FIG. 19).

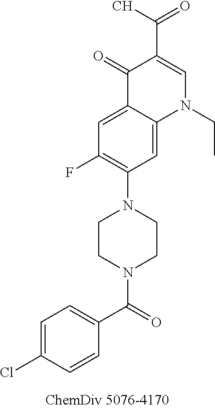

| | |
|---|---|
| MWt | 457.882 |
| MF | C23H21ClFN3O4 |

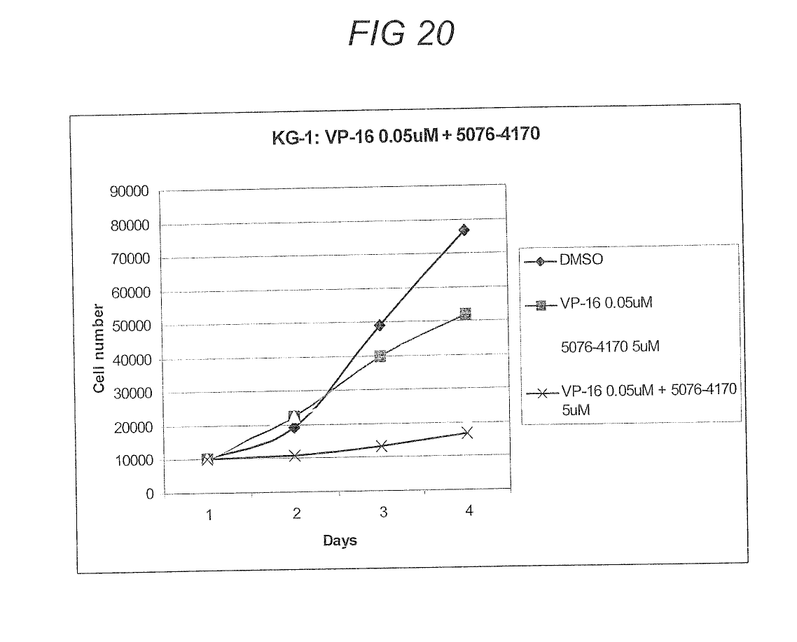

This compound was cytotxoic for cancer cells alone and increased the cytotoxicity of chemotherapy when added (FIGS. 20,21).

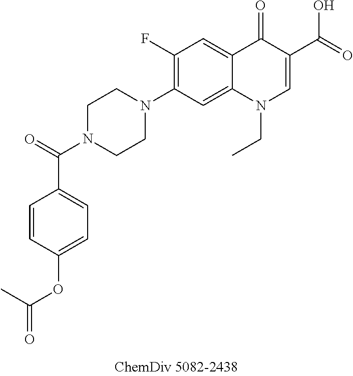

| | |
|---|---|
| MWt | 481.473 |
| MF | C25H24FN3O6 |

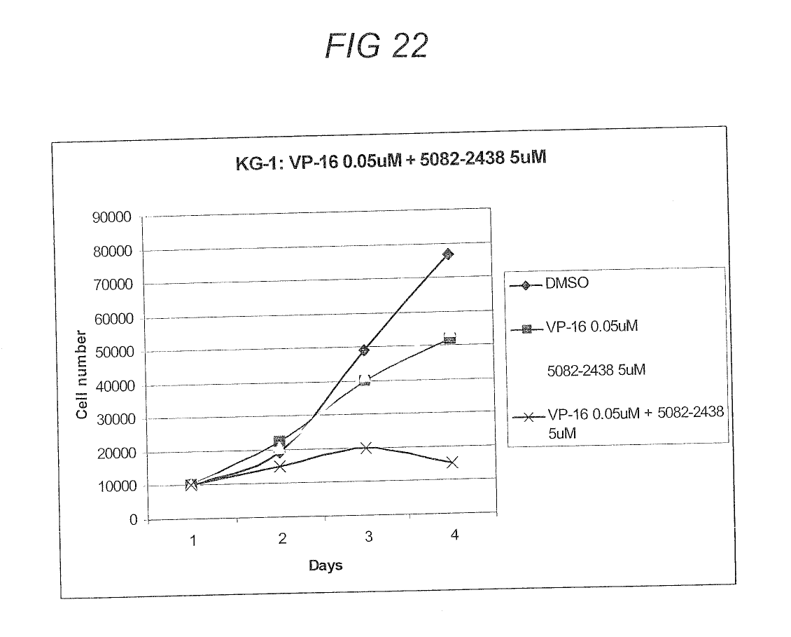

This compound was cytotxoic for cancer cells alone and increased the cytotoxicity of chemotherapy when added (FIGS. 22,23).

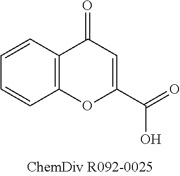

| | |
|---|---|
| MWt | 190.152 |
| MF | C10H6O4 |

This compound was cytotxoic for cancer cells alone and increased the cytotoxicity of chemotherapy when added (FIGS. 16,17).

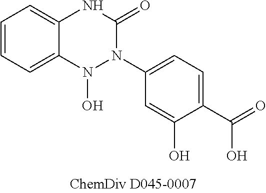

| | |
|---|---|
| MWt | 301.254 |
| MF | C14H11N3O5 |

This compound was cytotxoic for cancer cells alone and increased the cytotoxicity of chemotherapy when added (FIGS. 16,17,32).

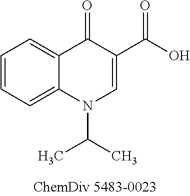

| | |
|---|---|
| MWt | 231.247 |
| MF | C13H13NO3 |

This compound was cytotxoic for cancer cells alone and increased the cytotoxicity of chemotherapy when added (FIGS. 16,17,24,25).

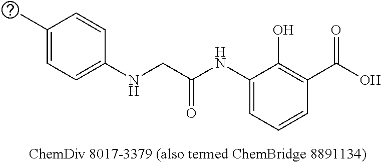

| | |
|---|---|
| MWt | 304 |
| MF | C₁₅H₁₃FN₂O₄ |

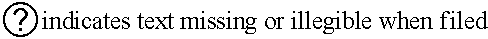

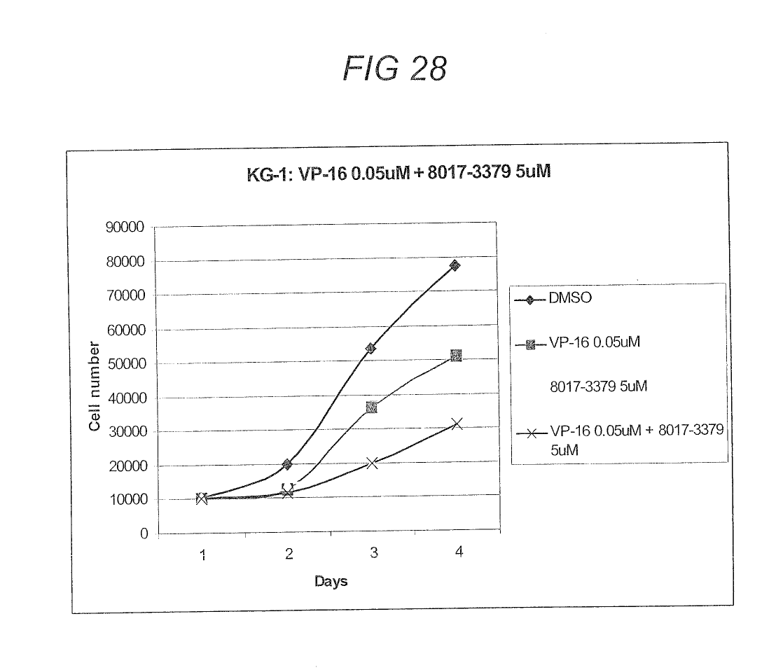

This compound was cytotxoic for cancer cells alone (FIGS. 28,29).

FIG. 16 is a bar graph showing effectiveness of various drugs and combinations of chemical compositions that are Metnase and Intnase in decreasing pancreatic cancer cell growth. The figure shows the results of experiments which were conducted as follows.

### Effects on Pancreatic Cancer

The human Pancreatic cancer cell line BXPC3 was plated (4000 cells per plate with a ˜10% plating efficiency) in 10 cm dishes, using 10 ml of RPMI media (always with 10% FBS) per plate, and placed in a 37®F incubator (5% CO2) for 24 hrs. After 24 hrs, each plate was treated with one or a combination of the following agents: DMSO (0.2% in solution), 0.3 μg/ml of gemcitabine (dissolved in 1×PBS), and/or 2 μM of each Metnase/Intnase inhibitor (dissolved in DMSO). When the DMSO, gemcitabine, and/or inhibitors were added, the plates were swirled several times to assure mixing. After the agents were added to the RPMI, the plates were put back into the incubators for 18 hrs. Following 18 hrs treatment, the media was removed and the cells were washed twice with 1×PBS. Ten ml of fresh RPMI with 10% FBS was added and the cells were placed back into the incubator for 14 days. After the incubation period that will allow surviving cells to form colonies, the media was discarded and surviving colonies were stained with methylene blue. Colonies, a reflection of a single surviving cell that expanded, were counted to measure survival. All experiments were run in triplicate. Experiments were normalized to either DMSO (black asterices) or gemcitabine (red asterices). These data show that D0045-0007, 5483-0023, D058-0127, R092-0025, elvitegravir, and raltegravir have activity on their own against the pancreatic cancer cells, and also potentiate gemcitabine, the best known drug for pancreatic cancer.

FIG. 17 is a bar graph showing effectiveness of various drugs and combinations of chemical compositions in inhibiting pancreatic cancer cell growth.

### Effects on Human Colon Cancer Cell

The human colon cancer cell line SW48 was plated (1000 cells per plate with a ˜20% plating efficiency) in 10 cm dishes, using 10 ml of DMEM media (always with 10% FBS) per plate, and placed in a 37® F incubator (5% CO2) for 24 hrs. After 24 hr, each plate was treated with one or a combination of the following agents: DMSO (0.2% in solution), 3 μM 5-fluorouracil (5FU, dissolved in 1×PBS), and/or 2 μM of each Metnase/intnase inhibitor (dissolved in DMSO). When the DMSO, 5FU, and/or inhibitors were added, the plates were swirled several times to assure mixing. After agents were added to the DMEM, the plates were put back into the incubators for 18 hr. Following 18 hr treatment, the media was removed and the cells were washed twice with 1×PBS. Ten milliliters of fresh DMEM was added and the cells were placed back into the incubator for 7-14 days. After the incubation period that will allow surviving cells to grow and form colonies, media was discarded and surviving colonies were stained with methylene blue. Colonies, a reflection of a single surviving cell that expanded, were counted to measure survival. All experiments were run in triplicate. Experiments were normalized to either DMSO (black asterices) or 5FU (red asterices). These data show that 5483-0023, D058-0127, R092-0025, D0045-0007, and raltegravir have activity on their own against the colon cancer cells, and in some cases (5483-0023) also potentiate 5FU, the best known drug for colon cancer.

### Effects on Leukemia

The human myeloid cell line KG-1 was grown in RPMI1640 with 10% FBS. The small cell lung cancer cell line CRL5898 was grown in HITES medium (Dulbecco's medium: Ham's F12, 50:50 mix, Insulin 0.005 mg/ml Transferrin 0.01 mg/ml Sodium selenite 30 nM Hydrocortisone 10 nM beta-estradiol 10 nM HEPES 10 mM L-glutamine 2 mM (in addition to that in the base medium)). Both cell lines were seeded at 10,000 cells/ml in the appropriate medium in 6 well plates. The Chem Div compounds were resuspended as 5 mM stock solutions in DMSO. The Chem Div compounds were added alone at 2 uM and 5 uM or in combination with VP-16 (also called etoposide) at 0.05 uM. DMSO was added to the control wells. Cells were plated and treated in triplicate and counted daily for 4 days.

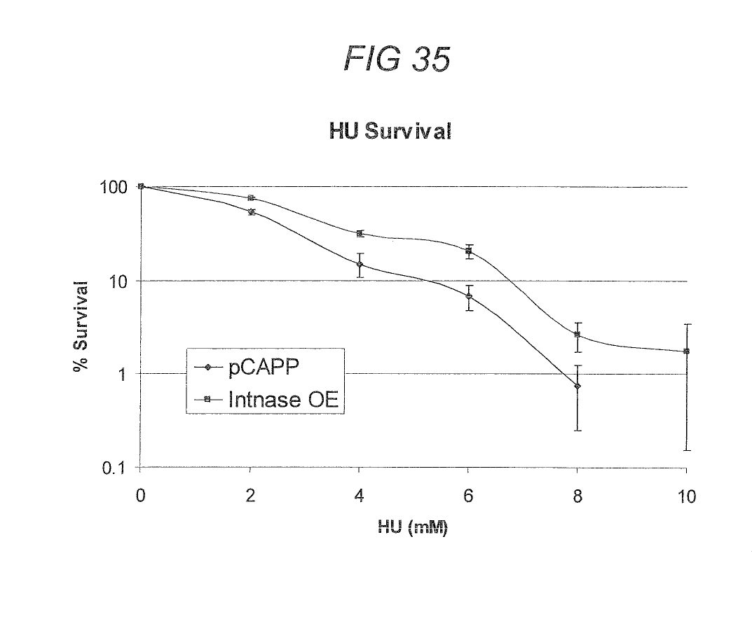

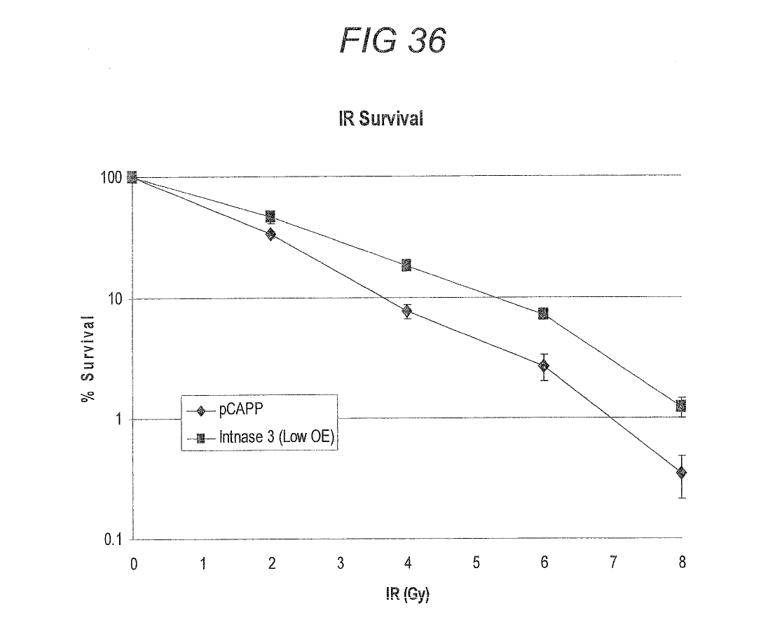

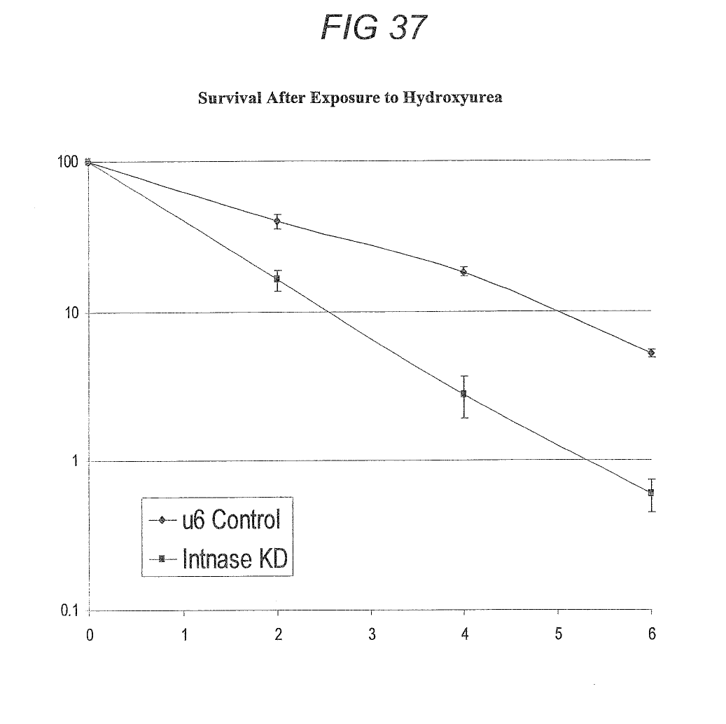

FIGS. 35-37 evidence that Intnase is at least partially responsible for survival rates of cancer cells treated with cancer chemotherapy and/or radiation and that inhibitors of Intnase represent exceptional anti-cancer agents. FIG. 35 shows a colony formation assay of cells that over-express Intnase (here Intnase OE) versus control cells (here pCAPP) performed in the presence of the cancer drug hydroxyurea (here HU), which prevents DNA replication. Cells over-expressing Intnase have an increased survival rate. FIG. 36 shows a colony formation assay indicating that cells that over-express Intnase (here Intnase 3) have an increased survival after exposure to radiation (here IR with dose in Gray, Gy) compared to control cells (here pCAPP). FIG. 37 evidences that Intnase repression using siRNA (here Intnase KID) decreases survival to exposure with hydroxyurea compared to control cells (here U6 control). These three figures are further evidence that Intnase is an appropriate target to increase the effectiveness of cancer treatment.

FIGS. 18-32 show the effect of the various compounds above as depicted against leukemia cell line KG-1 growth curve.

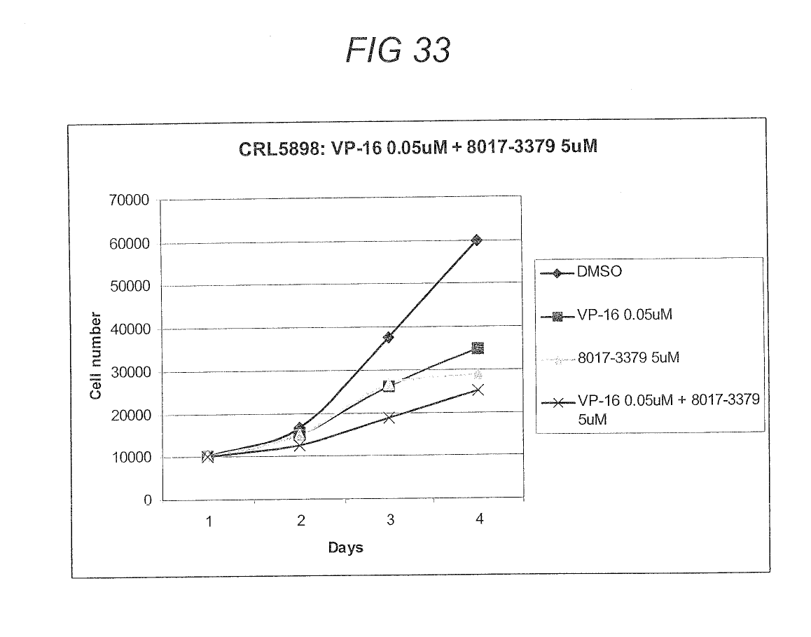

FIG. 33 shows the effect of Raltegravir optionally in the presence of VP-16 on small cell lung cancer cells.

As presented in FIG. 18, compound K828-0009, depicted above, slowed the growth of the leukemia cell line KG-1 more than low dose VP-16 alone and potentiated the activity of VP-16 against this leukemia cell line.

As presented in FIG. 19, compound K511-0010 slowed the growth of the leukemia cell line KG-1 to a greater degree than low dose VP-16 alone. There was some potentiation of the anti-proliferative effect of low dose VP-16 only after longer treatment.

As presented in FIG. 20, a lower dose of compound 5076-4170 slowed the growth of the leukemia cell line KG-1 and caused cell death with prolonged treatment. There was some potentiation on the anti-proliferative effect of low dose VP-16.

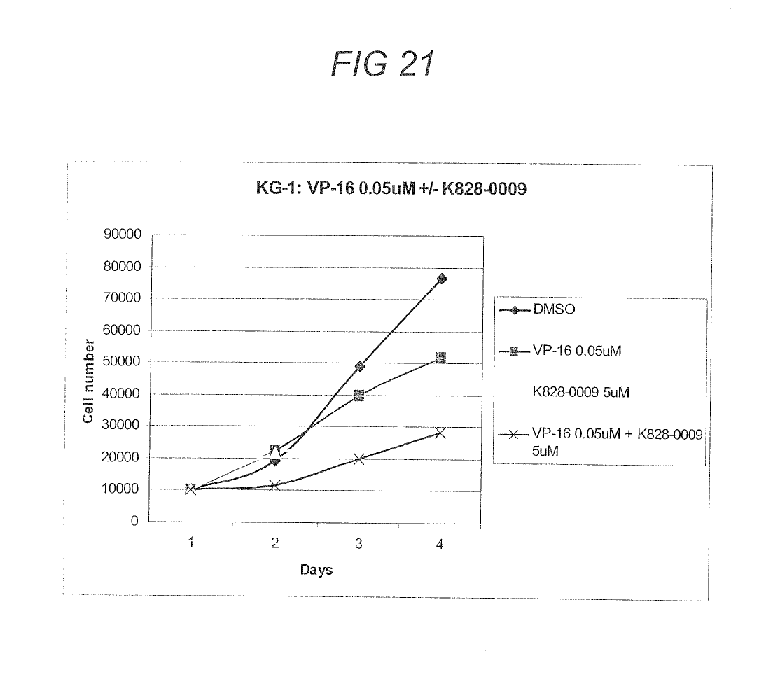

As presented in FIG. 21, a higher dose of the compound 5076-4170 slowed the growth of the leukemia cell line KG-1 and caused cell death with prolonged treatment. There was some potentiation on the anti-proliferative effect of low dose VP-16.

As presented in FIG. 22, a high dose of compound 5082-2438 decreased KG-1 leukemia cell growth, similar to low dose VP-16 alone. It potentiated the anti-proliferative effect of low dose VP-16 and prolonged treatment resulted in cell death.

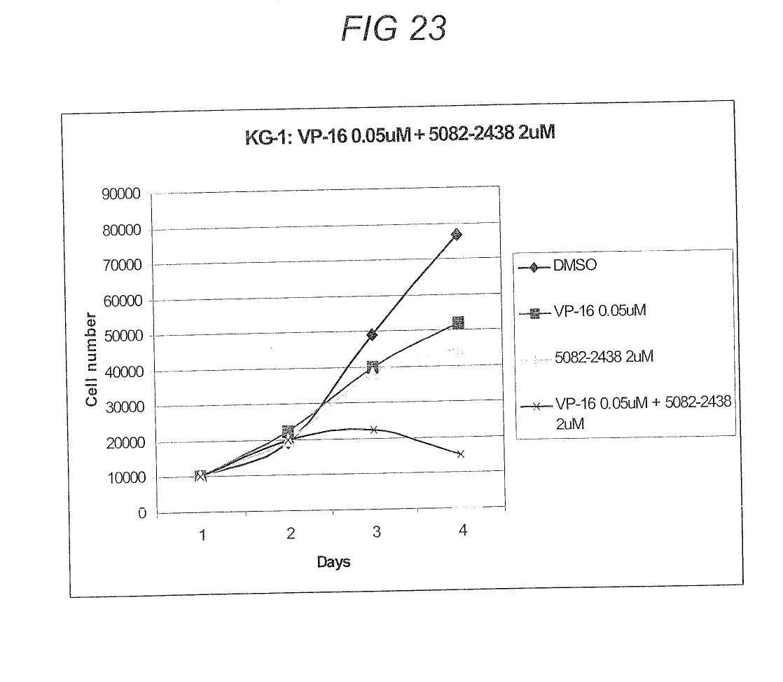

As presented in FIG. 23, a lower dose of compound 5082-2438 decreased KG-1 leukemia cell growth, similar to low dose VP-16 alone. It potentiated the effect of low dose VP-16 and prolonged treatment resulted in cell death.

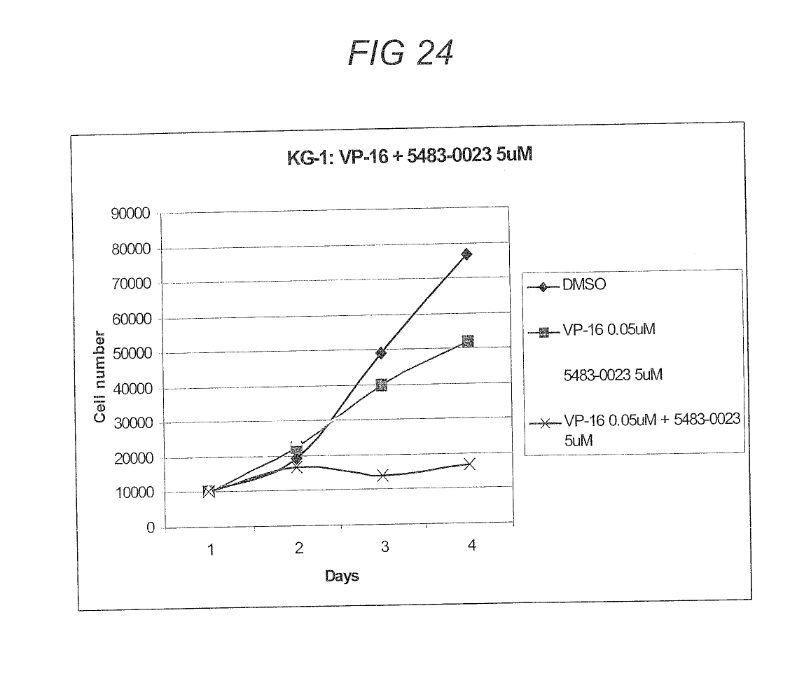

As presented in FIG. 24, a high dose of compound 5483-0023 decreased KG-1 leukemia cell growth especially with longer treatment. As indicated, the compound potentiated the anti-proliferative effect of low dose VP-16.

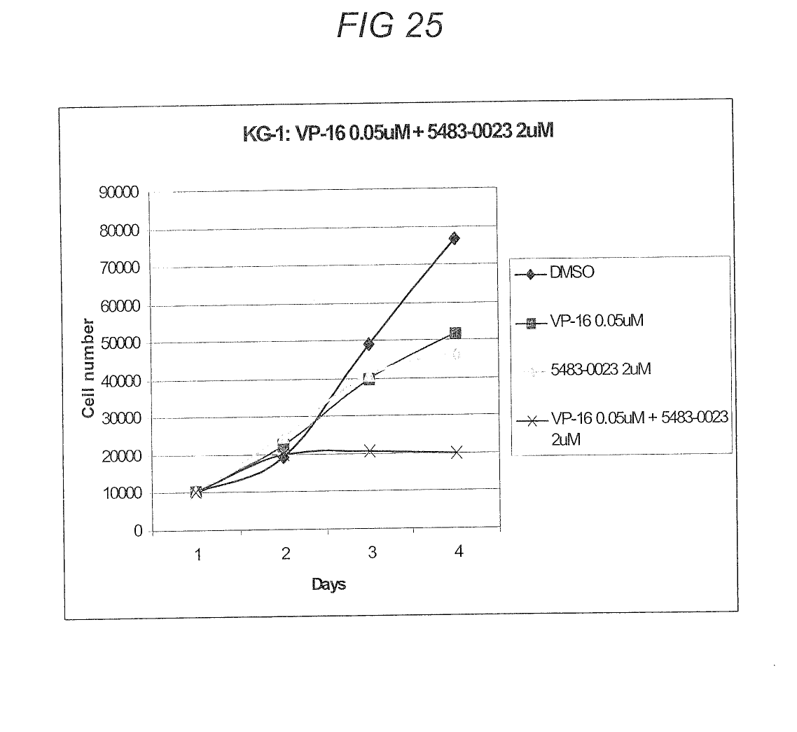

As presented in FIG. 25, a lower dose of compound 5483-0023 decreased KG-1 leukemia cell growth, similar to low dose VP-16. It potentiated the anti-proliferative effect of low dose VP-16.

As presented in FIG. 26, a high dose of compound G396-1171 decreasedKG-1 leukemia cell growth, similar to low dose VP-16 alone. It potentiated the anti-proliferative effect of low dose VP-16.

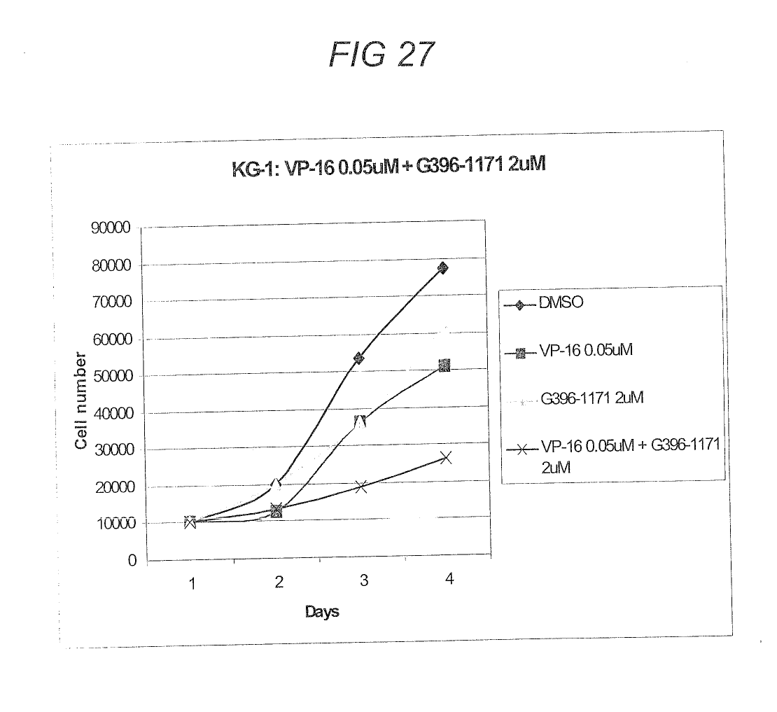

As presented in FIG. 27, a lower dose of compound G396-1171 decreased KG-1 leukemia cell growth, similar to low dose VP-16 alone. It also potentiated the anti-proliferative effect of low dose VP-16.

As presented in FIG. 28, A high dose of compound 8017-3379 inhibited KG-1 leukemia cell growth even more than low dose VP-16 alone. There was no potentiation on the effect of low dose VP-16.

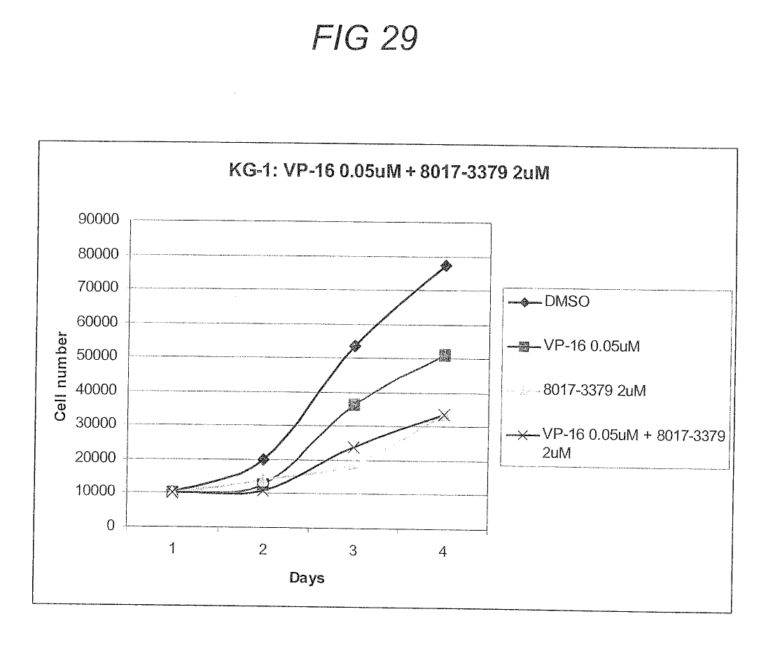

As presented in FIG. 29, a lower dose of compound 8017-3379 inhibited KG-1 leukemia cell growth even more than low dose VP-16 alone. There was no potentiation on the effect of low dose VP-16.

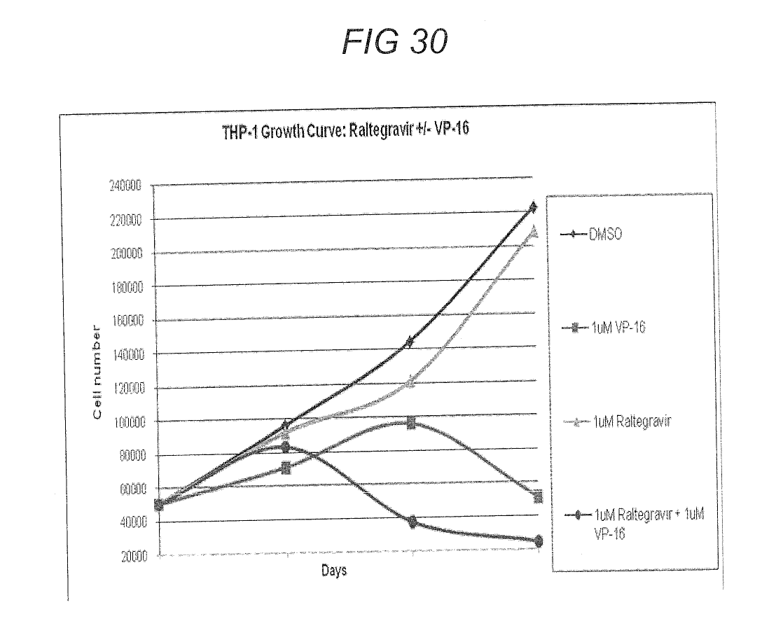

As presented in FIG. 30, a low dose of the HIV integrase inhibitor Raltegravir had little effect on leukemia cell growth. However, raltegravir potentiated the anti-proliferative effect of low dose VP-16.

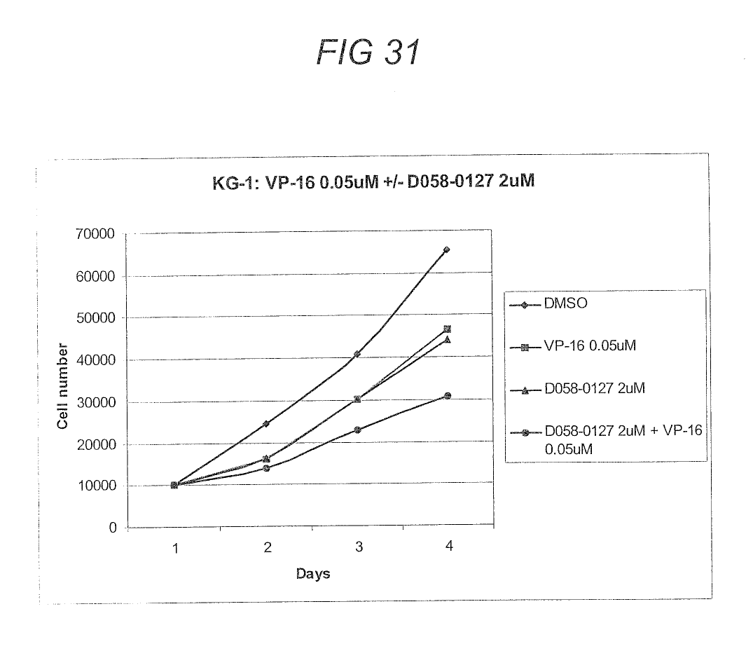

As presented in FIG. 31, a low dose of the integrase inhibitor D058-017 decreased THP-1 leukemia cell growth compared to control. This compound also potentiated the anti-proliferative effect of low dose VP-16.

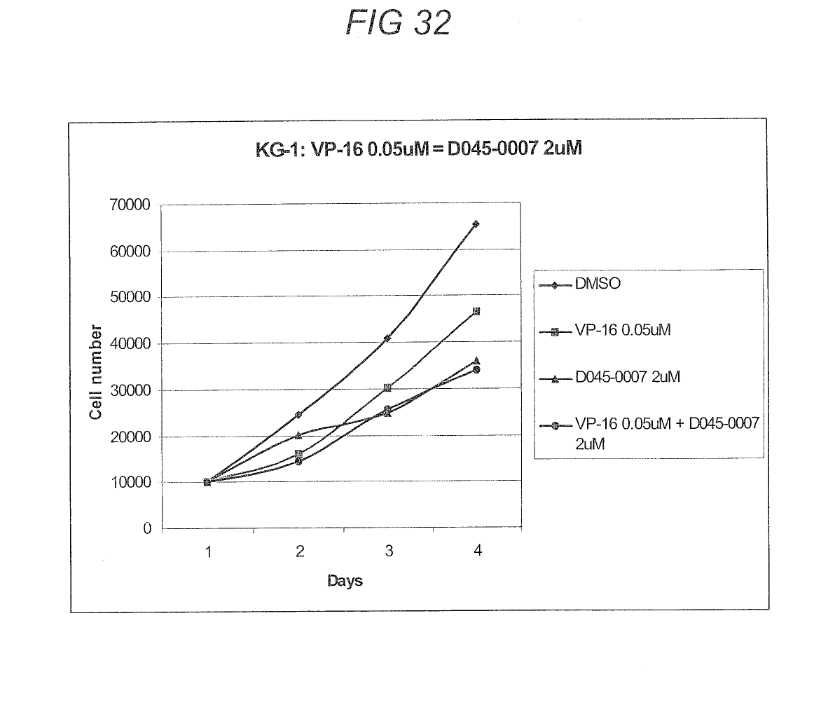

As presented in FIG. 32, a low dose of the integrase inhibitor D045-0007 decreased KG-1 leukemia cell growth compared to control. This compound also potentiated the anti-proliferative effect of low dose VP-16.

As presented in FIG. 33, compound 8017-3379 inhibited cell growth of the small cell lung cancer cell line CRL5898, the same as low dose VP-16, but may have had a greater effect with longer treatment. There was some potentiation of the anti-proliferative effect of low dose VP-16.

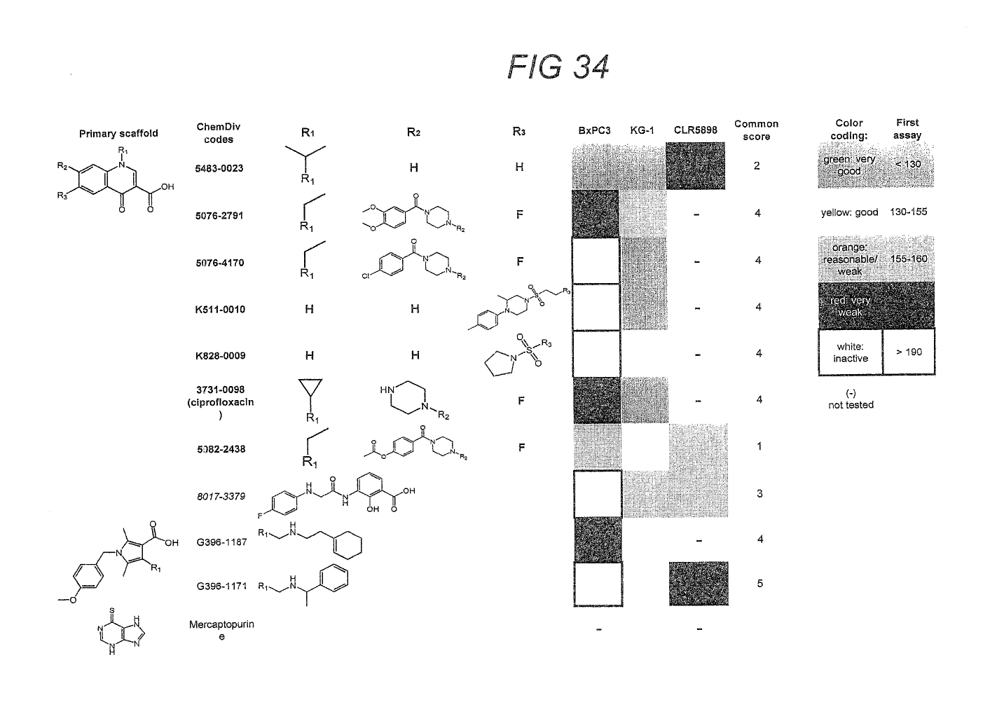

A partial summary of the activities of the compounds on selected cancer cells growth is shown below and in FIG. 34.

| Compound | BxPC3 | KG-1 | CLR5898 |
|---|---|---|---|
| Number | Pancreatic | Leukemia | Small Cell Lung Cancer |
| 5483-0023 | Active | Potency in uM. Cooperativity with VP-16. | Potency in uM. No or very |
| | (potency in | | weak cooperativity with VP-16. |
| | uM) | | |
| 5076-2791 | weak | Potency in uM. Weak cooperativity. | |
| | | High potency, probably below 1 uM. | |
| 5076-4170 | inactive | Reduce concentration of the compound | |
| | | to see if it has any cooperativity with VP- | |
| | | 16 | |
| K511-0010 | inactive | High potency, probably below 1 uM. | |
| | | Reduce concentration of the compound | |
| | | to see if it has any cooperativity with VP- | |
| | | 16 | |
| K828-0009 | inactive | Potency in uM. Cooperativity with VP-16 | |
| | | High potency, probably below 1 uM. | |
| 3731-0098 | weak | Reduce concentration of the compound | |
| | | to see if it has any cooperativity with VP- | |
| | | 16 | |
| G396-1187 | weak | Potency in uM. Cooperativity with VP-16 | |
| 5082-2438 | weak | Potency in uM. Cooperativity with VP-16 | Potency in uM. No or very |
| | | | weak cooperativity with VP-16. |
| G396-1171 | inactive | Potency in uM. Cooperativity with VP-16 | Potency in uM. No |
| | | | cooperativity with VP-16 |
| 8017-3379 | inactive | Potency in uM. No or very weak | Potency in uM. No or very |
| | | cooperativity with VP-16. | weak cooperativity with VP-16. |

The results show that the compounds of FIG. 34 evidenced activity against one or more of the cancer cell lines the drugs were tested in. Note that compounds 8017-03379 and mercaptopurine are final compounds and not substituents.

Separately, the primary scaffold and its derivatives (FIG. 34) were computer modeled to dock in the active sites of the Transposase domains of Metnase, and the Transposase domain of Intnase. The class of compounds that dock to the Transposase domain active site, shown by the primary scaffold structure, includes the known HIV integrase inhibitor Elvitegravir. We also disclose that another related HIV integrase inhibitor Raltegravir, closely related to this class of compounds, docks to the active site of the Transposase domains of Metnase and Intnase, and also potentiates the anti-proliferative activity of VP-16, gemcitabine and 5-fluorouracil. These compounds are not only active agents in potentiating DNA damaging cancer chemotherapy such as those listed above, but on their own they show anti-proliferative activity against cancer cells. Consequently, the compounds disclosed herein are evidenced to be Transposase domain inhibitors which can be utilized as drugs to treat cancer, either alone or in concert with other known chemotherapeutic agents or with radiation therapy.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. Any inconsistency between the material incorporated by reference and the material set for in the specification as originally filed shall be resolved in favor of the specification as originally filed. The foregoing detailed description and examples have been given for clarity of understanding only. No unnecessary limitations are to be understood therefrom. The invention is not limited to the exact details shown and described, for variations obvious to one skilled in the art will be included within the invention defined by the following claims.

All headings are for the convenience of the reader and should not be used to limit the meaning of the text that follows the heading, unless so specified.

1. A method of treating cancer in a patient comprising administering to said patient an effective amount of at least one Metnase and/or Intnase inhibitor.

2. The method according to claim 1 wherein said patient is also administered an effective amount of second agent useful in treating cancer.

3. The method according to claim 1 wherein said patient is also treated with radiation therapy.

4. The method according to claim 1 wherein said Metnase and/or inhibitor is a compound according to the chemical structure: 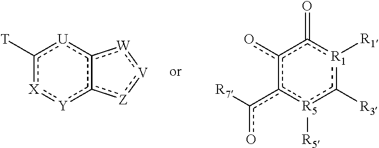
Where U is 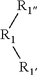
a group;
V is a 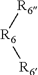
group or a 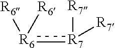
group;
W is a 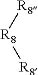
group;
X is a 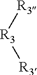
group;
Y is a 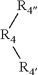
group;
Z is a 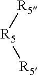
group;
R₁, R₃, R₄, R₅, R₆, R₇ and R₈ are each independently, C, C═O, N, O, S, S═O, or 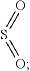
R_{1′}, R_{3′}, R_{4′}, R_{5′}, R_{6′}, R_{7′}, and R_{8′} are each independently absent or a C₁-C₆ optionally substituted linear, branched or cyclic alkyl group, a halogen (F, Cl, Br, I), cyano, nitro, nitroso, azido, hydroxyl, thiol, (CH₂)ₙ-aryl which is optionally substituted, (CH₂)ₙ-heterocycle which is optionally substituted, C₁-C₆ optionally substituted alkoxy, (CH₂), —C₁-C₆ optionally substituted ester, (CH₂)ₙ—C₁-C₆ optionally substituted thioester, C₁-C₆ optionally substituted ether, C₁-C₆ optionally substituted thioether, (CH₂)ₙ—C₁-C₆ optionally substituted acyl (keto) group, (CH₂)ₙ—C₁-C₆ optionally substituted diketo group, (CH₂)ₙ—C₁-C₆ optionally substituted thioacyl (thioketo) group, (CH₂)ₙ—C₁-C₆ optionally substituted carboxylic acid, (CH₂)ₙ—C₁-C₆ optionally substituted thioic acid, (CH₂)ₙ—C₁-C₆ optionally substituted sulfone, (CH₂)ₙ—C₁-C₆ optionally substituted sulfonate, (CH₂)ₙ—C₁-C₆ optionally substituted sulfate, (CH₂)ₙ—C₁-C₆ optionally substituted sulfoxide, (CH₂)ₙ—C₁-C₆ optionally substituted sulfonamide, (CH₂), —C₁-C₆ optionally substituted sulfoximide, (CH₂)ₙ—NR¹R² wherein R₁ and R₂ are each independently H, or a C₁-C₃ alkyl group optionally substituted with at least one hydroxyl group; C₁-C₆ optionally substituted diamine, a (CH₂)ₙ-triazene (N—N═N) group which is optionally substituted with one or two C₁-C₆ alkyl groups which are themselves optionally substituted with at least one hydroxyl group, an optionally substituted C₁-C₆ guanidino group, an optionally substituted (CH₂)ₙ-amide group, an optionally substituted (CH₂)ₙ-thioamide group, an optionally substituted (CH₂)ₙ-amidine group, a (CH₂)ₙ-diazo group, an optionally substituted (CH₂)ₙ-diazonium group, an optionally substituted carbamodithioic group, an optionally substituted (CH₂)ₙ-urea group, an optionally substituted (CH₂)ₙ-thiourea group, an optionally substituted (CH₂)ₙ-hydrazine group, an optionally substituted (CH₂)ₙ-hydrazide, an optionally substituted (CH₂)ₙ-isocyanate, an optionally substituted (CH₂)ₙ-thiocyanate, an optionally substituted (CH₂)ₙ-carbonate, an optionally substituted (CH₂)ₙ-carbamate, an optionally substituted (CH₂)ₙ-phosphonate or an optionally substituted (C₁₋₁₂)ₙ-phosphate, or when R₁, R₃, R₄, R₅, R₆, R₇ or R₈ is a carbon atom, R_{1′}, together with R_{1″}, R_{3′} together with R_{3″}, R_{4′} together with R_{4″}, R_{5′} together with R_{5″}, R_{6′} together with R_{6″}, R_{7′} together with R_{7″}, and R_{8′} together with R_{8″} may optionally form an optionally substituted double bond with said carbon atom, or one or more of R_{1′}, R_{3′} and R_{4′} may optionally form a 5 to 20-membered carbocyclic or heterocyclic ring or fused ring system with T (preferably R^{3′} forms a 5 to 7-membered carbocyclic or heterocyclic ring with T);
R_{1″}, R_{3″},R_{4″}, R_{5″}, R_{6″}, R_{7″}, and R_{8″} are each independently absent, a C₁-C₁₀ optionally substituted hydrocarbon group, preferably an optionally substituted C₁-C₆ linear, branched or cyclic alkyl group, a halogen (F, Cl, Br, I), cyano, nitro, nitroso, azido, hydroxyl, thiol, (CH₂)ₙ-heterocycle which is optionally substituted, C₁-C₆ optionally substituted alkoxy, (CH₂)ₙ—C₁-C₆ optionally substituted ester, (CH₂)ₙ—C₁-C₆ optionally substituted thioester, C₁-C₆ optionally substituted ether, C₁-C₆ optionally substituted thioether, (CH₂)ₙ—C₁-C₆ optionally substituted acyl (keto) group, (CH₂)ₙ—C₁-C₆ optionally substituted diketo group, (CH₂)ₙ—C₁-C₆ optionally substituted thioacyl (thioketo) group, (CH₂)ₙ—C₁-C₆ optionally substituted carboxylic acid, (CH₂)ₙ—NR¹R² wherein R₁ and R₂ are each independently H, or a C₁-C₃ alkyl group optionally substituted with at least one hydroxyl group; C₁-C₆ diamine which is optionally substituted with one or two C₁-C₆ alkyl groups which are themselves optionally substituted with at least one hydroxyl group, a (CH₂)ₙ-triazene (N—N═N) group which is optionally substituted with one or two C₁-C₆ alkyl groups which are themselves optionally substituted with at least one hydroxyl group, an optionally substituted C₁-C₆ guanidino group wherein the terminal amine is optionally substituted one or two C₁-C₆ alkyl groups which are themselves optionally substituted with at least one hydroxyl group, an optionally substituted (CH₂)ₙ-amide group, an optionally substituted (CH₂)ₙ-thioamide group, an optionally substituted (CH₂)ₙ-amidine group, an optionally substituted (CH₂)ₙ-urea group, an optionally substituted (CH₂)ₙ-thiourea group, an optionally substituted
(CH₂)ₙ-hydrazine group, an optionally substituted (CH₂)ₙ-hydrazide, an optionally substituted (CH₂)ₙ-carbonate, an optionally substituted (CH₂)ₙ-carbamate, an optionally substituted (CH₂)ₙ-phosphonate, an optionally substituted (CH₂)ₙ-phosphate, or
when R₁, R₃, R₄, R₅, R₆, R₇ or R₈ is a carbon atom, R_{1″}, together with R_{1′}, R_{3″} together with R_{3′}, R_{4″} together with R_{4′}, R_{5″} together with R_{5′}, R_{6″} together with R_{6′}, R_{7″} together with R_{7′}, and R_{8″} together with R_{8′} may optionally form a double bond with said carbon atom which is optionally substituted;
T is a O—R_{9′} group, a C(O)OR_{10′} group, a O—C(O)R_{10′} group or forms a 5 to 20-membererd carbocyclic or heterocyclic ring or fused ring system with one or more of R_{1′}, R_{3′} and R_{4′} (preferably T forms a 5 to 7-membered carbocyclic or heterocyclic ring with R^{3′});
R_{9′}, is a C₁-C₆ hydrocarbon, preferably a linear branched or cyclic alkyl group which is optionally substituted, a (CH₂)ⱼ—C₁-C₆ ether or thioether group which is optionally substituted, a (CH₂)ⱼ—C₁-C₆ acyl group which is optionally substituted, a (CH₂)ⱼ—NR¹R² group wherein R₁ and R₂ are each independently H, or a C₁-C₃ alkyl group optionally substituted with at least one hydroxyl group, an optionally substituted (CH₂)ₙ-amide group, an optionally substituted (CH₂)ₙ-thioamide group, an optionally substituted (CH₂)ₙ-aryl group or an optionally substituted (CH₂)ₙ-heterocyclic group;
R_{10′} is a C₁-C₆ hydrocarbon, preferably a linear branched or cyclic alkyl group which is optionally substituted, a (CH₂)ⱼ—C₁-C₆ ether or thioether group which is optionally substituted, a (CH₂)ⱼ—C₁-C₆ acyl group which is optionally substituted, a (CH₂)ⱼ—NR¹R² group wherein R₁ and R₂ are each independently H, or a C₁-C₃ alkyl group optionally substituted with at least one hydroxyl group, an optionally substituted (CH₂)ₙ-amide group, an optionally substituted (CH₂)ₙ-thioamide group, an optionally substituted (CH₂)ₙ-aryl group or an optionally substituted (CH₂)ₙ-heterocyclic group;
j is 1, 2, 3, 4, 5 or 6, preferably 1, 2 or 3;
n is 0, 1, 2, 3, 4, 5, or 6, preferably 0, 1, 2, or 3;
Or a pharmaceutically acceptable salt, solvate or polymorph thereof.

5. The method according to claim 1 wherein said metnase and/or intnase inhibitor is a compound according to the chemical structure: 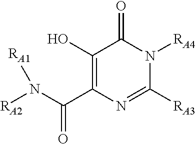
Where R_{A1} is H or a C₁-C₆ alkyl group which is optionally substituted with at least one hydroxyl or halogen group;
R_{A2} is (1) H;
(2) C₁-C₆ alkyl which is optionally substituted with one or more substituents each of which is independently halogen, —OH, O—C₁-C₆ alkyl, —O—C₁-C₆ haloalkyl, —NO₂, —N(R^{a}R^{b}), —C(═O)R^{a}, —CO₂R^{a}, —SR^{a}, —S(═O)R^{a}, —SO₂R^{a}, or
—N(R^{a})CO₂R^{b},
(3) C₁-C₆ alkyl which is optionally substituted with one or more substituents each of which is independently halogen, —OH, or O—C₁₋₄ alkyl, and which is substituted with 1 or 2 substituents each of which is independently:
(i) C₃-C₈ cycloalkyl,
(ii) aryl,
(iii) a fused bicyclic carbocycle consisting of a benzene ring fused to a C₅-C₇ cycloalkyl,
(iv) a 5- or 6-membered saturated heterocyclic ring containing from 1 to 4 heteroatoms independently selected from N, O and S,
(v) a 5- or 6-membered heteroaromatic ring containing from 1 to 4 heteroatoms independently selected from N, O and S, or
(vi) a 9- or 10-membered fused bicyclic heterocycle containing from 1 to 4 heteroatoms independently selected from N, O and S, wherein at least one of the rings is aromatic,
(4) C₂-C₅ alkynyl optionally substituted with aryl,
(5) C₃-C₈ cycloalkyl optionally substituted with aryl,
(6) aryl,
(7) a fused bicyclic carbocycle consisting of a benzene ring fused to a C₅-C₇ cycloalkyl,
(8) a 5- or 6-membered saturated heterocyclic ring containing from 1 to 4 heteroatoms independently selected from N, O and S,
(9) a 5- or 6-membered heteroaromatic ring containing from 1 to 4 heteroatoms independently selected from N, O and S, or
(10) a 9- or 10-membered fused bicyclic heterocycle containing from 1 to 4 heteroatoms independently selected from N, O and S, wherein at least one of the rings is aromatic;
wherein each aryl in (2)(ii) or the aryl (3), (4) or (5) or each fused carbocycle in (2)(iii) or the fused carbocycle in (6) is optionally substituted with one or more substituents each of which is independently halogen, —OH, —C₁-C₆ alkyl, —C₁-C₆ alkyl-ORa,
—C₁-C₆ haloalkyl, —O—C₁-C₆ alkyl, —O—C1-6 haloalkyl, —CN, —NO₂, —N(RaRb), —C₁-C₆ alkyl-N(R^{a}R^{b}), —C(═O)N(R^{a}R^{b}), —C(═O)R^{a}, —CO₂R^{a},
—C₁-C₆alkyl-CO₂R^{a}, —OCO₂R^{a}, —SR^{a}, —S(═O)R^{a}, —SO₂R^{a}, —N(R^{a})SO₂R^{b},
—SO₂N(R^{a}R^{b}), —N(R^{a})C(═O)R^{b}, —N(R^{a})CO₂R^{b}, —C₁-C₆ alkyl-N(R^{a})CO₂R^{b}, aryl,
—C₁-C₆ alkyl-aryl, —O-aryl, or —C₀-C₆ alkyl-het wherein het is a 5- or 6-membered heteroaromatic ring containing from 1 to 4 heteroatoms independently selected from N, O and S, and het is optionally fused with a benzene ring, and is further optionally substituted with one or more substituents each of which is independently —C₁-C₆ alkyl, —C₁-C₆ haloalkyl, —O—C₁-C₆ alkyl, —O—C₁-C₆ haloalkyl, oxo (═O), or —CO₂Rₐ;
each saturated heterocyclic ring in (2)(iv) or the saturated heterocyclic ring in (7) is optionally substituted with one or more substituents each of which is independently halogen, —C₁-C₆ alkyl, —C₁-C₆ haloalkyl, —O—C₁-C₆ alkyl, —O—C₁-C₆ haloalkyl, oxo, aryl, or a 5- or 6-membered heteroaromatic ring containing from 1 to 4 heteroatoms independently selected from N, O and S; and
each heteroaromatic ring in (2)(v) or the heteroaromatic ring in (8) or each fused bicyclic heterocycle in (2)(vi) or the fused bicyclic heterocycle in (9) is optionally substituted with one or more substituents each of which is independently halogen, —C₁-C₆ alkyl,
—C₁-C₆ haloalkyl, —O—C₁-C₆ alkyl, —O—C₁-C₆ haloalkyl, oxo, aryl, or —C₁-C₆ alkyl-aryl; or alternatively R^{a} and R^{b} together with the N to which both are attached form a C₃-C₇ azacycloalkyl which is optionally substituted with one or more substituents each of which is independently —C₁-C₆ alkyl or oxo;
each R^{a}, R^{b}, R^{c}, and R^{d} is independently —H or —C₁-C₆ alkyl which is optionally substituted with at least one hydroxyl group;
R^{k} is a carbocycle or heterocycle, wherein the carbocycle or heterocycle is optionally substituted with one or more substituents each of which is independently
(1) halogen,
(2) —OH,
(3) —CN,
(4) —C₁-C₆ alkyl, which is optionally substituted with one or more substituents each of which is independently halogen, —OH, —CN, —O—C₁-C₆ alkyl, —O—C₁-C₆ haloalkyl, —C(═O)R^{a}, —CO₂R^{a}, —SR^{a}, —S(═O)R^{a}, —N(R^{a}R^{b}),
—C(═O)—(CH₂)₀₋₂N(R^{a}R^{b}), N(R^{a})—C(═O)—(CH₂)₀₋₂N(R^{b}R^{c}), —SO₂Ra,
—N(R^{a})SO₂R^{b}, —SO₂N(R^{a}R^{b}), or N(R^{a})—C(R^{b})═O,
(5) —O—C₁-C₆ alkyl, which is optionally substituted with one or more substituents each of which is independently halogen, —OH, —CN, —O—C₁-C₆ alkyl, —O—C₁-C₆haloalkyl, —C(═O)R^{a}, —CO₂R^{a}, —SR^{a}, —S(═O)R^{a}, —N(R^{a}R^{b}),
—C(═O)—(CH₂)₀₋₂N(R^{a}R^{b}), N(R^{a})—C(═O)—(CH₂)₀₋₂N(R^{b}R^{c}), —SO₂R^{a},
—N(R^{a})SO₂R^{b}, —SO₂N(R^{a}R^{b}), or —N(R^{a})—C(R^{b})═O,
(6) —NO₂,
(7) oxo,
(8) —C(═O)R^{a},
(9) —CO₂R^{a},
(10) —SR^{a},
(11) —S(═O)R^{a},
(12) —N(R^{a}R^{b}),
(13) —C(═O)N(R^{a}R^{b}),
(14) —C(═O)—C₁-C₆ alkyl-N(R^{a}R^{b}),
(15) —N(R^{a})C(═O)R^{b},
(16) —SO₂R^{a},
(17) —SO₂N(R^{a}R^{b}),
(18) —N(R^{a})SO₂R^{b},
(19) —R^{m},
(20) —C₁-C₆ alkyl-R^{m}, wherein the alkyl is optionally substituted with one or more substituents each of which is independently halogen, —OH, —CN, —C₁-C₆ haloalkyl, —O—C₁-C₆ alkyl, —O—C₁-C₆ haloalkyl, —C(═O)R^{a}, —CO₂R^{a}, —SR^{a}, —S(═O)R^{a}, N(R^{a}R^{b}), —N(R^{a})CO₂R^{b}, —SO₂Ra, —N(Ra)SO₂Rb, —SO₂N(RaRb), or —N(Ra)—C(Rb)═O,
(21) —C₀-C₆ alkyl-N(R^{a})—C₀-C₆ alkyl-R^{m},
(22) —C₀-C₆ alkyl-O—C₀-C₆ alkyl-R^{m},
(23) —C₀-C₆ alkyl-S—C₀-C₆ alkyl-R^{m},
(24) —C₀-C₆ alkyl-C(═O)—C₁-C₆ alkyl-R^{m},
(25) —C(═O)—O—C₀-C₆ alkyl-R^{m},
(26) —C(═O)N(R^{a})—C₀-C₆ alkyl-R^{m},
(27) —N(R^{a})C(═O)—R^{m},
(28) —N(R^{a})C(═O)—C₁-C₆ alkyl-R^{m}, wherein the alkyl is optionally substituted with one or more substituents each of which is independently halogen, —OH, —CN, —C₁-C₆ haloalkyl, —O—C₁-C₆ alkyl, —O—C₁-C₆ haloalkyl, —C(═O)R^{a}, —CO₂R^{a}, —SR^{a},
—S(O)R^{a}, —N(R^{a}R^{b}), —N(R^{a})CO₂R^{b}, —SO₂R^{a}, —N(R^{a})SO₂Rb, —SO₂N(RaR^{b}), or
—N(R^{a})—C(R^{b})═O,
(29) —N(R^{a})—C(O)—N(R^{b})—C₀-C₆ alkyl-R^{m},
(30) —N(R^{a})—C(═O)—O—C₀-C₆ alkyl-R^{m},
(31) —N(R^{a})—C(═O)N(R^{b})—SO₂—C₀-C₆ alkyl-R^{m},
(32) —C(═O)—C(═O)—N(R^{a}R^{b}),
(33) —C(═O)—C₁-C₆ alkyl-SO₂R^{a}, or
(34) —C(═O)—C(═O)R^{m};
wherein the carbocycle in R^{k} is (i) a C₃ to C₈ monocyclic, saturated or unsaturated ring, (ii) a C₇ to C₁₂ bicyclic ring system, or (iii) a C₁₁ to C₁₆ tricyclic ring system, wherein each ring in (ii) or (iii) is independent of or fused to the other ring or rings and each ring is saturated or unsaturated;
the heterocycle in R^{k} is (i) a 4- to 8-membered, saturated or unsaturated monocyclic ring, (ii) a 7- to 12-membered bicyclic ring system, or (iii) an 11 to 16-membered tricyclic ring system; wherein each ring in (ii) or (iii) is independent of or fused to the other ring or rings and each ring is saturated or unsaturated; the monocyclic ring, bicyclic ring system, or tricyclic ring system contains from 1 to 6 heteroatoms selected from N, O and S and a balance of carbon atoms; and wherein any one or more of the nitrogen and sulfur heteroatoms is optionally be oxidized, and any one or more of the nitrogen heteroatoms is optionally quaternized;
each R^{m} is independently a C₃-C₈ cycloalkyl; aryl; a 5- to 8-membered monocyclic heterocycle which is saturated or unsaturated and contains from 1 to 4 heteroatoms independently selected from N, O and S; or a 9- to 10-membered bicyclic heterocycle which is saturated or unsaturated and contains from 1 to 4 heteroatoms independently selected from N, O and S; wherein any one or more of the nitrogen and sulfur heteroatoms in the heterocycle or bicyclic heterocycle is optionally oxidized and any one or more of the nitrogen heteroatoms is optionally quaternized; and wherein
the cycloalkyl or the aryl of R^{m} is optionally substituted with one or more substituents each of which is independently halogen, —C₁-C₆ alkyl optionally substituted with —O—C₁-C₄ alkyl, —C₁-C₆ haloalkyl, —O—C₁-C₆ alkyl, —O—C₁-C₆ haloalkyl, —N(R^{a}R^{b}), aryl, or —C₁-C₆ alkyl-aryl; and
the monocyclic or bicyclic heterocycle defined in Rm is optionally substituted with one or more substituents each of which is independently halogen, —C₁-C₆ alkyl, —C₁-C₆ haloalkyl, —O—C₁-C₆ alkyl, —O—C₁-C₆ haloalkyl, oxo, aryl, —C₁-C₆ alkyl-aryl,
—C(═O)-aryl, —CO₂-aryl, —CO₂—C₁-C₆ alkyl-aryl, a 5- or 6-membered saturated heterocyclic ring containing from 1 to 4 heteroatoms independently selected from N, O and S, or a 5- or 6-membered heteroaromatic ring containing from 1 to 4 heteroatoms independently selected from N, O and S; and
each n is independently an integer equal to zero, 1 or 2;
RA3 is
(1) —H,
(2) —C₁-C₆ alkyl, which is optionally substituted with one or more substituents each of which is independently halogen, —OH, —CN, —O—C₁-C₆ alkyl, —O—C₁-C₆ haloalkyl, —C(═O)R^{a}, —CO₂R^{a}, —SR^{a}, —S(═O)R^{a}, —N(R^{a}R^{b}), —C(═O)—C₀-C₆ alkyl-N(R^{a}R^{b}), N(R^{a})—C(═O)—C₀-C₆ alkyl-N(R^{b}R^{c}), —SO₂R^{a}, —N(R^{a})SO₂R^{b},
—SO₂N(R^{a}R^{b}),
—N(R^{a})—C(═O)R^{b}, or —N(R^{a})C(═O)C(═O)N(R^{a}R^{b}),
(3) —R^{k},
(4) —C₁-C₆ alkyl-R^{k}, wherein:
(i) the alkyl is optionally substituted with one or more substituents each of which is independently halogen, —OH, —CN, —O—C₁-C₆ alkyl, —O—C₁-C₆ haloalkyl, —N(R^{a}R^{b}), —N(R^{a})CO₂R^{b}, —N(R^{a})C(═O)—C₀-C₆ alkyl-N(R^{b}R^{c}), or —N(R^{a})—C₂-C₆ alkyl-OH with the proviso that the —OH is not attached to the carbon alpha to N(R^{a}); and
(ii) the alkyl is optionally mono-substituted with —R^{S}, —C₁-C₆ alkyl-R^{S}, —N(R^{a})—C(═O)—C₀-C₆ alkyl-R^{S}, —N(R^{a})—C₀-C₆ alkyl-R^{s}, —O—C₀-C₆ alkyl-R^{S}, or —N(R^{a})—C(═O)—C₀-C₆ alkyl-R^{S};
wherein R^{S} is
(a) aryl which is optionally substituted with one or more substituents each of which is independently halogen, —OH, —C₁-C₆ alkyl, —C₁-C₆ alkyl-OR^{a}, —C₁-C₆ haloalkyl,
—O—C₁-C₆ alkyl, —O—C₁-C₆ haloalkyl, methylenedioxy attached to two adjacent carbon atoms, or aryl;
(b) a 4- to 8-membered saturated heterocyclic ring containing from 1 to 4 heteroatoms independently selected from N, O and S; wherein the saturated heterocyclic ring is optionally substituted with one or more substituents each of which is independently halogen, —C₁-C₆C₁₋₆ alkyl, —C₁-C₆ alkyl-OR^{a}, —C₁-C₆ haloalkyl, —O—C₁-C₆ alkyl,
—O—C₁-C₆ haloalkyl, —C(═O)R^{a}, —CO₂R^{a}, —C(═O)—C₀-C₆ alkyl-N(R^{a}R^{b}),
—SO₂R^{a}, oxo, aryl, or —C₁-C₆ alkyl-aryl; or
(c) a 5- to 7-membered heteroaromatic ring containing from 1 to 4 heteroatoms independently selected from N, O and S; wherein the heteroaromatic ring is optionally substituted with one or more substituents each of which is independently halogen,
—C₁-C₆ alkyl, —C₁-C₆ alkyl-OR^{a}, —C₁-C₆ haloalkyl, —O—C₁-C₆ alkyl, —O—C₁-C₆ haloalkyl, oxo, or aryl;
(5) —C₀-C₆ alkyl-O—C₀-C₆ alkyl-R^{k},
(6) —C₀-C₆ alkyl-S(O)ₙ—C₁-C₆ alkyl-R^{k},
(7) —O—C₁-C₆ alkyl-OR^{k},
(8) —O—C₁₋₆ alkyl-O—C₁₋₆ alkyl-R^{k},
(9) —O—C₁₋₆ alkyl-S(O)ₙR^{k},
(10) —C₀₋₆ alkyl-N(R^{a})—R^{k},
(11) —C₀₋₆ alkyl-N(R^{a})—C₁₋₆ alkyl-R^{k},
(12) —C₀₋₆ alkyl-N(R^{a})—C₁₋₆ alkyl-OR^{k},
(13) —C₀₋₆ alkyl-C(═O)—R^{k},
(14) —C₀₋₆ alkyl-C(═O)N(R^{a})—C₀₋₆ alkyl-R^{k},
(15) —C₀₋₆ alkyl-N(R^{a})C(═O)—C₀₋₆ alkyl-R^{k},
(16) —C₀₋₆ alkyl-N(R^{a})C(═O)—O—C₀₋₆ alkyl-R^{k}, or
(17) —C₀₋₆ alkyl-N(R^{a})C(═O)C(═O)R^{k};
RA4 is
—C₁₋₆ alkyl which is optionally substituted with one or more substituents each of which is independently
(1) halogen,
(2) —OH,
(3) —CN,
(4) —O—C₁₋₆ alkyl,
(5) —O—C₁₋₆ haloalkyl,
(6) —C(═O)R^{a},
(7) —CO₂R^{a},
(8) —SR^{a},
(9) —S(═O)R^{a},
(10) —N(R^{a}R^{b}),
(11) —C(═O)N(R^{a}R^{b}),
(12) —N(R^{a})—C(═O)—C₁₋₆ alkyl-N(R^{b}R^{c}),
(13) —SO₂R^{a},
(14) —N(R^{a})SO₂R^{b},
(15) —SO₂N(R^{a}R^{b}),
(16) —N(R^{a})—C(R^{b})═O,
(17) —C₃₋₈ cycloalkyl,
(18) aryl,
wherein the aryl is optionally substituted with one or more substituents each of which is independently halogen, —C₁₋₆ alkyl, —C₁₋₆ haloalkyl, —O—C₁₋₆ alkyl, —O—C₁₋₆ haloalkyl, —C₀₋₆ alkyl-N(R^{a}R^{b}), or —C₁₋₆ alkyl substituted with a 5- or 6-membered saturated heterocyclic ring containing from 1 to 4 heteroatoms independently selected from N, O and S; wherein the saturated heterocyclic ring is optionally substituted with from 1 to 3 substituents each of which is independently —C₁₋₆ alkyl, oxo, or a 5- or 6-membered heteroaromatic ring containing from 1 to 4 heteroatoms independently selected from N, O and S; or
(19) a 5- to 8-membered monocyclic heterocycle which is saturated or unsaturated and contains from 1 to 4 heteroatoms independently selected from N, O and S; wherein the heterocycle is optionally substituted with one or more substituents each of which is independently —C₁₋₆ alkyl, —O—C₁₋₆ alkyl, oxo, phenyl, or naphthyl;
with the proviso that none of the following substituents is attached to the carbon atom in the —C₁₋₆alkyl group that is attached to the ring nitrogen: halogen, —OH, —O—C₁₋₆ alkyl, —O—C₁₋₆ haloalkyl, —SR^{a}, —S(═O)R^{a}, —N(R^{a}R^{b}), or —N(R^{a})—C(R^{b})═O;
Or a pharmaceutically acceptable salt, solvate or polymorph thereof.

6. The method according to claim 1 wherein said metnase and/or intnase inhibitor is
4-oxo-1,4-dihyroquinoline-3-carboxylic acid;
4-oxo-4H-thio chromene-3-carboxylic acid;
2-oxo-1,2-dihydroquinoline-3-carboxylic acid;
1-hydroxy-2-naphthoic acid;
4-oxo-1,4,5,6,7,8-hexahydroquinoline-3-carboxylic acid;
3-hydroxy-2-naphthoic acid;
1,4-dioxo-1,4-dihydronaphthalene-2-carboxylic acid;
4-oxo-1,4-dihydro-1,8-naphthyridine-3-carboxylic acid;
7-oxo-4,7-dihydrothiazolo[5,4-b]pyridine-6-carboxylic acid;
4-acetyl-3-hydroxypyridin-2(1H)-one;
5-hydroxy-6-oxo-1,6-dihydropyrimidine-4-carboxamide;
8-hydroxy-3,4-dihydro-2,6-naphthyridine-1,7(2H,6H)-dione;
1-hydroxy-4-methoxy-5,5,8,8-tetramethyl-5,6,7,8-tetrahydronaphthalene-2-carboxylic acid;
4,8-dioxo-4,8-dihydrothiopyrano[3,2-b]thiopyran-3,7-dicarboxylic acid;
4-(3,4-dimethoxyphenyl)-1-hydroxy-5,6,7-trimethoxynaphthalene-2,3-dicarboxylic acid;
7-(3-aminopyrrolidin-1-yl)-1-cyclopropyl-4-oxo-1,4-dihydro-1,8-naphthyndine-3-carboxylic acid;
1-ethyl-6-fluoro-7-(2-((methylamino)methyl)pyrimidin-4-yl)-4-oxo-1,4-dihydroquinoline-3-carboxylic acid;
7-chloro-4-hydroxy-2,3.dihydro-1H-indene-5-carboxylic acid;
6-(3-chloro-4-fluorobenzyl)-4-hydroxy-2-isopropyl-N,N-dimethyl-3,5-dioxo-2,3,5,6,7,8-hexahydro-2,6-naphthyridine-1-carboxamide;
4-(5-(4-fluorobenzyl)furan-2-carbonyl)-3-hydroxy-1-methylpyridine-2(1H)-one;
7-fluoro-5-oxo-1-(phenylthio)-8-(piperazin-1-yl)-2,5-dihydro-1H-thiazolo[3,2-1]quinoline-4-carboxylic acid;
4-(7-chloro-2,4-dioxo-1,2-dihydroquinazolin-3(4H)-ylsulfonyl)-1-hydroxy-2-naphthoic acid;
7-(1-amino-5-azaspiro[2,5]octan-5-yl)-1-cyclopropyl-6-fluoro-4-oxo-1,4-dihydro-1,8-naphthyridine-3-carboxylic acid;
2-nitro-4-oxo-7a,8,9,10,11,11a-hexahydro-4H-pyrido[3,2,1-jk]carbazole-5-carboxylic acid;
7,7′-(2,2′-(methylazanediyl)bis(ethane-2,1-diyl)bis(oxy)bis(1-ethyl-4-oxo-1,4-dihydro-1,8-naphthyridine-3-carboxylic acid);
4-[3-(3-carboxy-4-hydroynaphthalen-1-yl)-3-oxopropanoyl]-1-hydroxynaphthalene-2-carboxylic acid;
N-(2-(4-(4-fluorobenzylcarbamoyl)-5-hydroxy-1-methyl-6-oxo-1,6-dihydropyrimidin-2-yl)propan-2-yl)-5-methyl-1,3,4-oxadiazole-2-carb oxamide (Raltegravir);
(S)-6-(3-chloro-2-fluorobenzyl)-1-(1-hydroxy-3-methylbutan-2-yl)-7-methoxy-4-oxo-1,4-dihydroquinoline-3-carboxylic acid (Elvitegravir);
phenyl 1-hydroxy-2-naphthoate;
thiazol-4-ylmethyl 1,4-dihydroxy-2-naphthoate;
methyl 6-hydroxy-2,2-dimethyl-2H-benzo[h]chromene-5-carboxylate (Rubimaillin);
2-(2,5-dimethyl-1-((tetrahydrofuran-2-yl)methyl)-1H-pyrrol-3-yl)-2-oxoethyl 1,4-dihydroxy-2-naphthoate;
(S)-9,10-dihydroxy-7-methoxy-3-methyl-3,4-dihydro-1H-benzo[g]isochromene-1-one (Semivioxanthin);
9,10-dihydroxy-5,7-dimethoxy-3-methyl-1H-benzo[g]isochromene-1-one (Paepalantine);
Elsamitrucin;
Rubioncolin B, or a pharmaceutically acceptable salt thereof or a mixture thereof.

7. The method according to claim 6 wherein said metnase and/or intnase inhibitor is mercaptopurine, 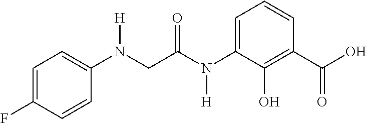
or a compound according to the chemical structure: 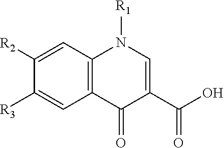
Where R₁ is H, a C₁-C₃ alkyl group which is linear, branch-chained or cyclic alkyl group,
Or a group according to the chemical structure: 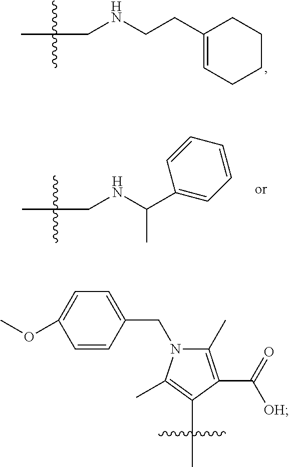
R₂ is H, a halogen (preferably F) or a group according to the chemical structure 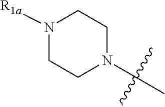
where R₁ₐ is H, a C₁-C₃ linear, branch-chained or cyclic alkyl group, or a C(O)—R₂ₐ group, where R₂ₐ is a phenyl group which is optionally substituted with one or two C₂-C₄ oxycarbonyl or carboxylic acid groups, or one, two or three halogen or C₁-C₃ alkoxy groups; and
R₃ is H, a halogen (preferably F) or a (CH₂)ₙ—S(O)₂—R₃ₐ group where n is 0 to 3; and
R₃ₐ is an optionally substituted 5- or 6-membered heterocyclic group having one, two or three O, S or N heteroatoms, or
a pharmaceutically acceptable salt, solvate or polymorph thereof.

8. The method according to claim 1 wherein said cancer is selected from the group consisting of carcinomas, including squamous-cell carcinomas, adenocarcinomas, hepatocellular carcinomas, and renal cell carcinomas, bladder, bowel, breast, cervix, uterine, testicular, colon, esophagus, head, kidney, liver, lung, including small cell lung cancer, neck, ovary, pancreas, prostate, thyroid, esophageal, and stomach cancer, leukemia, benign and malignant lymphoma, Burkitt's lymphoma and Non-Hodgkin's lymphoma, benign and malignant melanomas, non-melanoma skin cancer, cutaneous malignancy, myeloproliferative diseases, Ewing's sarcoma, hemangiosarcoma, Kaposi's sarcoma, liposarcoma, myosarcomas, peripheral neuroepithelioma, and synovial sarcoma; gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; germ-line tumors, including bowel cancer, breast cancer, prostate cancer, cervical cancer, uterine cancer, lung cancer, ovarian cancer, testicular cancer, thyroid cancer, esophageal cancer, pancreatic cancer, stomach cancer, liver cancer, colon cancer, and melanoma; mixed types of neoplasia, particularly carcinosarcoma and Hodgkin's disease; and tumors of mixed origin, such as Wilms' tumor and teratocarcinomas prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma.

9. The method according to claim 1 wherein said cancer is leukemia, small cell lung cancer or pancreatic cancer.

10. The method according to claim 8 wherein said metnase and/or intnase inhibitor is raltegravir, elvitegravir, semivioxanthin, paepalantine, elsamitrucin,
rubioncolin B, pharmaceutically acceptable salts and mixtures thereof.

11. The method according to claim 10 wherein said metnase and/or intnase inhibitor is coadministered to said patient with an effective amount of a second agent useful as an anticancer agent.

12. The method according to claim 11 wherein said anticancer agent is selected from the group consisting of antimetabolites, inhibitors of topoisomerase I and II, alkylating agents, microtubule inhibitors and mixtures thereof.

13. The method according to claim 11 wherein said second agent is selected from the group consisting of adriamyucin, aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate; and mixtures thereof.

14. The method according to claim 11 wherein said second agent is etoposide (VP-16), gemcitabine, 5-fluoruracil (5FU) or a mixture thereof.

15. A method of potentiating the effects of an anticancer agent in a therapeutic treatment of cancer in a patient, comprising coadministering to said patient along with said anticancer agent an effective amount of a metnase and/or intnase inhibitor.

16. The method according to claim 15 wherein said anticancer agent is selected from the group consisting of antimetabolites, inhibitors of topoisomerase I and II, alkylating agents, microtubule inhibitors and mixtures thereof.

17. The method according to claim 15 wherein said anticancer agent is selected from the group consisting of adriamyucin, aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate; and mixtures thereof.

18. The method according to claim 15 wherein said anticancer agent is selected from the group consisting of etoposide (VP-16), gemcitabine, 5-fluorouracil and mixtures thereof and said metnase and/or intnase inhibitor is selected from the group consisting of raltegravir, elvitegravir, semivioxanthin, paepalantine, elsamitrucin, rubioncolin B, pharmaceutically acceptable salts and mixtures thereof.

19. The method according to claim 1 wherein said patient is other than an HIV positive, AIDS or ARC patient.

20. The method according to claim 1 wherein said patient is a patient to whom integrase inhibitors are contraindicated.

21. A pharmaceutical composition comprising a combination of an effective amount of a metnase and/or intnase inhibitor in combination with a second compound which is useful as an anticancer agent, in further combination with a pharmaceutically acceptable carrier, additive or excipient.

22. A pharmaceutical composition comprising a combination of an effective amount of a metnase and/or intnase inhibitor selected from the group consisting of raltegravir, elvitegravir, semivioxanthin, paepalantine, elsamitrucin, rubioncolin B, pharmaceutically acceptable salts and mixtures thereof, in combination with an anticancer agent selected from the group consisting of gemcitabine, etoposide (VP-16), 5-fluorouracil and mixtures thereof.

23-42. (canceled)

43. The method according to claim 15 wherein said patient is other than an HIV positive, AIDS or ARC patient.

44. The method according to claim 15 wherein said patient is a patient to whom integrase inhibitors are contraindicated.


SEQUENCE LISTING (1 sequence)

SEQ ID NO: 1 - protein, 522 aa, Homo sapiens
MVRSGKNGDLHLKQIAYYKRTGEYHSTTLPSERSGIRRAAKKFVFKEKKLFYVGKDRKQNRLVIVSEEEKKKVLRECHENDSGAHHGISRTLTLVESNYYWTSVTNDVKQWVYACQHCQVAKNTVIVAPKQHLLKVENPWSLVTVDLMGPFHTSNRSHVYAIIMTDLFTKWIVILPLCDVSASEVSKAIINIFFLYGPPQKIIMDQRDEFIQQINIELYRLFGIKQIVISHTSGTVNPTESTPNTIKAFLSKHCADHPNNWDDHLSAVSFAFNVTHLEPTKNTPYFQMFSRNPYMPETSDSLHEVDGDNTSMFAKILDAIKEADKIMENKTTSLGQMENNNLDELNKSKIIVKKKPKQLNPFHLKVGHEVLRQRKNWWKDGRFQSEWVGPCVIDYITESGCAVLRDNTGVRLKRPIKMSHLKPYIRESSEQESLYLLQGSVVADHDYIGLPEIPIGAYQANILVEDATIGIVDNELLTSSKDRELLEYRNTKISPLIDDHSSLEKQTFSLLDSSNQVLEYLS